SEQUENCE LISTING (76 sequences)

SEQ ID NO: 1 - DNA, 24 bp, synthetic construct
  source 1..24: mol_type = other DNA; note = Primer CROP-seq sgRNA amplicon forward
atcttgtggaaaggacgaaacacc

SEQ ID NO: 2 - DNA, 23 bp, synthetic construct
  source 1..23: mol_type = other DNA; note = Primer CROP-seq sgRNA amplicon rev
tgtctcaagatctagttacgcca

SEQ ID NO: 3 - DNA, 25 bp, synthetic construct
  source 1..25: mol_type = other DNA; note = Primer oRT_CROPseq
gactagccttattttaacttgctat

SEQ ID NO: 4 - DNA, 77 bp, synthetic construct
  modified_base 1: mod_base = OTHER; note = phosphorylation
  source 1..77: mol_type = other DNA; note = Primer oPD_CROPseq
gttttagagctagaaatagcaagctcctgttcgacacctacccacctcatcccactcttcaaaaggacgaaacaccg

SEQ ID NO: 5 - DNA, 35 bp, synthetic construct
  source 1..35: mol_type = other DNA; note = Primer oSBS_CROPseq
cacctcatcccactcttcaaaaggacgaaacaccg

SEQ ID NO: 6 - DNA, 28 bp, synthetic construct
  source 1..28: mol_type = other DNA; note = multiplexed amplicon sequencing barcodes FWD1_1
gagaaatcttgtggaaaggacgaaacac

SEQ ID NO: 7 - DNA, 28 bp, synthetic construct
  source 1..28: mol_type = other DNA; note = multiplexed amplicon sequencing barcodes FWD1_2
caagaatcttgtggaaaggacgaaacac

SEQ ID NO: 8 - DNA, 28 bp, synthetic construct
  source 1..28: mol_type = other DNA; note = multiplexed amplicon sequencing barcodes FWD1_3
gaacaatcttgtggaaaggacgaaacac

SEQ ID NO: 9 - DNA, 28 bp, synthetic construct
  source 1..28: mol_type = other DNA; note = multiplexed amplicon sequencing barcodes FWD1_4
ccataatcttgtggaaaggacgaaacac

SEQ ID NO: 10 - DNA, 28 bp, synthetic construct
  source 1..28: mol_type = other DNA; note = multiplexed amplicon sequencing barcodes FWD1_5
gttagatcttgtggaaaggacgaaacac

SEQ ID NO: 11 - DNA, 28 bp, synthetic construct
  source 1..28: mol_type = other DNA; note = multiplexed amplicon sequencing barcodes FWD1_6
actcgatcttgtggaaaggacgaaacac

SEQ ID NO: 12 - DNA, 28 bp, synthetic construct
  source 1..28: mol_type = other DNA; note = multiplexed amplicon sequencing barcodes FWD1_7
tgttgatcttgtggaaaggacgaaacac

SEQ ID NO: 13 - DNA, 28 bp, synthetic construct
  source 1..28: mol_type = other DNA; note = multiplexed amplicon sequencing barcodes FWD1_8
aggtcatcttgtggaaaggacgaaacac

SEQ ID NO: 14 - DNA, 28 bp, synthetic construct
  source 1..28: mol_type = other DNA; note = multiplexed amplicon sequencing barcodes FWD1_9
aggaaatcttgtggaaaggacgaaacac

SEQ ID NO: 15 - DNA, 28 bp, synthetic construct
  source 1..28: mol_type = other DNA; note = multiplexed amplicon sequencing barcodes FWD1_10
acagaatcttgtggaaaggacgaaacac

SEQ ID NO: 16 - DNA, 28 bp, synthetic construct
  source 1..28: mol_type = other DNA; note = multiplexed amplicon sequencing barcodes FWD1_11
agacaatcttgtggaaaggacgaaacac

SEQ ID NO: 17 - DNA, 28 bp, synthetic construct
  source 1..28: mol_type = other DNA; note = multiplexed amplicon sequencing barcodes FWD1_12
tggtaatcttgtggaaaggacgaaacac

SEQ ID NO: 18 - DNA, 28 bp, synthetic construct
  source 1..28: mol_type = other DNA; note = multiplexed amplicon sequencing barcodes FWD2_1
ctaggatcttgtggaaaggacgaaacac

SEQ ID NO: 19 - DNA, 28 bp, synthetic construct
  source 1..28: mol_type = other DNA; note = multiplexed amplicon sequencing barcodes FWD2_2
cgatgatcttgtggaaaggacgaaacac

SEQ ID NO: 20 - DNA, 28 bp, synthetic construct
  source 1..28: mol_type = other DNA; note = multiplexed amplicon sequencing barcodes FWD2_3
ttcgcatcttgtggaaaggacgaaacac

SEQ ID NO: 21 - DNA, 28 bp, synthetic construct
  source 1..28: mol_type = other DNA; note = multiplexed amplicon sequencing barcodes FWD2_4
ggttcatcttgtggaaaggacgaaacac

SEQ ID NO: 22 - DNA, 28 bp, synthetic construct
  source 1..28: mol_type = other DNA; note = multiplexed amplicon sequencing barcodes FWD2_5
ccgaaatcttgtggaaaggacgaaacac

SEQ ID NO: 23 - DNA, 28 bp, synthetic construct
  source 1..28: mol_type = other DNA; note = multiplexed amplicon sequencing barcodes FWD2_6
tcggaatcttgtggaaaggacgaaacac

SEQ ID NO: 24 - DNA, 28 bp, synthetic construct
  source 1..28: mol_type = other DNA; note = multiplexed amplicon sequencing barcodes FWD2_7
aagcaatcttgtggaaaggacgaaacac

SEQ ID NO: 25 - DNA, 28 bp, synthetic construct
  source 1..28: mol_type = other DNA; note = multiplexed amplicon sequencing barcodes FWD2_8
tcctaatcttgtggaaaggacgaaacac

SEQ ID NO: 26 - DNA, 28 bp, synthetic construct
  source 1..28: mol_type = other DNA; note = multiplexed amplicon sequencing barcodes FWD2_9
aatggatcttgtggaaaggacgaaacac

SEQ ID NO: 27 - DNA, 28 bp, synthetic construct
  source 1..28: mol_type = other DNA; note = multiplexed amplicon sequencing barcodes FWD2_10
gcatgatcttgtggaaaggacgaaacac

SEQ ID NO: 28 - DNA, 28 bp, synthetic construct
  source 1..28: mol_type = other DNA; note = multiplexed amplicon sequencing barcodes FWD2_11
tatgcatcttgtggaaaggacgaaacac

SEQ ID NO: 29 - DNA, 28 bp, synthetic construct
  source 1..28: mol_type = other DNA; note = multiplexed amplicon sequencing barcodes FWD2_12
cctgtatcttgtggaaaggacgaaacac

SEQ ID NO: 30 - DNA, 28 bp, synthetic construct
  source 1..28: mol_type = other DNA; note = multiplexed amplicon sequencing barcodes FWD3_1
gtggaatcttgtggaaaggacgaaacac

SEQ ID NO: 31 - DNA, 28 bp, synthetic construct
  source 1..28: mol_type = other DNA; note = multiplexed amplicon sequencing barcodes FWD3_2
ctgcaatcttgtggaaaggacgaaacac

SEQ ID NO: 32 - DNA, 28 bp, synthetic construct
  source 1..28: mol_type = other DNA; note = multiplexed amplicon sequencing barcodes FWD3_3
accaaatcttgtggaaaggacgaaacac

SEQ ID NO: 33 - DNA, 28 bp, synthetic construct
  source 1..28: mol_type = other DNA; note = multiplexed amplicon sequencing barcodes FWD3_4
tgcgaatcttgtggaaaggacgaaacac

SEQ ID NO: 34 - DNA, 28 bp, synthetic construct
  source 1..28: mol_type = other DNA; note = multiplexed amplicon sequencing barcodes FWD3_5
tgtcaatcttgtggaaaggacgaaacac

SEQ ID NO: 35 - DNA, 28 bp, synthetic construct
  source 1..28: mol_type = other DNA; note = multiplexed amplicon sequencing barcodes FWD3_6
tagagatcttgtggaaaggacgaaacac

SEQ ID NO: 36 - DNA, 28 bp, synthetic construct
  source 1..28: mol_type = other DNA; note = multiplexed amplicon sequencing barcodes FWD3_7
taccgatcttgtggaaaggacgaaacac

SEQ ID NO: 37 - DNA, 28 bp, synthetic construct
  source 1..28: mol_type = other DNA; note = multiplexed amplicon sequencing barcodes FWD3_8
ttgtgatcttgtggaaaggacgaaacac

SEQ ID NO: 38 - DNA, 28 bp, synthetic construct
  source 1..28: mol_type = other DNA; note = multiplexed amplicon sequencing barcodes FWD3_9
acaccatcttgtggaaaggacgaaacac

SEQ ID NO: 39 - DNA, 28 bp, synthetic construct
  source 1..28: mol_type = other DNA; note = multiplexed amplicon sequencing barcodes FWD3_10
accttatcttgtggaaaggacgaaacac

SEQ ID NO: 40 - DNA, 28 bp, synthetic construct
  source 1..28: mol_type = other DNA; note = multiplexed amplicon sequencing barcodes FWD3_11
ctctaatcttgtggaaaggacgaaacac

SEQ ID NO: 41 - DNA, 28 bp, synthetic construct
  source 1..28: mol_type = other DNA; note = multiplexed amplicon sequencing barcodes FWD3_12
tcacgatcttgtggaaaggacgaaacac

SEQ ID NO: 42 - DNA, 28 bp, synthetic construct
  source 1..28: mol_type = other DNA; note = multiplexed amplicon sequencing barcodes REV1_A
ggcaatgtctcaagatctagttacgcca

SEQ ID NO: 43 - DNA, 28 bp, synthetic construct
  source 1..28: mol_type = other DNA; note = multiplexed amplicon sequencing barcodes REV1_B
aacgatgtctcaagatctagttacgcca

SEQ ID NO: 44 - DNA, 28 bp, synthetic construct
  source 1..28: mol_type = other DNA; note = multiplexed amplicon sequencing barcodes REV1_C
gtccatgtctcaagatctagttacgcca

SEQ ID NO: 45 - DNA, 28 bp, synthetic construct
  source 1..28: mol_type = other DNA; note = multiplexed amplicon sequencing barcodes REV1_D
tgaagtgtctcaagatctagttacgcca

SEQ ID NO: 46 - DNA, 28 bp, synthetic construct
  source 1..28: mol_type = other DNA; note = multiplexed amplicon sequencing barcodes REV1_E
gtacgtgtctcaagatctagttacgcca

SEQ ID NO: 47 - DNA, 28 bp, synthetic construct
  source 1..28: mol_type = other DNA; note = multiplexed amplicon sequencing barcodes REV1_F
acgtgtgtctcaagatctagttacgcca

SEQ ID NO: 48 - DNA, 28 bp, synthetic construct
  source 1..28: mol_type = other DNA; note = multiplexed amplicon sequencing barcodes REV1_G
caacctgtctcaagatctagttacgcca

SEQ ID NO: 49 - DNA, 28 bp, synthetic construct
  source 1..28: mol_type = other DNA; note = multiplexed amplicon sequencing barcodes REV1_H
cactttgtctcaagatctagttacgcca

SEQ ID NO: 50 - DNA, 28 bp, synthetic construct
  source 1..28: mol_type = other DNA; note = multiplexed amplicon sequencing barcodes REV2_A
cgtaatgtctcaagatctagttacgcca

SEQ ID NO: 51 - DNA, 28 bp, synthetic construct
  source 1..28: mol_type = other DNA; note = multiplexed amplicon sequencing barcodes REV2_B
ggtgatgtctcaagatctagttacgcca

SEQ ID NO: 52 - DNA, 28 bp, synthetic construct
  source 1..28: mol_type = other DNA; note = multiplexed amplicon sequencing barcodes REV2_C
cctcatgtctcaagatctagttacgcca

SEQ ID NO: 53 - DNA, 28 bp, synthetic construct
  source 1..28: mol_type = other DNA; note = multiplexed amplicon sequencing barcodes REV2_D
atgagtgtctcaagatctagttacgcca

SEQ ID NO: 54 - DNA, 28 bp, synthetic construct
  source 1..28: mol_type = other DNA; note = multiplexed amplicon sequencing barcodes REV2_E
atccgtgtctcaagatctagttacgcca

SEQ ID NO: 55 - DNA, 28 bp, synthetic construct
  source 1..28: mol_type = other DNA; note = multiplexed amplicon sequencing barcodes REV2_F
gactgtgtctcaagatctagttacgcca

SEQ ID NO: 56 - DNA, 28 bp, synthetic construct
  source 1..28: mol_type = other DNA; note = multiplexed amplicon sequencing barcodes REV2_G
tctcctgtctcaagatctagttacgcca

SEQ ID NO: 57 - DNA, 28 bp, synthetic construct
  source 1..28: mol_type = other DNA; note = multiplexed amplicon sequencing barcodes REV2_H
gctaatgtctcaagatctagttacgcca

SEQ ID NO: 58 - DNA, 28 bp, synthetic construct
  source 1..28: mol_type = other DNA; note = multiplexed amplicon sequencing barcodes REV3_A
cttgatgtctcaagatctagttacgcca

SEQ ID NO: 59 - DNA, 28 bp, synthetic construct
  source 1..28: mol_type = other DNA; note = multiplexed amplicon sequencing barcodes REV3_B
ggatatgtctcaagatctagttacgcca

SEQ ID NO: 60 - DNA, 28 bp, synthetic construct
  source 1..28: mol_type = other DNA; note = multiplexed amplicon sequencing barcodes REV3_C
agtagtgtctcaagatctagttacgcca

SEQ ID NO: 61 - DNA, 28 bp, synthetic construct
  source 1..28: mol_type = other DNA; note = multiplexed amplicon sequencing barcodes REV3_D
gatcgtgtctcaagatctagttacgcca

SEQ ID NO: 62 - DNA, 28 bp, synthetic construct
  source 1..28: mol_type = other DNA; note = multiplexed amplicon sequencing barcodes REV3_E
agctgtgtctcaagatctagttacgcca

SEQ ID NO: 63 - DNA, 28 bp, synthetic construct
  source 1..28: mol_type = other DNA; note = multiplexed amplicon sequencing barcodes REV3_F
cttcctgtctcaagatctagttacgcca

SEQ ID NO: 64 - DNA, 28 bp, synthetic construct
  source 1..28: mol_type = other DNA; note = multiplexed amplicon sequencing barcodes REV3_G
attgctgtctcaagatctagttacgcca

SEQ ID NO: 65 - DNA, 28 bp, synthetic construct
  source 1..28: mol_type = other DNA; note = multiplexed amplicon sequencing barcodes REV3_H
gtgcttgtctcaagatctagttacgcca

SEQ ID NO: 66 - DNA, 1116 bp, synthetic construct
  source 1..1116: mol_type = other DNA; note = GFP generic donor plasmid (relevant part)
atgttctttcctgcgttatcccctggagatcgagtgccgcatcaccggctattggtcttactgacatccactttgcctttctctccacagggtggcggtggctcgggcggtggtgggtccggtggcggcggatctggcggtggtggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcggcggtggatccggtggcggcggatctggcggtggaggttcgggtggcggtgggtcgggcaaggtaagtatcaattagaagacgaattcccggtgatgcggcactcgatctcgaattcttacagacaagctgtgaccgtctccgggagctgcatgtgtcagaggttttcaccgtcatcaccgaaacgcgcgagacgaaagggcctcgtgatacgcctatttttataggttaatgtcatgataataatgg

SEQ ID NO: 67 - DNA, 5071 bp, synthetic construct
  source 1..5071: mol_type = other DNA; note = GFP minicircle
acattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactagtgaattcagatctgatcctgcgttatcccctggagatcgagtgccgcatcaccggctattggtcttactgacatccactttgcctttctctccacagggtggcggtggctcgggcggtggtgggtccggtggcggcggatctggcggtggtggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcggcggtggatccggtggcggcggatctggcggtggaggttcgggtggcggtgggtcgggcaaggtaagtatcaattagaagacgaattccatctctagagtcgacccatgggggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgat

SEQ ID NO: 68 - DNA, 5053 bp, synthetic construct
  source 1..5053: mol_type = other DNA; note = mScarlet frame 1 minicircle
acattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactagtgaattcagatctgatcctgcgttatcccctggagatcgagtgccgcatcaccggctattggtcttactgacatccactttgcctttctctccacagggggtggcggtggctcgggcggtggtgggtccggtggcggcggatctggcggtggtggatccgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagggcggcggtggatccggtggcggcggatctggcggtggaggttcgggtggcggtgggtcgggcggaggtaagtatcaattagaagacgaattccatctctagagtcgacccatgggggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgat

SEQ ID NO: 69 - DNA, 196 bp, synthetic construct
  source 1..196: mol_type = other DNA; note = Part of EGFP minicircle - forward strand
gagatcgagtgccgcatcaccggctattggtcttactgacatccactttgcctttctctccacagggtggcggtggctcgggcggtggtgggtccggtggcggcggatctggcggtggtggatccgtgagcaagggcgaggagctgttcaccggggtggtgcccatcctggtcgagctggacggcgacgtaaacgg

SEQ ID NO: 70 - DNA, 196 bp, synthetic construct
  source 1..196: mol_type = other DNA; note = Part of EGFP minicircle - reverse complement
ccgtttacgtcgccgtccagctcgaccaggatgggcaccaccccggtgaacagctcctcgcccttgctcacggatccaccaccgccagatccgccgccaccggacccaccaccgcccgagccaccgccaccctgtggagagaaaggcaaagtggatgtcagtaagaccaatagccggtgatgcggcactcgatctc

SEQ ID NO: 71 - DNA, 143 bp, synthetic construct
  source 1..143: mol_type = other DNA; note = Part of EGFP minicircle forward strand
ggagttcgtgaccgccgccgggatcactctcggcatggacgagctgtacaagggcggcggtggatccggtggcggcggatctggcggtggaggttcgggtggcggtgggtcgggcaaggtaagtatcaattagaagacgaatt

SEQ ID NO: 72 - DNA, 143 bp, synthetic construct
  source 1..143: mol_type = other DNA; note = Part of EGFP minicircle - reverse complement
aattcgtcttctaattgatacttaccttgcccgacccaccgccacccgaacctccaccgccagatccgccgccaccggatccaccgccgcccttgtacagctcgtccatgccgagagtgatcccggcggcggtcacgaactcc

SEQ ID NO: 73 - DNA, 175 bp, synthetic construct
  source 1..175: mol_type = other DNA; note = Part of mScarlet minicircle - forward strand
gagatcgagtgccgcatcaccggctattggtcttactgacatccactttgcctttctctccacagggggtggcggtggctcgggcggtggtgggtccggtggcggcggatctggcggtggtggatccgtgagcaagggcgaggcagtgatcaaggagttcatgcggttcaaggtg

SEQ ID NO: 74 - DNA, 175 bp, synthetic construct
  source 1..175: mol_type = other DNA; note = Part of mScarlet mincircle - reverse complement
caccttgaaccgcatgaactccttgatcactgcctcgcccttgctcacggatccaccaccgccagatccgccgccaccggacccaccaccgcccgagccaccgccaccccctgtggagagaaaggcaaagtggatgtcagtaagaccaatagccggtgatgcggcactcgatctc

SEQ ID NO: 75 - DNA, 97 bp, synthetic construct
  source 1..97: mol_type = other DNA; note = Part of mScarlet minicircle - forward strand
acaagggcggcggtggatccggtggcggcggatctggcggtggaggttcgggtggcggtgggtcgggcggaggtaagtatcaattagaagacgaatt

SEQ ID NO: 76 - DNA, 97 bp, synthetic construct
  source 1..97: mol_type = other DNA; note = Part of mScarlet minicircle - reverse complement
aattcgtcttctaattgatacttacctccgcccgacccaccgccacccgaacctccaccgccagatccgccgccaccggatccaccgccgcccttgt

(19) United States
(12) Patent Application Publication
(10) Pub. No.: US 2024/0401027 A1
(43) Pub. Date: Dec. 5, 2024

(54) METHOD FOR IMPROVED INTRON TAGGING AND AUTOMATED CLONE RECOGNITION

(71) Applicant: PROXYGEN GMBH, Vienna (AT)

(72) Inventors: Andreas REICHER, Wien (AT); Stefan KUBICEK, Wien (AT)

(73) Assignee: PROXYGEN GMBH, Vienna (AT)

(21) Appl. No.: 18/695,539

(22) PCT Filed: Sep. 27, 2022
(86) PCT No.: PCT/EP2022/076866

§ 371 Date: Mar. 26, 2024

(30) Foreign Application Priority Data
Sep. 27, 2021 (EP) 21199270.6
Sep. 28, 2021 (EP) 21199617.8

(51) Int. Cl. C12N 15/10

(52) U.S. Cl. CPC C12N 15/1065

(57) ABSTRACT

The present invention relates to a method for obtaining an intron-tagged pool of cells representing and/or comprising tagged introns. Furthermore, the present invention relates to a pool of cells that may be obtainable by said method, in particular an intron-tagged pool of cells. The present invention furthermore provides for a method for automated recognition of the identity of the tagged intron(s) comprised in the genome of an intron-tagged cell within an intron-tagged pool of cells and a method for assessing the effect of a perturbation on the proteome and/or gene expression levels of an intron-tagged cell within an intron-tagged pool of cells.

The present invention relates to a method for obtaining an intron-tagged pool of cells representing and/or comprising tagged introns. Furthermore, the present invention relates to a pool of cells that may be obtainable by said method, in particular an intron-tagged pool of cells. The present invention furthermore provides for a method for automated recognition of the identity of the tagged intron(s) comprised in the genome of an intron-tagged cell within an intron-tagged pool of cells and a method for assessing the effect of a perturbation on the proteome and/or gene expression levels of an intron-tagged cell within an intron-tagged pool of cells.

While most currently available pharmacological agents, including small molecule pharmaceuticals or pharmacologically active biologics act as inhibitors of enzymes or as modulators of receptors and transporter, drugs may also exert other functions, like (but not limited to) the inhibition or induction of protein-protein interactions and the stabilization or degradation of target proteins.

Currently available methods for the unbiased discovery and for the elucidation of biological/pharmacological functions of bioactive compounds (“mechanisms of action”) and/or some of the known screening methods of substances for their potential use as pharmaceuticals are based on the monitoring of the biological and/or pharmacological effects on proteomes and transcriptomes; see, inter alia, Rix (2009) Nat Chem Biol 5, 616-24; Martinez Molina (2013) Science 341, 84-7; Savitski (2014) Science 346, 1255784; Drewes (2015) Trends Biotechnol 36, 1275-1286; Huber (2015). Nat Methods 12, 1055-7; Subramanian (2017). Cell 171, 1437-1452 e17; or Lamb (2006) Science 313, 1929-35.

Yet, costs and sample preparation requirements associated with these methods preclude their application in large scale screenings and/or they preclude the use of these methods on a large number of drugs/drug candidates at multiple concentrations and/or time points of assessment. Furthermore, other high-content screening approaches that monitor drug effects on cell morphology, as disclosed in Bray (2016) Nat Protoc 11, 1757-74 and/or protein localization approaches by microscopy, for example by staining or fluorescent-tagging approaches, are hampered by the fact that these methods merely allow the monitoring of one or of only a few selected proteins.

The prior art saw in this context approaches in which fluorescently tagged reporter cells are generated either by overexpression to non-physiologic levels, or by targeting a single gene with a homologous recombination template. Also “genetrap” approaches have been applied in this context; see, e.g. Morin (2001) Proc Natl Acad Sci USA 98, 15050-5). Yet, such approaches are limited by integration site biases. Moreover, these “genetrap virus approaches” employ viral constructs in order to generate tagged cell pools. Since the employed viruses have tremendous integration site biases, namely in the first intron, some genes are targeted much more efficiently by these viral constructs than others. Furthermore, there are no means in these approaches to select specific gene sets or specific introns to be targeted.

Serebrenik and colleagues proposed a tagging technology of selected endogenous genes by homology-independent intron targeting, whereby intron-based protein trapping with homology-independent repair-based integration of a generic donor was combined, see Serebrenik (2019) Genome Research 29, 1322-28. The corresponding approach is based on homology-independent CRISPR-Cas9 editing to place a fluorescent tag as a synthetic exon into introns of individual target genes by combining a generic sgRNA (single guide RNA, also referred to as gRNA) excising a fluorescent tag flanked by splice acceptor and donor sites from a generic donor plasmid with co-expression of a gene-specific intron-targeting sgRNA. Based on the fact that this technology employs generic donors, it is speculated that this technology would enable the generation of multiple fusion cell lines but that this would require the cloning of additional intron-targeting sgRNAs. Yet, from the technology as provided by Serebrenik, an efficient way to determine which cell expresses which protein is not feasible since there is no way to establish a direct readout for the respective genomic target locus that is targeted.

Reicher (2020) Genome Res 30, 1846-1855 and WO 2021/099273 (incorporated herein by reference) provided an improved tagging technology, in particular regarding the tagging of single introns (intron frames/intron phases) of multiple genes with one single tag. However, computer-assisted assessment or more detailed analysis of the tagging events in a plurality of tagged cells is limited and needs improvement over the methods as provided by Reicher (2020) loc. cit.

Accordingly, there is a need in the art to provide for improved means and methods for a characterization of expressed proteins or of factors influencing individual proteins, including their expression and/or cellular localization in whole proteome analysis approaches, inter alia, in computer-assisted approaches.

The technical problem is solved by the embodiments as characterized in the claims and as provided herein.

Accordingly, the present invention in particular relates to a method for obtaining an intron-tagged pool of cells representing and/or comprising tagged introns, said method comprising the steps of:
(a) selecting introns to be targeted in the genome of a cell;
(b) identifying guide RNA (gRNA) sequences suitable for inserting a tag sequence in the selected introns in the genome of a cell to be intron tagged;
(c) cloning identified gRNA sequences and tag sequence into transfection and/or transduction vectors;
(d) contacting a population of the cells to be intron-tagged with said transfection and/or transduction vectors of (c);
(e) selecting of intron-tagged cells based on the presence of the tag sequence; and
(f) obtaining a pool of cells representing a plurality of tagged introns
wherein steps (b) to (e) are repeatedly performed using a unique combination of the gRNA sequences and the tag sequence for each round of repetition.

The present invention further relates to the generation of a pool of cells comprising cells with multiple tagged introns and inter alia the possibility for automated clone recognition as detailed herein. The present invention also provides for a novel intron tagged cell pool (i.e. a pool of cells) that is characterized by multiple individual gene tagging events per cell.

A CRISPR/Cas9 based intron targeting approach can be used for generating highly diverse pools of cells, wherein in every cell a different gene is tagged (see Example 2 herein) in accordance with this invention and as also disclosed in WO 2021/099273 or in Reicher (2020) Genome Res 30, 1846-1855. Genes within a cell can be tagged in exonic and/or intronic regions, but intronic regions are generally preferred in context of this invention. Furthermore, in context of this invention the terms “intron-tagging” relates in particular to the marking of an expressed gene with a “tag” or “label”, whereby said tag/label is preferably a fluorescence label. Whereas said label could be introduced in any part of the expressed gene, for example a the N- or C-terminus, said “tag”/“label” can also be introduced within the expressed amino acid sequence. In other words, by “intron-tagging” as used herein is meant the introduction of a “tag”/“label” in frame with a preceding and/or a following exon sequence without introduction of frameshifts or premature stop codons such that the resulting open reading frame can be translated into the corresponding fusion protein. In context of this invention the terms “intron-frame” and “intron-phase” are used interchangeably.

Accordingly, in context of this invention it is envisaged to “tag” genes in introns and/or exons (preferably in introns) and at one or more positions such as at the beginning, at the end or within a genomic sequence spanned by a gene, such that the fusion protein expressed from the endogenous promoter contains the expressed tag sequence at those positions. Those tagged pools of cells can be exposed to various perturbations, e.g. environmental factors, drug treatments/exposure. Time-lapse microscopy can be used to follow changes in protein abundance or subcellular protein localizations of any of the expressed tagged genes (via “intron-tags) in the pool of cells. For identification of the tagged genes in responding cells in the pool of cells, in situ sequencing of the intron-targeting sgRNA indicating the identity of the tagged intron/gene was necessary in earlier approaches. This additional step to identify clones can limit the throughput when using the pool of cells in screening applications, for example when using a pool of cells representing hundreds to thousands of different tagged genes and exposing that pool of cells to hundreds to thousands of drug compounds to either profile and characterize existing drugs or screen for compounds that alter subcellular protein localizations or degrade or stabilize target proteins. The limitation in throughput is particularly due to the additional rounds of imaging as part of the in situ sequencing. Accordingly, previously a single well containing thousands of different tagged genes had to be imaged an additional 8 times to determine the first 8 bases of the intron-targeting sgRNA in every cell (given the need to read 8 bases to unambiguously assign sgRNA identity from all the sequences present in a given sgRNA library). Furthermore, after time-lapse fluorescence microscopy, cells had to be fixed in the well and further processed before the in situ sequencing could be done, requiring additional reagents and potentially additional liquid handling equipment for processing hundreds of wells when looking at hundreds of perturbations.

Identification of the clones in the pool of cells (i.e., the identity of the respectively tagged genes within the cell) using only image analysis, without in situ sequencing is so far not possible in a pool of cells with only one tagged protein per cell as detailed in WO 2021/099273 or in Reicher (2020) Genome Res 30, 1846-1855. This is because there are almost no proteins with a very specific and unique subcellular localization and intensity pattern, e.g. most proteins with a mitochondrial localization cannot be discriminated from one another.

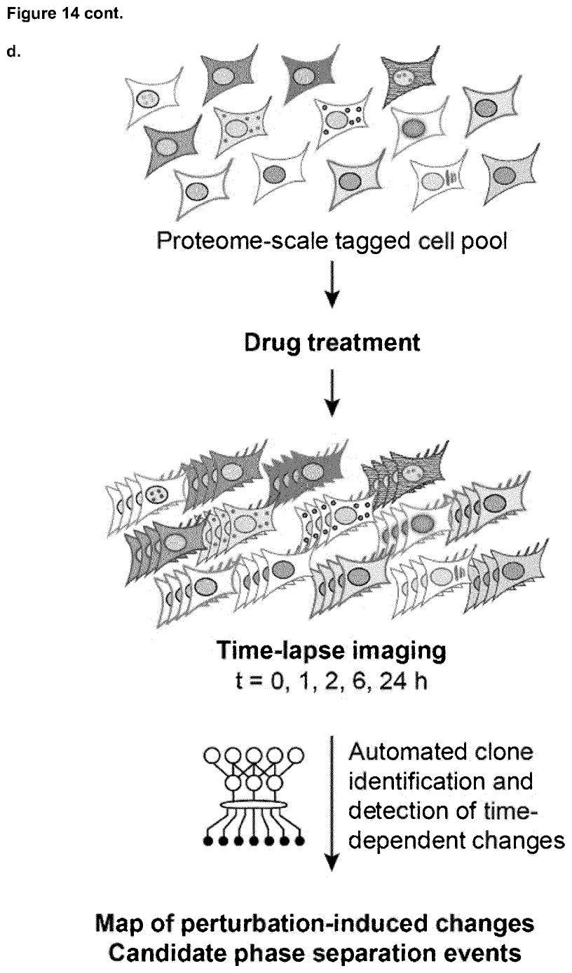

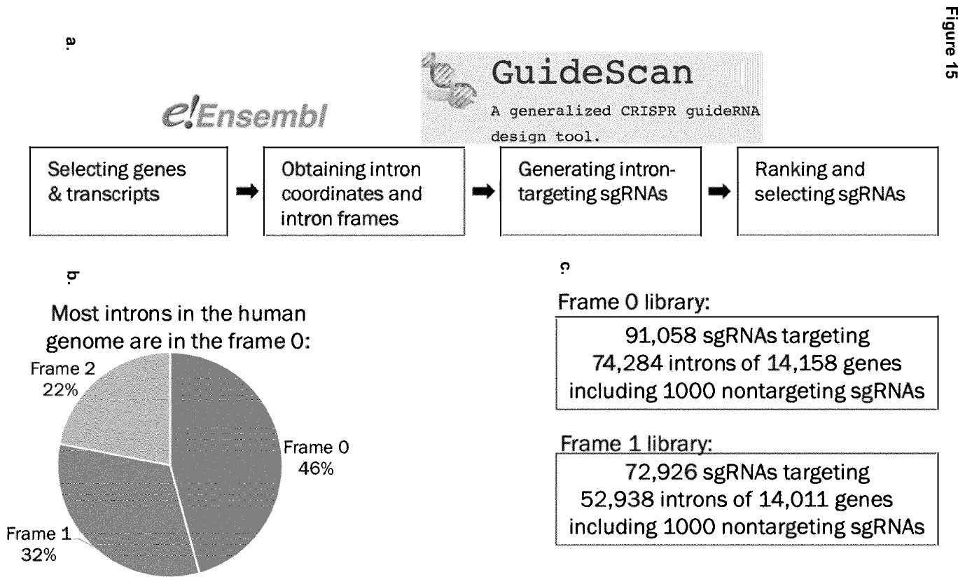

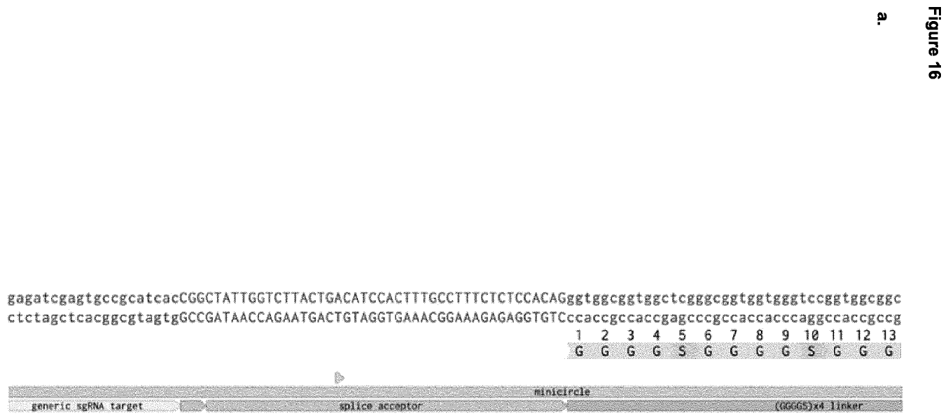

As described herein, the inventors have developed a protocol for the generation of a pool of cells, in which clones in the pool of cells expressing (multiple) different (endogenously) intron-tagged genes can be identified. One of the advantages of this invention is that with the herein provided means and methods, also and for example computational/automated-assisted analysis of cells/clones, in particular analysis of the presence or identity of intron-tagged genes in the cells/clones, for example by the analysis of images, in particular fluorescent microscopy images, is now possible even without the need to perform in situ sequencing. Thus, the present invention also provides for an advantageous “automated clone recognition”. Such an “automated clone recognition” is enabled in accordance with the present invention by, inter alia, tagging multiple proteins per cell being expressed in particular from their endogenous promoters using multiple rounds of intron tagging with intron-targeting sgRNA libraries targeting different intron frames per round (also called intron phases) and constructs for splice acceptor/donor flanked fluorescent proteins of different colours in the different reading frames (see, e.g., FIG. 14a, 14b). Per each round of tagging, a single gRNA sequence comprised in the gRNA library targeting one specific intron frame is integrated into the genome of the cell in order to enable the subsequent tagging step. This is, in accordance with this invention, inter alia achieved by adjusting the ratio defined by the number of transfection/transduction vectors to be used and the number of cells to be transfected/transduced. The person skilled in the art is readily aware of how such a ratio can be controlled and implemented. The aforementioned ratio is generally to be chosen in a way that it is smaller than 1. When lentiviral vectors encoding the gRNAs are to be used, a low MOI may be used for transduction, preferably a MOI of 0.05. Further information is provided herein below. In other words, in accordance with the present invention merely a single gRNA sequence is to be incorporated into the genome of an individual cell per tagging round. Therefore, it is preferred in accordance with the present invention that the proportion of incorporated gRNA sequences (i.e., gRNA sequence identity as defined by its unique nucleotide sequence) to incorporated tag sequence per tagging round is one. If this were not the case, i.e. more than one like 2, 3, 4, 5, 6, 7, 8, 9, 10, 50, 100, 500, 1000 and the like gRNAs would be incorporated into the genome of an individual cell per tagging round, this proportion would be greater than one which would prohibit the direct assignment of the tagged intron to exactly one single gRNA as multiple gRNAs comprised in the gRNA library, which may comprise up to tens of thousands of individual gRNA sequences (see, e.g. Example 15), being used for one round of tagging, would be present in one cell.

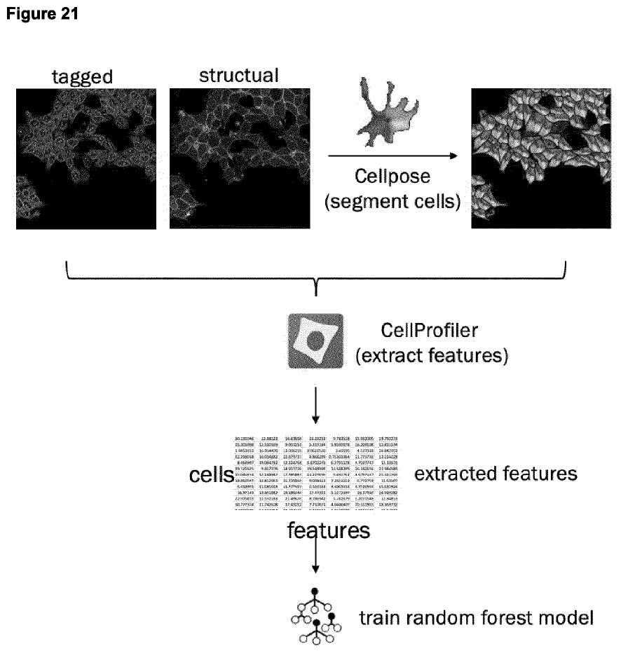

As illustrated in the appended examples, the inventors have successfully prepared an intron-tagged pool of cells as provided herein, by isolating about 11,000 inventive intron-tagged cells (i.e. representing a pool of intron-tagged cells wherein an individual cell in said intron-tagged pool of cells is characterized by at least two different tags in at least two different intron frames/phases of at least two different genes). Furthermore, the inventors isolated hundreds, e.g. about 2000, clones from such an intron-tagged pool of cells. Furthermore, as also illustrated in the appended examples, the inventors have successfully trained a computational model that can recognize those clones in the pool of cells (see, e.g. FIG. 14c, 14d and FIG. 21). The inventors have surprisingly found, as illustrated herein, that this recognition was now possible by a computer-assisted analysis of microscopic images, analyzing the emitted signal of expressed (fluorescent) tags within the isolated clones. In accordance with this invention, the isolation of clones for training a computational model or algorithm, however, only has to be done once for every pool of cells. This is a clear advantage over the prior art.

The improved tagging strategy described herein in context of this invention by using multiple independent sgRNA libraries for different intron reading frames (or phases), each with a matching fluorophore construct/tag is far superior to the strategy of using the same sgRNA library multiple times with different fluorophores. In other words, the invention as provided herein relates to a novel method for obtaining an intron-tagged pool of cells representing and/or comprising tagged introns, said method comprising the steps of:
(a) selecting introns to be targeted in the genome of a cell;
(b) identifying guide RNA (gRNA) sequences suitable for inserting a tag sequence in the selected introns in the genome of a cell to be intron tagged;
(c) cloning identified gRNA sequences and tag sequence into transfection and/or transduction vectors;
(d) contacting a population of the cells to be intron-tagged with said transfection and/or transduction vectors of (c);
(e) selecting of intron-tagged cells based on the presence of the tag sequence; and
(f) obtaining a pool of cells representing a plurality of tagged introns
wherein steps (b) to (e) are repeatedly performed using a unique combination of the gRNA sequences and the tag sequence for each round of repetition.

Preferably, upon performing the first round of said steps (a) to (e), a pool of cells, i.e. an intron-tagged pool of cells, is obtained, wherein essentially each cell in the pool comprises a tagged intron that is tagged with the tag sequence employed in said first round.

Preferably, in context of the present invention, an intron relates to the intron of an endogenous gene, i.e., the introns are endogenously tagged.

Furthermore, between said steps (d) and (e), at least one of the following steps (d′), (d″) and (d′″) may be performed as described herein and as illustrated in the appended Examples. In particular, said step (d′) may comprise introducing into the population of cells a vector/plasmid that acts as a generic donor plasmid that provides (i.e comprises) the tag sequence, for example an EGFP sequence, to be integrated into the introns, preferably by means of transfection, as described herein. This generic donor plasmid may contain a cutting enzyme, e.g. Cas9, cut-site (targeted by a generic sgRNA sequence that may be present, e.g., on a Cas9 plasmid), a splice acceptor site, tag sequence (e.g. EGFP), a splice donor site and another Cas9-cut site (targeted by the generic sgRNA sequence that may be present, e.g., on a Cas9 plasmid).

Furthermore, said step (d″) may comprise introducing into the population of cells a vector/plasmid that encodes a generic sgRNA/gRNA, in particular for excising the tag sequence flanked by the splice acceptor and donor sites from the generic donor plasmid, as described herein.

Furthermore, said step (d″) may comprise introducing into the population of cells a vector/plasmid that encodes an enzyme that cuts DNA (e.g. genomic DNA or a plasmid/vector) at a location defined by one or more gRNA(s) in the cell, such as Cas9, Cpf1 or Cas12b.

Preferably, said steps (d′), (d″) and (d″) are performed simultaneously. Preferably, in steps (d′), (d″) and/or (d′ ″), the vectors are introduced transiently, in particular by means of transfection. Furthermore, the generic gRNA and the cutting enzyme, e.g., Cas9, may be encoded in the same vector/plasmid, as described herein and as illustrated in the appended Examples.

Furthermore, in step (e) of a certain round of repetition, the intron-tagged cells are, in particular, selected based on the presence of the specific tag sequence employed in the corresponding round of repetition. As mentioned above, in each round of repetition, a unique combination of gRNA sequences and tag sequence is employed. This further means, in particular, that the gRNA sequences and tag sequences are different between the different rounds.

Furthermore, in each round of repetition, preferably a different intron frame (i.e., intron frame 0, 1 or 2) is tagged, in particular, by employing in a certain round of repetition gRNA sequences that are suitable for inserting a corresponding tag sequence into introns having a certain corresponding intron frame.

Preferably, in said step (f) a pool of cells is obtained, wherein essentially each cell in the pool of cells comprises at least two tagged introns, preferably at least two tagged introns having different intron frames (the number depending on the number of rounds of repetition), and wherein essentially each of the tagged introns within a cell is tagged with a different tag sequence, i.e., the specific tag sequence employed in the corresponding round of repetition.

Accordingly, in said step (f) preferably a pool of cells is obtained, wherein essentially each cell in the pool of cells comprises at least two tagged introns, and wherein essentially each of the tagged introns per cell has a different intron frame and is tagged with a different tag sequence. As further described herein, introns having the same intron frame are preferably tagged by the same tag sequence, and introns having different intron frames are preferably tagged by different tag sequences.

In particular, a cell, preferably essentially each cell, in the inventive pool of cells provided herein may comprise two different tags/tag sequences in two different intron frames of two different genes (i.e. in two introns of two different genes, wherein the two introns have different intron frames), or three different tags/tag sequences in three different intron frames of three different genes (i.e. in three introns of three different genes, wherein each of the three introns has a different intron frame). Preferably, essentially each clone of cells in the inventive pool of cells provided herein comprises a unique combination of tagged introns/fusion proteins, i.e., a combination of tagged introns/fusion proteins that is different from essentially every other cell in the pool (belonging to other cell clones). In particular, as used herein, essentially all cells of a clone of cells comprise the same combination of tags including the same tagged introns.

Furthermore, in context of the present invention, the presence of a certain label/tag/tag sequence as described herein (e.g. a fluorescent protein such as EGFP) in a cell can be linked to the presence of a corresponding sgRNA in the cell and accordingly also to the presence of the corresponding tagged intron which is translated into a fusion protein comprising said tag, as described herein. This is advantageous, at least, because the identity of the tagged introns/fusion proteins in a cell can be easily and robustly determined, e.g., by determining the gRNAs contained in the cell, as described herein.

As described herein, the present invention further relates to a pool of cells which corresponds to a pool of cells obtained or obtainable by the inventive method for obtaining an intron-tagged pool of cells provided herein. Yet, the pool of cells of the present invention may be also obtainable by other methods, e.g., methods yet to be developed. Advantageously, the inventive pool of cells provided herein further enables or facilitates the automated clone recognition of the invention described herein.

Targeting the same intron frame in two consecutive rounds of tagging would not be successful in the context of the present invention since during the second round of tagging, two intron-targeting sgRNAs are (or would be) present in the cell. Therefore, the second fluorophore construct could also integrate at the target site of the first intron-targeting sgRNA, if there are still unedited alleles available which is the case when performing those experiments in cells that are not fully haploid, but rather diploid or polyploid; or in which the gRNA target sequence remained intact after the first editing round. As provided in context of the present invention, this disadvantage is overcome by using libraries, in particular gRNA libraries, targeting different frames and matching tag sequences (e.g. fluorophore constructs) per tagging round. For example, the frame1 mScarlet construct introduced in the second round of tagging, cannot lead to tagging of a target gene of a frame 0 intron-targeting sgRNA, because integration of the construct at such a site would result in a frameshift and expression of non-functional and non-fluorescent proteins. As described above, using this inventive tagging strategy, every one of the at least two tagged genes (see e.g., FIG. 14a) in every clone can be easily assigned to one of the at least two intron-targeting sgRNAs in every clone, e.g. the gene tagged with the e.g. fluorescent tag like (E)GFP frame 0 minicircle is indicated by the intron-targeting sgRNA belonging to the frame 0 library.

With the technology as provided herein, it is now not only feasible to obtain an intron tagged pool of cells wherein the individual cells in this pool comprise at least two (i.e., multiple) tags. Besides this novel intron-tagged pool of cells that was not described in Reicher (2020) Genome Res 30, 1846-1855 loc cit, the technology provided herein, as illustrated in the Examples 4-8, enables for the first time a computer-assisted recognition of tagged introns in the genome of an intron tagged cell within the novel intron-tagged pool of cells as obtained by the novel technology provided herein which is in particular based on a repetitive performance of the steps b-e, as described herein above, wherein consecutive rounds of tagging are performed and wherein each round uses a unique combination of(s) gRNA sequences and a tag sequence for each round of repetition. In other words, each tagging round is characterized by unique(s) gRNA sequences, in particular, unique libraries of(s) gRNA sequences and a corresponding individual tag for these unique libraries, for example an individual fluorescent tag, like for example a unique library of(s) gRNA sequences based on selected introns is employed with a first fluorescent tag like, e.g., a green fluorescent tag like (E)GFP, the second round in the repetition cycle uses another unique library of(s) gRNAs and another fluorescent tag like, e.g., a red fluorescent tag like mScarlet (Bindels (2017) Nat Methods, 14, 53-56). The person skilled in the art readily understands that also other “tags”/labels” can be used as long as these “tags”/“labels” are individual “tags”/“labels” for unique gRNA libraries and that for each round of repetition within the above recited method for obtaining (an) intron-tagged pool(s) of cells a different “tag”/“label” (preferably fluorescent “tags”/“labels”) is employed. With the technology provided herein, it is, therefore, possible to assess for example via automated clone recognition and/or computer means the identity of the (individually) tagged introns comprised in the genome of an intron-tagged cell within the intron tagged pool of cells.

Also, with the intron tagged pool of cells of the present invention and comprising individual(s) gRNAs from the used(s) gRNA libraries and a corresponding individual tag for each of these libraries, it is now also possible to screen such intron tagged pools of cells for environmental factors like, e.g. the influence of drugs or medicaments. Accordingly, the intron tagged pool of cells as obtainable/obtained by the methods described in this invention can be used e.g. for drug screening or assessment of e.g. environmental factors on cells (like, e.g. assessment of toxic compounds etc).

The intron tagged cell pools can also be used in the assessment of effects caused by perturbations of the proteome and/or of gene expression levels (i.e., on the mRNA level).

Accordingly, within the present invention, multiple intron tagging rounds are performed using a unique combination of the gRNA sequences (i.e., a gRNA library as is exemplified in Example 4) and an associated individual tag sequence. Accordingly, the term “unique combination of the gRNA sequences and the tag sequence for each round of repetition” as used herein is to be understood as using such a unique combination for each round of repetition, i.e. for round one a gRNA library targeting intron frame 0 may be used in combination with a first tag like a green fluorescent GFP tag for example, whereas for round two a different gRNA library targeting intron frame 1 (i.e., a different intron frame that has not been used in a previous round of tagging) may be used in combination with a second tag like a red fluorescent mScarlet tag as illustrated, inter alia, in appended Example 6.

As shown in the appended examples, the methods of the present invention comprise, inter alia, a step of identifying gRNA sequences suitable for inserting a tag in introns in the genome of a cell. In the methods of the present invention, it is preferred that a cell comprised in a population of cells that is to be intron tagged receives multiple tags. The general principle of single intron tagging is described by Serebrenik et al. (2019), loc cit. and in WO 2021/099273. The strategy of Serebrenik et al. relies on a single generic sgRNA excising a single fluorescent tag flanked by splice acceptor and donor sites from a generic donor plasmid, which is co-expressed with a single gene-specific intron-targeting sgRNA specifying the single integration site. The strategy in WO 2021/099273 relies on tagging only one intron per cell using a gRNA library targeting one intron frame/phase of a library of genes in combination with a single “tag”/“label”, in particular a single fluorescent tag/label.

In contrast, as shown in the appended examples, the means and methods of the present invention lead, inter alia, to the generation of (an) intron-tagged cell pool(s) wherein each cell is intron tagged multiple times in different genes with different fluorescent tags. This is achieved by tagging cells in a population of cells of the same cell type, whereby each cell receives multiple tags/labels. In particular, the population, e.g., the pool of cells according to the invention, thus comprises or essentially consists of cells tagged multiple times at different genomic sites with different tags/labels that are actively transcribed from their endogenous promoters, providing as a whole a tagged proteome or tagged parts thereof. Accordingly, and in contrast to the technology as provided by Serebrenik et al. (2019), loc cit. and as provided in WO 2021/099273, the present invention provides for means and methods wherein the whole proteome (or at least substantial parts thereof) can be automatically monitored in an intron-tagged cell population comprising cells with multiple intron tags. Therefore, the present invention provides for an automated “one shot” analysis of the whole proteome (or substantial parts thereof). As such, the present invention, for the first time, allows the automated analysis of the whole proteome (or at least substantial parts thereof) in one experiment by using different gRNA libraries, each of them targeting a different intron frame/phase for a multitude of introns to be tagged in combination with corresponding fluorescent tags wherein the detectable signal of a given fluorescent tag emitted from an intron tagged cell is indicative of the corresponding pools of introns to be tagged by the corresponding gRNA library. In order to establish automated clone analysis of the intron-tagged pool of cells, fluorescence microscopy is to be combined with in situ sequencing in order to train a model of a computer vision algorithm. In this regard, the inventors found that the use of a sequencing-enabling vector that expresses the gRNA as part of the transcript that can be detected by in situ sequencing, such as a CROPseq vector, as a transduction vector allows the identification of the individual gRNA sequence, which corresponds to the tagged protein in each clone in the pool, identifiable e.g. by using an imaging technique such as microscopy or FACS.

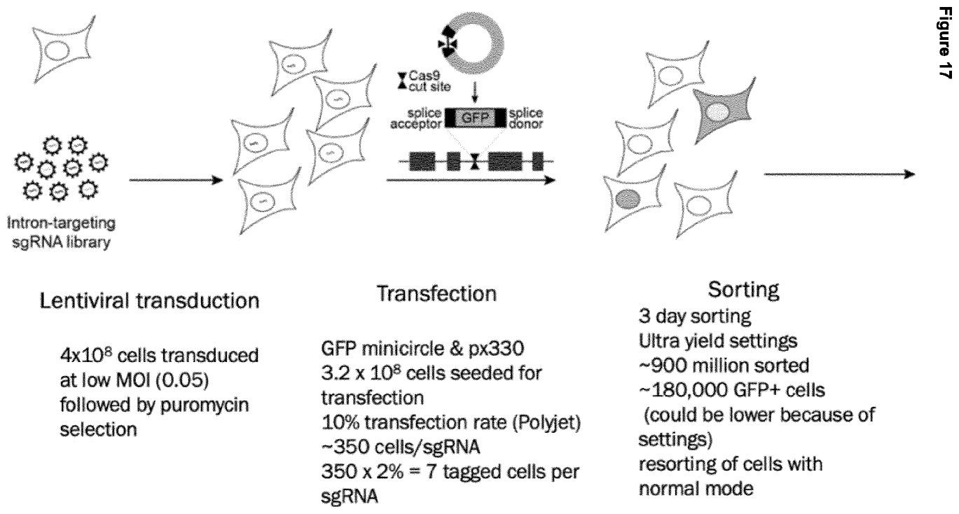

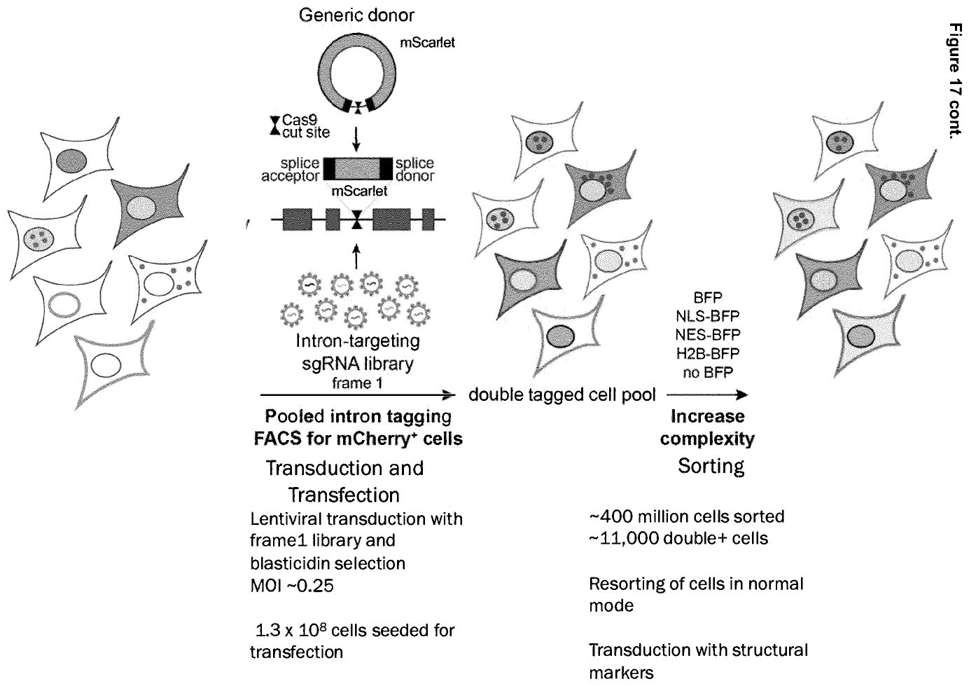

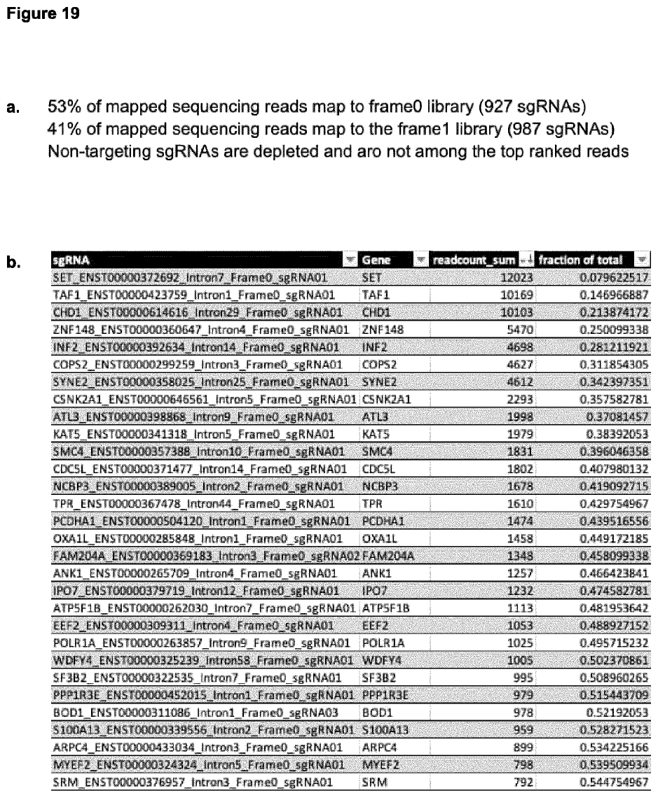

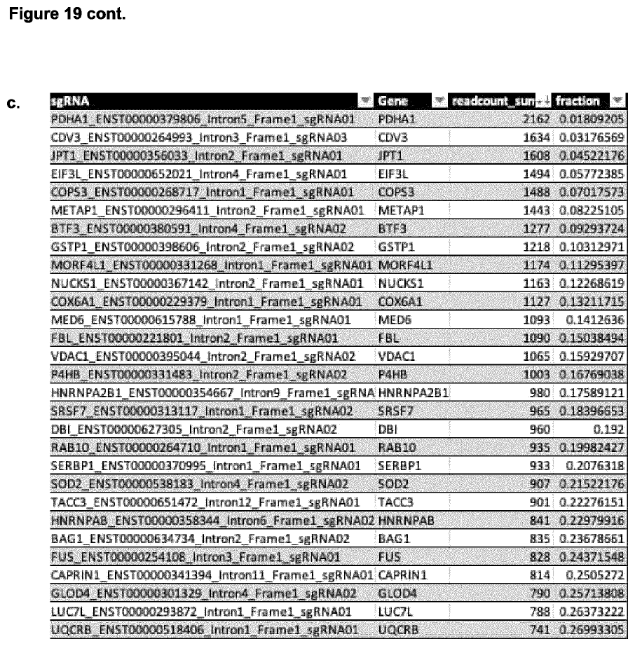

While most currently available pharmacological agents, including small molecule pharmaceuticals or pharmacologically active biologics act as inhibitors of enzymes or as modulators of receptors and transporters, drugs may also exert other functions, like (but not limited to) the inhibition or induction of protein-protein interactions and the stabilization or degradation of target proteins. In context of this invention, a scalable automated strategy to discover in real time the effects drugs exert on levels and subcellular localizations of a large subset of the proteome is provided. Illustratively for the present invention, CRISPR-Cas9 based intron tagging was employed to generate cell pools expressing thousands of GFP/mScarlet double positive cells, translating into 927 GFP and 987 mScarlet tagged (see, e.g. FIGS. 17 and 19a)-fusion proteins as identified by in situ sequencing. This is also documented in the appended figures and examples. From the pool of tag-double positive cells (here illustratively GFP/mScarlet double positive cells), more than 2000 individual clones were isolated and these double positive cells where genotyped and analyzed/imaged by fluorescence microscopy in order to establish a labeled dataset for training a computational model for establishing computer vision to automatically recognize the identity of the tagged introns comprised in the genome of an intron-tagged cell within an intron-tagged pool of cells. After training, the model was used to predict the identity of the clones in test images that were not employed for training of the model. It was found that the clones were correctly recognized with an accuracy of 98%. It is envisaged that the means and methods of the invention further allow to recognize clones within a pool of cells, e.g. an microscopic image of a pool of cells, with a similarly high accuracy, e.g. of about 90%, about 95% or about 98%. Evidently, the accuracy may be a bit lower when a high number of different clones is to be recognized and/or discriminated in a pool of cells. Thus, also a somewhat lower accuracy of, e.g., at least 70%, at least 80%, or at least 90% may still indicate an excellent performance, in particular when the pool of cells comprises many different clones e.g. several thousands of different clones. Furthermore, the inventive pool of cells in combination with automated clone recognition to recognize the identity of such cells (i.e., the identity of the tagged introns comprised in those cells) may also be used to study protein dynamics in response to various metabolic perturbations either in an arrayed or pooled format and strategies to identify individual drug-responsive clones in the pool are provided.

Thus, in accordance with the present invention a pool of cells representing (i.e. comprising) a plurality of tagged introns can be obtained in step f) of the method for obtaining an intron-tagged pool of cells representing and/or comprising tagged introns as detailed herein above. A pool of cells of the present invention may comprise at least 2, at least 3, at least 4, at least 5, at least 6, at least 7, at least 8, at least 9, at least 10, at least 20, at least 50, at least 100, at least 1000, at least 10000, at least 100000, at least 1000000, at least 10000000, at least 100000000, at least 1000000000, at least 10000000000 cells comprising tagged introns. Furthermore, these cells comprising tagged introns may belong to hundreds or thousands of clones in the pool of cells. Thus, a pool of cells of the present invention may comprise at least 2, at least 3, at least 4, at least 5, at least 6, at least 7, at least 8, at least 9, at least 10, at least 20, at least 50, at least 100, at least 1000, at least 2000, at least 3000, at least 4000, at least 5000 or at least 10000 clones of cells. This is meant by “plurality” in context of step f) in context herein above. For example, as shown in illustrative Example 6 and FIG. 17 about 180000 single and 11000 double tagged cells were sorted after one or two rounds of intron tagging respectively by the inventors. Furthermore, about 2000 clones of cells comprising two tagged introns per cell were isolated and employed for automated clone recognition as described herein. Furthermore, a “plurality” of tagged introns within the meaning of the present invention is to be understood as the entirety of individual introns that are tagged in the entirety of cells being comprised in the pool of cells comprising the tagged introns. Therefore, in accordance with the means and methods of the present invention, a plurality of tagged introns may comprise at least 2, 3, 4, 5, 6, 7, 8, 9, 10, 15, 20, 25, 30, 35, 40, 45, 50, 60, 100, 200, 500, 1000, 1500, 2000, 2500, 3000, 5000, 10000, 20000, 50000, 100000, 200000, 500000 introns. A plurality of e.g. 10 introns may for example already be useful for certain applications.

For example, the pool of cells of the invention may comprise a plurality of cell clones, e.g., about 1000 clones, wherein essentially each cell (or clone of cells) in the pool comprises two tagged introns having different intron frames tagged with different tags/labels (e.g. an GFP tagged frame 0 intron and an mScarlet tagged frame 1 intron), and wherein essentially each cell clone in the pool is characterized by a unique combination of tagged introns and/or corresponding fusion proteins. Accordingly, this exemplary pool of cells comprises a plurality of tagged introns, e.g., about 2000 (i.e. two per cell), wherein preferably most or the vast majority of the tagged introns are unique in the pool.

In a preferred embodiment of the method of the present invention for obtaining an intron-tagged pool of cells representing and/or comprising tagged introns, the transfection and/or transduction vectors as recited in step (d) comprise gRNA sequences to be integrated into the genome of the transfected and/or transduced cells. It is preferred that a single gRNA sequence is integrated into the genome of the individual transfected and/or transduced cell within the population of the transfected and/or transduced cells. This is, in accordance with the present invention, in particular achieved by adapting the ratio of cells to be transfected to the number of transfection and/or transduction vector molecules (i.e. the size of a given gRNA library), i.e., for (lentiviral) transduction vectors, a MOI (multiplicity of infection) of 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5 or any MOI value in between may be used, preferably a MOI of 0.05. Of note, this is envisaged in accordance with this invention in order to ensure a sufficient gRNA library coverage in the pool of cells having received gRNA encoding vectors.

Furthermore in the above recited method for obtaining an intron-tagged pool of cells representing and/or comprising tagged introns, the transfection and/or transduction vectors (comprising said tag sequence) are capable of integrating the tag sequence into the genome of the transfected and/or transduced cells in step (d) of said method. Accordingly, introns or exons which are actively transcribed from the endogenous promoters may be targeted at various sites spanning the genomic sequence of a gene, but generally introns are preferred.

In particular, the cells to be (intron)-tagged further comprise an enzyme for integrating the tag sequence(s), in particular an enzyme that cuts DNA (e.g. genomic DNA and/or plasmid/vector DNA) at a location defined by the gRNA(s) in the cell such as Cas9, Cpf1 or Cas12b. Preferably said enzyme is Cas9. Said enzyme, e.g., Cas9, may be integrated into the cells to be tagged, preferably transiently, for example, by introducing the nucleotide sequence encoding the enzyme (e.g. Cas9), e.g. by means of transfection or transduction, into the cells. Introduction of said enzyme, e.g. Cas9, may be performed, for example, before, simultaneously with or after (preferably simultaneously with or after) contacting the population of the cells to be intron-tagged with the transfection and/or transduction vectors encoding the identified gRNA sequences, i.e. the gRNA library. Preferably, said enzyme, e.g. Cas9, (in particular, a nucleic acid molecule/vector encoding said enzyme) is transfected simultaneously with the transfection vector containing the tag sequence and/or the vector/plasmid encoding the generic gRNA. Furthermore, said enzyme, e.g. Cas9, is preferably provided transiently to the cells (rather than being stably integrated into the genome). It is also possible that the transfection and/or transduction vectors employed in step (d) of the inventive method for obtaining an intron-tagged pool of cells provided herein, and/or a transfection or transduction vector encoding a generic gRNA for cutting the vector(s) containing a tag sequence, further encode said enzyme, e.g., Cas9.

The intron-tagged cells may be selected (or separated from cells that do not comprise the desired intron-tags) based on the emitted signal of the expressed protein tag being emitted from a given cell. Such a signal may be used for cell isolation based on methods such as fluorescence-activated cell sorting (FACS). Accordingly, such a separation or selection may be achieved by routine methods, like cell sorting, e.g. by FACS sorting.

The inventive pool of cells (i.e., the pool of intron tagged cells) as obtainable by the means and methods provided herein may contain at least 2, at least 5, at least 10, at least 20, at least 50, at least 100, at least 200, at least 300, at least 400, at least 500, at least 1000, at least 1500, at least 2000, at least 2500, at least 3000, at least 10000, or at least 20000 tagged introns.

In a preferred embodiment of the method for obtaining an intron-tagged pool of cells representing and/or comprising tagged introns, said (intron) tagging is repeatedly performed. i.e at least 2 times, at least 3 times, at least 4 times, at least 5 times or at least 6 times. For example, the intron tagging may be repeatedly performed two or three times, in particular for two or three intron frames, respectively. Again, in accordance with this invention, each “repetition” is carried out using a unique combination of the gRNA sequences and an individual tag sequence/label sequence for each individual round of repetition. Accordingly, the tag/label for each round of repetition is a different (fluorescent) tag/label. Corresponding tags or labels are well-known in the art. Herein and in context of the invention, tags or labels may comprise, but are not limited to, fluorescent tags/labels like GFP, EGFP, YFP, RFP, mScarlet or BFP. Said tag/label may also be selected from a tag/label suitable for detection by covalent (e.g. Halo tag, Clip tag, Snap tag, Spy tag) or non-covalent (e.g. Strep-tag, HA tag, dTag) binding to a detection reagent enabling detection by microscopy, e.g. fluorescence or luminescence. In accordance with this invention, also means and methods are provided wherein cellular structures (like organelles, membranes, nuclei, mitochondria, substructure(s), cytoskeleton, cell membrane, cell wall, chloroplast, endoplasmic reticulum, Golgi apparatus, mitochondrion, nucleus etc.) are also labelled (besides the signals as emitted by the intron-tags/intron-“labels”). The skilled person knows corresponding labeling methods of such cellular structures/substructures/organelles etc. Such methods may comprise, but are not limited to, the use of further fluorescent and/or luminescent marker(s) selected from the group consisting of miRFP670, mAmetrine, (mTag)BFP(2), fluorescently labeled antibodies, DAPI, Hoechst dyes (within this invention comprise but are not limited to i.e. Hoechst 33258, Hoechst 33342 and Hoechst 34580) is/are used to label the cell comprising the tagged-introns after the final round of intron tagging. Accordingly, it is envisaged that in the means and methods for obtaining an intron-tagged pool of cells representing and/or comprising tagged introns as provided herein above that before step (f) (i.e. after the selection step of cells that comprise the desired intron-tags; see step (e)), further fluorescent and/or luminescent marker(s) may be used to label the cell and/or cellular substructures within the individual cell comprising the tagged-introns. Such fluorescent and/or luminescent marker(s) may comprise miRFP670, mAmetrine, (mTag)BFP(2), fluorescently labeled antibodies, DAPI, Hoechst dyes (i.e. Hoechst 33258, Hoechst 33342 and Hoechst 34580) etc.

In accordance with this invention, the gRNA sequences (i.e. the gRNA library) for each round of repetition (i.e., tagging repetition) may target a different one of three intron frames/phases and/or a different one of three exonic open reading frames. It is preferred that said intron frames and/or exonic open reading frames was/were not used in (a) previous round(s) of repetition. Accordingly, the inventive cell pool of the invention may comprise a plurality of intron and/or exon-tagged cells, wherein an individual cell or clone of cells in said pool of cells is characterized by at least two different tags in at least two different intron frames and/or exonic open reading frames of at least two different genes. In particular, a cell or essentially each cell, in said cell pool may comprise 2, 3, 4, 5, or 6 different tags in 2, 3, 4, 5, or 6 intron frames or exonic open reading frames of 2, 3, 4, 5, or 6 different genes, respectively.

In particular, a cell, preferably essentially each cell, in the inventive pool of cells provided herein may comprise two different tags/tag sequences in two different intron frames of two different genes (i.e. in two introns of two different genes, wherein the two introns have different intron frames), or three different tags/tag sequences in three different intron frames of three different genes (i.e. in three introns of three different genes, wherein each of the three introns has a different intron frame).

In the herein provided means and methods for obtaining an intron-tagged pool of cells representing and/or comprising tagged introns, the identified gRNA sequences of step (b) are preferably cloned into a transduction vector and the tag sequence is preferably cloned into a transfection vector. Preferably, said transfection vector allows the production of minicircle DNA. It is further preferred that the transduction vector encoding the sgRNA is a sequencing vector, preferably a CROP-Seq vector.

In the inventive method for obtaining an intron-tagged pool of cells representing and/or comprising tagged introns, the introns to be targeted may be comprised/located in genomic sequences of, e.g. metabolic enzymes, chromatin proteins, kinases, genes coding for proteins in the ubiquitin/proteasome pathways, transcription factors, ion channels, transporters, or receptors. It is envisaged that at least one intron per protein coding gene in the genome is targeted in the inventive method The introns to be targeted may be selected based on the reading frame of the upstream exonic sequence, wherein the sequence to be inserted is in-frame with the exonic sequence.

The gRNA sequences suitable for inserting a tag sequence in the selected introns in the genome of the cell may be identified according to Cas9 cutting efficiency or Cpf1 cutting efficiency or Cas12b cutting efficiency. It is further preferred that gRNA sequences suitable for inserting a tag sequence in the selected introns in the genome of the cell are identified according to their occurrence in the genome of the cell, preferably wherein the occurrence is 1. gRNAs of the present invention may also be single gRNAs (sgRNAs).

Cells to be intron-tagged are not limited and may be selected, inter alia, from the group consisting of HAP1 cells, U-2 OS cells, K562 cells, Hela cells, KBM7 cells, BT474 cells, MG-63 cells, SKNAS cells, A427 cells, A375 cells, A498 cells, RCH-ACV cells, HEK293T cells, A673 cells, SK-N-MC cells, A549 cells, SKMES1 cells, NCIH727 cells, THP1 cells, NB4 cells, MOLM13 cells, KASUMI-1 cells, HEL cells, NB-4 cells, HL-60 cells, RS4-11 cells, MOLT7 cells, aTC1 cells, bTC3 cells and Min6 cells. The person skilled in the art may also employ other cells/cell lines like, e.g., without being limiting, an adherent and/or a non-migratory cell line.

In one embodiment of means and methods of the present invention, the intron-tagged pool of cells comprises intron and/or exon tagged cells.

The present invention provides for novel intron-tagged pool of cells that can be obtained by the means and methods provided herein.

Accordingly, the invention further relates to a pool of cells, in particular an intron-tagged pool of cells, comprising a plurality of intron-tagged cells, wherein an individual cell, preferably each individual cell, in said pool of cells is characterized by at least two different tags in at least two different introns of at least two different genes.

An individual cell, preferably each individual cell, in said pool of cells may comprise at least two different tags in at least two different intron frames of at least two different genes. Preferably, an individual cell, preferably each individual cell, in said pool of cells may comprise two different tags in two different intron frames of two different genes, or three different tags in three different intron frames of three different genes. In other words, an individual cell, preferably each individual cell, in said pool of cells may preferably comprise (i) two different tags in two introns of two different genes, wherein the two introns have different intron frames, or (ii) three different tags in three introns of three different genes, wherein each of the three introns has a different intron frame.

Furthermore, said pool of cells and/or said plurality of intron-tagged cells may comprise at least 10, 100, 1000, 10000 or 20000 tagged introns, in particular, tagged introns of different genes; wherein preferably two or three introns are tagged per cell.

In particular, a gene, preferably each gene, comprising a tagged intron may be translated/expressed into a corresponding fusion protein. In particular, such a fusion protein may comprise at least part (or the entirety) of the amino acid sequence encoded by the tagged endogenous gene as well as the tag/label encoded by the corresponding tag sequence, as described herein.

Optionally, an individual cell, e.g. each individual cell, in said pool of cells may further comprise at least one tag in at least one exonic open reading frame, preferably in two or three different exonic open reading frames, preferably in two of three different genes.

The tags may be selected from fluorescent tags such as GFP, or tags suitable for detection by covalent or non-covalent binding to a detection reagent enabling detection by microscopy, e.g. by fluorescence or luminescence, such as a Halo tag or a Strep-tag, as described herein and in context of the present invention.

Furthermore, at least one additional cellular substructure may be labelled in at least one or all of the intron tagged cells, as described herein. Furthermore, at least one or all of the intron tagged cells and/or at least one cellular substructure thereof may be labelled with at least one further fluorescent and/or luminescent marker selected from the group consisting of miRFP670, mAmetrine, (mTag)BFP(2), fluorescently labeled antibodies, DAPI, Hoechst 33258, Hoechst 33342 and Hoechst 34580, as described herein. The inventive pool of cells provided herein and described, e.g., above, may be obtainable by or obtained by the inventive method for obtaining an intron-tagged pool of cells representing and/or comprising tagged introns, as described herein.

Furthermore, the novel intron-tagged pool of cells described herein may be, inter alia, characterized by comprising at least two different tags in at least two different intron frames/phases of at least two different genes per intron-tagged cell within said pool, as described herein.

As such the intron-tagged pool of cells of the present invention provides a novel and inventive tool for whole proteome analysis as well as for a valuable tool for drug screenings on a cellular basis which can be assisted by computer assisted/automatic means. In a further embodiment, the present invention also comprises a kit comprising said novel and inventive intron-tagged pool of cells. Such a kit is also particularly useful as means for drug screenings, drug evaluations, treatment monitoring, as research tool for basic sciences. Further uses of the inventive intron-tagged pool of cells are within the capabilities of the skilled artisan and are also illustrated herein.

In another embodiment of the present invention the identity of the tagged intron(s) comprised in the genome of an intron-tagged cell within an intron-tagged pool of cells may be analyzed and/or recognized. Said analysis or recognition may be automated recognition. This may be achieved by a sequence of steps: first, intron-tagged cells comprising genomically tagged introns are identified by obtaining (a) single-cell microscopy image(s) of the cell, said image(s) capturing (detectable) fluorescent and/or luminescent signal(s) emitted from (i) the expressed tag sequence(s) of the genomically tagged introns and/or (ii) labeled cellular substructure(s) and/or organelle(s). Next, integrated gRNA sequences or parts thereof, integrated into the genome of the intron-tagged cells are identified by sequencing. Next, model(s) of a computer vision algorithm are trained based on features from image(s) and gRNA sequencing data obtained and the identity of the tagged introns comprised in the intron-tagged cells within the intron-tagged pool of cells is automatically recognized. Accordingly, the invention also provides, in one embodiment for a method for automated recognition and/or computer-assisted recognition (or analysis) of the identity of the tagged intron(s) comprised in the genome of the intron-tagged cell(s) within an intron-tagged pool of cells as provided herein and/or as obtained by the means and methods of the present invention. In particular, said method is a computer-implemented method. Said method for automated recognition/computer-assisted recognition of the identity of the tagged intron(s) comprised in the genome of the intron-tagged cell may comprise the steps of:
(a) identifying said intron-tagged cells comprising genomically tagged introns by obtaining (a) single-cell microscopy image(s) of the cell, said image(s) capturing (detectable) fluorescent and/or luminescent signal(s) emitted from
   (i) the expressed tag sequence(s) of the genomically tagged introns and/or
   (ii) labeled cellular substructure(s) and/or organelle(s);
(b) sequencing the gRNA sequences, or parts thereof, integrated into the genome of the intron-tagged cells and as identified in step (a);
(c) training model(s) of a computer vision algorithm based on features from image(s) and gRNA sequencing data obtained in (a) and (b); and
(d) automatically recognizing the identity of the tagged introns comprised in the intron-tagged cells within the intron-tagged pool of cells.

As described herein above for the method for obtaining an intron-tagged pool of cells representing and/or comprising tagged introns, said method for automated recognition (or analysis) and/or computer-assisted recognition (or analysis) of the identity of the tagged intron(s) comprised in the genome of the intron-tagged cell within an intron-tagged pool of cells as provided herein, may also and additionally comprise the recognition/analysis of cellular structures or substructures. Again, such structures, substructure(s) and/or organelle(s) may be selected from the group consisting of cytoskeleton, cell membrane, cell wall, chloroplast, endoplasmic reticulum, Golgi apparatus, mitochondrion, and nucleus, and may be labeled. It is preferred that these cellular structures, substructure(s) and/or organelle(s) are labeled. Labels are known to the person skilled in the art and they may comprise fluorescent and/or luminescent marker(s) like, but not limited to, miRFP670, mAmetrine, (mTag)BFP(2), fluorescently labeled antibodies, DAPI, Hoechst dyes (i.e. Hoechst 33258, Hoechst 33342 and Hoechst 34580).

In order to implement the method for automated recognition/computer-assisted recognition of the identity of the tagged intron(s) comprised in the genome of the intron-tagged cell as provided herein, (a) computer algorithm(s), like computer vision algorithm may be employed. This may be based on machine learning. The person skilled in the art is readily in a position to employ such computer (vision) algorithm and is aware of corresponding model(s) for, e.g. automated analysis, like automated clone analysis. Such model(s), e.g. for automated clone recognition, may be based on random forests, support vector machines, variational autoencoders, recurrent neural networks (RNN), restricted Boltzmann machines, convolutional neural networks (CNN), etc.

In context of the method for automated recognition (or analysis) and/or computer-assisted recognition (or analysis) of the identity of the tagged intron(s) comprised in the genome of the intron-tagged cell within an intron-tagged pool of cells as provided herein, the features whereon the training model(s) in step (c) are based may comprise (i) the texture and granularity of cells, the (temporal) presence, absence, intensity, (subcellular) distribution and (co-) localization of fluorescent and/or luminescent signals and (ii) the identity of the tagged introns per cell. It is evident for the skilled artisan that the features under (i) are non-limiting and also other features may be employed.

In step (d) of the herein provided method for automated recognition (or analysis) and/or computer-assisted recognition (or analysis) of the identity of the tagged intron(s) comprised in the genome of the intron-tagged cell within an intron-tagged pool of cells, the identity of the tagged introns of the intron-tagged cells may be recognized/analyzed (preferably recognized) by the computer algorithm (like the computer vision algorithm), for example, with at least 70%, 80%, 90% or 95% accuracy, preferably with 98% accuracy.

As detailed above, the sequencing step in (b) allows the association of individual cells with tagged proteins by sequencing the individual gRNA. This may be achieved by sequencing the gRNA insert while it is not necessary to sequence the protein directly. This can either be done on whole population level or based on expressed proteins, for example subsequent to a cell sorting step based on the expressed tag. Accordingly, in the methods of the present invention, the gRNA insert, or a part thereof, of (a) cell(s) of the population is sequenced in the genome of said cell(s) or in the transcriptome of said cell(s). This sequencing step is in particular useful in order to provide additional information and/or to train the automated recognition (or analysis) or the corresponding model(s) for, e.g. automated analysis, like automated clone analysis.

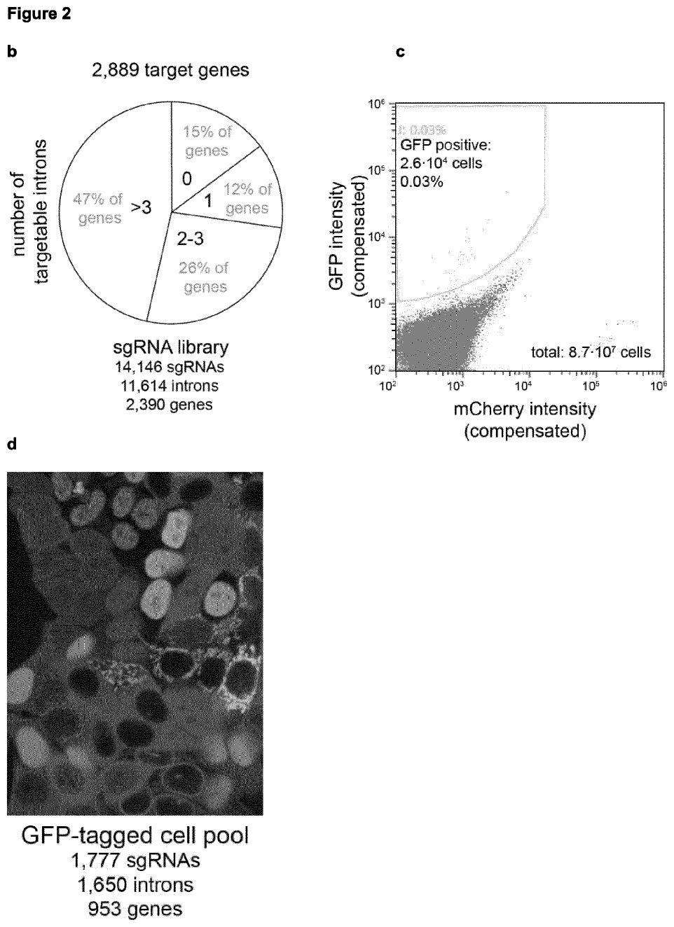

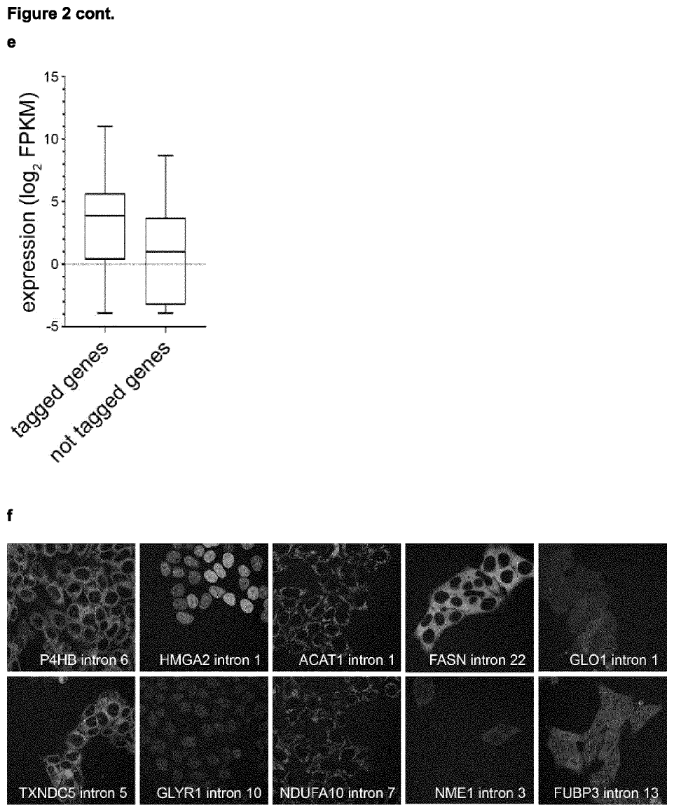

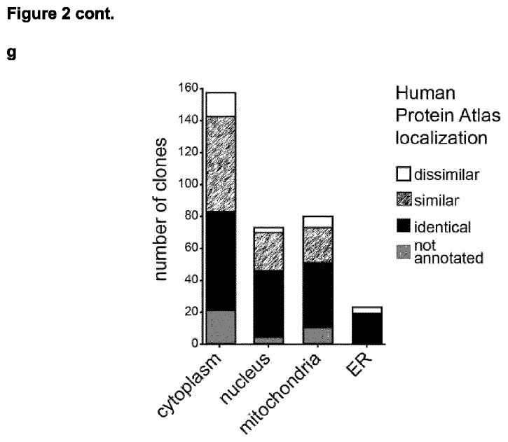

Sequencing of the gRNA insert in the transcriptome preferably further comprises a step of reverse transcription and the use of a sequencing vector as transduction vector. An exemplary vector suitable for sequencing is a CROP-Seq vector. In an exemplary embodiment, the procedure may be as in FIG. 2c. The method may be further adapted by using in situ sequencing using the Illumina NextSeq 500/550 kit v2, which provides a two-color system compatible with the “tag”/label” expressing cells.

As described herein and as shown in the appended examples, the introns to be targeted are selected based on the reading frame of the upstream exonic sequence, wherein the to be inserted sequence is in-frame with the exonic sequence. As shown in FIG. 2b, designing the illustrative intron targeting library comprised analyzing the target gene set regarding the number of available introns (FIG. 2b) and whether the introns were in the right reading frame (only one out of 3 reading frames is suitable to generate functional GFP integrations in context of that illustrative Example). Based on these criteria 14,146 sgRNA sequences for 11,614 introns in 2,390 genes were generated. Furthermore, the introns to be targeted can be comprised in genomic sequences of metabolic enzymes, chromatin proteins, kinases, genes coding for proteins in the ubiquitin/proteasome pathways, transcription factors, ion channels, transporters, receptors or at least one intron per protein coding gene in the genome can be targeted.

In another embodiment of the present invention, the effect of a perturbation on the proteome and/or gene expression levels of an intron-tagged cell within an intron-tagged pool of cells can be assessed by an exemplary but non-limiting sequence of the following steps: (a) selecting introns to be targeted in the genome of a cell; (b) identifying guide RNA (gRNA) sequences suitable for inserting a tag sequence in the selected introns in the genome of a cell to be intron-tagged; (c) cloning identified gRNA sequences and tag sequence into transfection and/or transduction vectors; (d) contacting a population of the cells to be intron-tagged with said transfection and/or transduction vectors of (c); (e) selecting of intron-tagged cells based on the presence of the tag sequence; (f) repeatedly performing steps (b) to (e) using a unique combination of the gRNA sequences and the tag sequence for each round of repetition, wherein said steps are performed 2 times, at least 3 times, at least 4 times, at least 5 times, at least 6 times and wherein (a) further fluorescent and/or luminescent marker(s) which may, inter alia, be selected from the group consisting of miRFP670, mAmetrine, (mTag)BFP(2), fluorescently labeled antibodies, DAPI, Hoechst dyes is/are used after the final round of repetition to label cellular substructure(s) and/or organelle(s) of the cell comprising the tagged introns; (f) identifying cells comprising genomically tagged introns by obtaining (a) single-cell microscopy image(s) of the intron-tagged cells, said image(s) capturing (detectable) fluorescent and/or luminescent signal(s) emitted from (i) the expressed tag sequence(s) of the genomically tagged introns and/or (ii) labeled cellular substructure(s) and/or organelle(s); (g) automatically recognizing the identity of the tagged introns comprised in the intron-tagged cells within the intron-tagged pool of cells; (h) exposing the intron-tagged cells within the intron-tagged pool of cells to a perturbation; (i) obtaining single-cell resolved time-course microscopy images of the intron-tagged cells within the intron-tagged pool of cells, said images capturing (detectable) fluorescent and/or luminescent signal(s) emitted from (i) the expressed tag sequence(s) of the genomically tagged introns and/or (ii) labeled cellular substructure(s) and/or organelle(s); and (j) assessing the effect of the perturbation based on single-cell analysis of the expressed tag sequence(s) of the genomically tagged introns and the labeled cellular substructure(s) and/or organelle(s) prior and after said perturbation. Accordingly, the present invention also provides for a method for assessing the effect of a perturbation on the proteome and/or gene expression levels of an intron-tagged cell within an intron-tagged pool of cells as provided by the means and methods of the present invention. This method for assessing the effect of a perturbation on the proteome and/or gene expression levels of an intron-tagged cell may comprise the steps of:
(a) selecting introns to be targeted in the genome of a cell;
(b) identifying guide RNA (gRNA) sequences suitable for inserting a tag sequence in the selected introns in the genome of a cell to be intron-tagged;
(c) cloning identified gRNA sequences and tag sequence into transfection and/or transduction vectors;
(d) contacting a population of the cells to be intron-tagged with said transfection and/or transduction vectors of (c);
(e) selecting of intron-tagged cells based on the presence of the tag sequence;
(f) repeatedly performing steps (b) to (e) using a unique combination of the gRNA sequences and the tag sequence for each round of repetition, wherein said steps are performed 2 times, at least 3 times, at least 4 times, at least 5 times, at least 6 times and wherein (a) further fluorescent and/or luminescent marker(s) (is/are used after the final round of repetition to label cellular substructure(s) and/or organelle(s) of the cell comprising the tagged introns;
(g) identifying cells comprising genomically tagged introns by obtaining (a) single-cell microscopy image(s) of the intron-tagged cells, said image(s) capturing (detectable) fluorescent and/or luminescent signal(s) emitted from
   (i) the expressed tag sequence(s) of the genomically tagged introns and/or
   (ii) labeled cellular substructure(s) and/or organelle(s);
(h) automatically recognizing the identity of the tagged introns comprised in the intron-tagged cells within the intron-tagged pool of cells;
(i) exposing the intron-tagged cells within the intron-tagged pool of cells to a perturbation;
(j) obtaining single-cell resolved time-course microscopy images of the intron-tagged cells within the intron-tagged pool of cells, said images capturing (detectable) fluorescent and/or luminescent signal(s) emitted from
   (i) the expressed tag sequence(s) of the genomically tagged introns and/or
   (ii) labeled cellular substructure(s) and/or organelle(s); and
(k) assessing the effect of the perturbation based on single-cell analysis of the expressed tag sequence(s) of the genomically tagged introns and the labeled cellular substructure(s) and/or organelle(s) prior and after said perturbation.

It is also possible that said steps (a) to (f) are omitted and said steps (g) to (k) of the above method for assessing the effect of a perturbation on the proteome and/or gene expression levels of an intron-tagged cell are directly performed on the inventive pool of cells provided herein which may be obtainable by performing the above steps (a) to (f). Furthermore, it is also possible that said steps (g) and (h) are omitted instead of in addition of said steps (a) to (f), and the identity of the tagged introns comprised in the intron-tagged cells within the intron-tagged pool of cells is determined by another method, e.g. by single cell in situ sequencing as described herein. In particular, said above steps (i) to (k) may be also directly performed on the inventive pool of cells provided herein. Advantageously, the inventive method for obtaining an intron-tagged pool of cells and the inventive pool of cells according to the present invention allow for a further miniaturization of compound/drug screening platforms and increase the efficiency of methods for assessing the effect of a perturbation on the proteome and/or gene expression levels and/or drug screening methods. That is, inter alia, because multiple, e.g. 2, 3, 4, 5 or 6, intron or exon tagged genes and corresponding fusion proteins may be assayed per individual cell in the pool of cells, and there may be only little overlap between the tagged genes of the different clones of cells in the pool.

The perturbation to be assessed by the inventive method for assessing the effect of a perturbation on the proteome and/or gene expression levels of an intron-tagged cell provided herein may be selected from radiation, an inorganic chemical compound, an organic chemical compound, a biological compound, temperature, nutrient depletion, ion concentration(s). This method is particularly useful in drug screenings and drug evaluations. Accordingly, said “perturbation” to be assessed or analyzed may also be caused by a (potential) drug to be used in medical intervention. Accordingly, the means and methods provided herein are particularly useful for testing drugs. The drugs to be assessed/tested may be used in the treatment/medical intervention of, e.g., cancerous diseases and/or neurological diseases.

The embodiments provided herein above for the inventive method for obtaining an intron-tagged pool of cells representing and/or comprising tagged introns and/or the inventive method for automated recognition of the identity of the tagged intron(s) comprised in the genome of an intron-tagged cell within an intron-tagged pool of cells also apply, mutatis mutandis, for the herein provided method for assessing the effect of a perturbation on the proteome and/or gene expression levels of an intron-tagged cell within an intron-tagged pool of cells.

The analysis step of the means and methods provided herein may be carried out in form of a single-cell analysis. Accordingly, and in certain embodiments of the present invention, the single-cell analysis of the expressed tag sequence(s) of the genomically tagged introns may be based on the alteration of (temporal) presence, absence, amount, (subcellular) distribution and (co-) localization of said expressed tag sequence(s) of the intron-tagged cells within the intron-tagged pool of cells.

As disclosed herein, the present invention provides improved methods for monitoring the effect of an environmental factor on the proteome or parts thereof of a cell as provided in WO 2021/099273 (incorporated by references). The invention as already provided in WO 2021/099273 relates to method for monitoring the effect of an environmental factor on the proteome or parts thereof of a cell, the method comprising the steps of:
(a) selecting introns to be targeted in the genome of a cell;
(b) identifying guide RNA (gRNA) sequences suitable for inserting a tag in the selected introns in the genome of the cell;
(c) cloning identified gRNA sequences and tag sequence into transduction vectors;
(d) contacting a population of the cell with said vectors of (c) to integrate the tag of (b) into selected introns;
(e) exposing cell population to environmental factor; and
monitoring the effect of the environmental factor on the proteome based on the detection of the tag prior to exposure of the cell population to the environmental factor and subsequent to exposure of the cell population to the environmental factor.

Accordingly, the present invention further relates to a method for monitoring the effect of an environmental factor on the proteome or parts thereof of a cell comprising a step of exposing the inventive pool of cells provided herein comprising, in particular, intron- and/or exon-tagged cells as described herein, to an environmental factor; and monitoring the effect of the environmental factor on the proteome based on the detection of the tags prior to exposure of the cell population to the environmental factor and subsequent to exposure of the cell population to the environmental factor.

In context of this invention (as in WO 2021/099273), the term “part(s) of the proteome” relates to a substantial part of the proteome, i.e. at least 100, at least 200 at least 300 at least 400 at least 500, at least 600, at least 700 and more preferably at least 900 expressed genes (coding for proteins).

In particular and in contrast to the prior art, in particular Serebrenik et al., the invention provided in WO 2021/099273 allows scalability to enable pooled protein tagging of a multitude of metabolic enzymes and epigenetic modifiers. As shown in the appended Examples, more than 900 metabolic enzymes were targeted. Exposing the GFP-tagged cells to compounds to monitor drug effects on the localization and levels of hundreds of proteins in real time in a pooled format, followed by identification of responding clones by in situ sequencing of the expressed intron-targeting sgRNA that corresponds to the tagged protein, as shown in FIG. 2a, represents a major advantage over methods of the prior art. Again, the means and methods provided in this invention is even improved over the methods as provided in WO 2021/099273.

The following embodiments are not only comprised in WO 2021/099273 but are also to be employed in context of this invention and the means and methods provided herein.

The design of(s) gRNA sequences may be based on cutting efficiency. Thus, the gRNA sequences suitable for inserting a tag in the selected introns in the genome of the cell may be identified according to Cas9 cutting efficiency or Cpf1 cutting efficiency or Cas12b cutting efficiency. Additionally, or alternatively, the gRNA sequences suitable for inserting a tag in the selected introns in the genome of the cell are identified according to their occurrence in the genome of the cell, preferably wherein the occurrence is 1.

The vector further encodes for a tag that is to be inserted into the intron. The tag can be any tag allowing detection subsequent to integration or expression. Preferably, the tag is a fluorescence tag (preferably green fluorescent protein (GFP or enhanced GFP) or yellow fluorescent protein (YFP), or red fluorescent protein (RFP) or a tag suitable for detection by covalent (e.g. Halo tag, Clip tag, Snap tag) or non-covalent (e.g. Strep-tag, HA tag, dTag) binding to a detection reagent enabling detection by microscopy by fluorescence or luminescence.

Once the sgRNA sequences and the tag sequences have been selected and cloned, the library of sgRNA vectors is contacted with a population of a cell to integrate the tag of into selected introns.

In order to select for infected cells, a selection marker can be comprised in the vector. An exemplary marker is the puromycin selection marker also present on the vector, e.g. the CROP-seq vector.

In an exemplary method, transient transfection can subsequently be used to introduce a plasmid for expression of Cas9 (that would introduce a cut specifically in an intron as specified by the sgRNA/gRNA previously introduced to the same cell with the CROP-seq vector) and a generic sgRNA/gRNA. Yet, Cas9 (or other suitable enzymes such as Cpf1 or Cas12b) can be also introduced into the cells in other ways or be already contained in the cells to be tagged from the beginning, as described herein.

A second plasmid can also be introduced that acts as a generic donor plasmid that provides the tag sequence, for example an EGFP sequence, to be integrated into the intron. This plasmid contains a Cas9 cut-site (targeted by a generic sgRNA sequence that may be present, e.g., on the Cas9 plasmid), a splice acceptor site, tag sequence (e.g. EGFP), a splice donor site and another Cas9-cut site (targeted by the generic sgRNA sequence that may be present, e.g., on the Cas9 plasmid). As an alternative to the generic donor plasmid with two Cas9 cut-sites, minicircle DNA that does not comprise a plasmid backbone but comprises a single Cas9 cut-site, a splice acceptor site, tag sequence (e.g. GFP or EGFP) and a splice donor site may be used. When using a minicircle, the intron tagging efficiency is increased, due to the lack of a plasmid backbone that can get integrated at the intronic integration site instead of the tag sequence containing fragment. The methods may further comprise selection for cells that are successfully transfected (e.g. blasticidin marker on the Cas9 plasmid) and expansion of the cells, for example over a period of 5 days.

The methods of the present invention as well as in WO 2021/099273 may further comprise a step of separating tagged cells from non-tagged cells. The separation method depends on the tag that is used. In a preferred embodiment, the cells are fluorescence tagged and the cells are separated using FACS. Accordingly, FACS or an alternative separation method can be used to sort out targeted cells. In addition, tagged proteins can be selected according to expression levels. That is, all proteins are expressed at endogenous levels, and some proteins are expressed to very low levels. To differentiate between expression levels, a further parameter may be used (e.g. a further channel during selection), e.g. for cell-specific background fluorescence and sort cells that are enriched for the tag, for example GFP or EGFP (FIG. 2c).

The sorted pool of tagged cells or the unsorted pool comprising tagged cells may further be characterized using a suitable method based on the introduced tag. For example, protein expression, protein localization, surface expression, protein-protein interaction, protein stability, and/or protein mobility may be monitored using a suitable detection method.

As such, the invention also relates to a population of cells comprising multiple cells each comprising an inserted tag sequence in an intron of said cell, in particular at least two tag sequences inserted into at least two different introns of said cell, wherein a tag is inserted in-frame with the preceding exonic sequence and wherein the intron(s) into which the tag(s) is/are inserted is/are (essentially) different between cells, i.e. cell clones.

As detailed above, the cells may also be characterized by sequencing. In one exemplary embodiment, the intron tagged cell pool can be characterized by PCR amplifying the integrated sgRNA sequence from genomic DNA, next generation sequencing and mapping back to the sequences in the designed sgRNA library. This way it was determined that more highly expressed genes were more likely to be successfully tagged (FIG. 2e).

In a further exemplary embodiment, the intron tagged cell pool can be further characterized by diluting to single cells and growing them up to large colonies on a 96-well plate. These single-cell derived clones can be characterized by imaging (FIG. 2f) to identify the targeted protein again by PCR amplifying the integrated sgRNA sequence from genomic DNA, next generation sequencing and mapping back to the sequences in the designed sgRNA/gRNA library. This way it could be confirmed that the vast majority of cells indeed harbor only a single tagged protein. Furthermore, it was observed that the vast majority of protein locations in the clones corresponded to those identified by antibody-based staining of the targeted protein in the Human Cell Atlas (FIG. 2g).

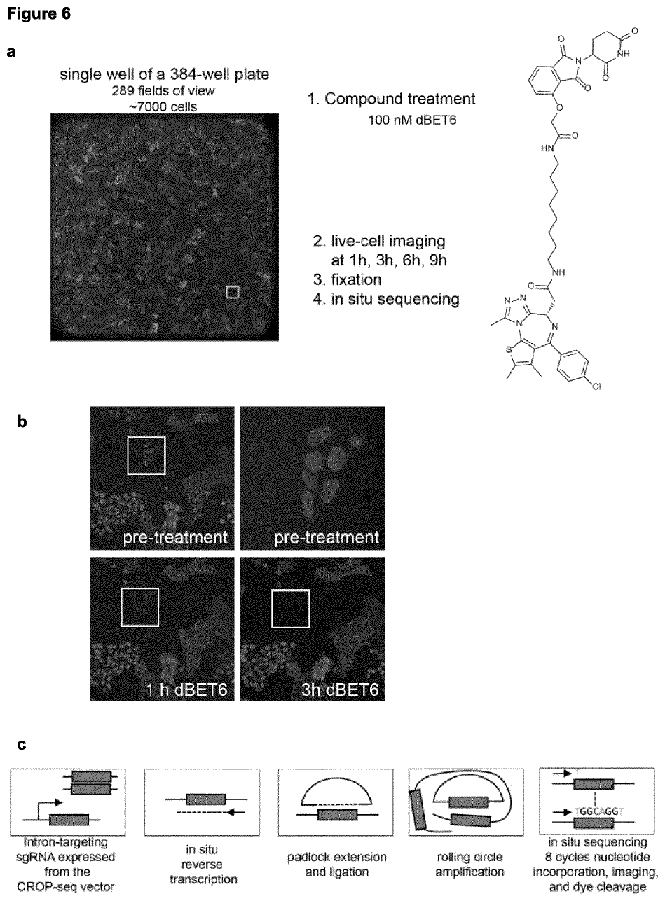

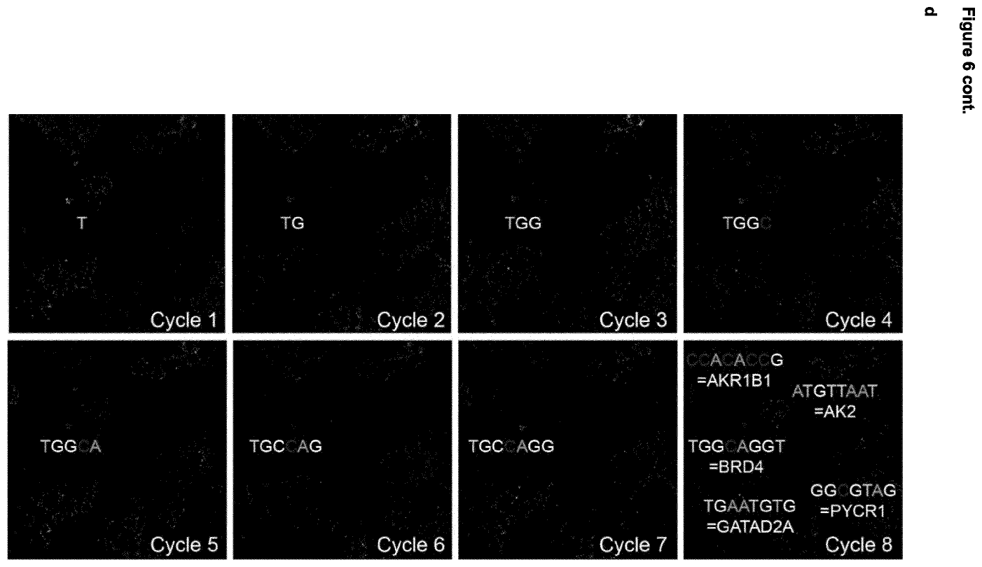

The environmental factor to be assessed in the means and methods provided herein may be, inter alia, selected from radiation, a chemical compound, a biological compound, temperature, nutrient depletion, ion concentrations or combinations thereof. In an exemplary embodiment, the method may comprise plating of cell pools at conditions of approximately 7,000 cells per well in a 384-well plate (FIG. 6a). While the method works for a single cell per tagged protein, it is preferred to have at least 5 cells per tagged protein for robustness. An image from the cell pool before compound treatment is taken. Subsequently, the cells are exposed to the environmental factor, for example by adding a chemical compound. The chemical compound may be a drug, a small molecule, an siRNA, an shRNA/sgRNA virus, a protein. In one embodiment, the environmental factor may be at different intensities/concentrations for different cell populations, for example to obtain dose-response curves. Subsequently, at least one further image is taken. However, multiple images may be taken in order to follow the effect on the time scale. For example, images may be taken after 1 h, 3 h, 6 h, 9 h, but this timeframe can be freely adjusted (FIG. 6b).

The invention is further illustrated by the following non-limiting figures and examples:

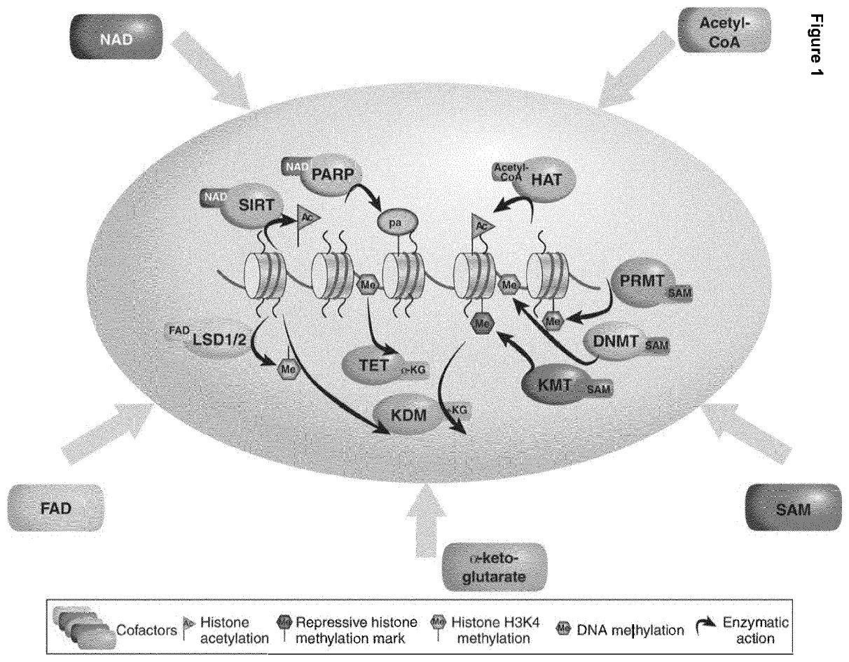

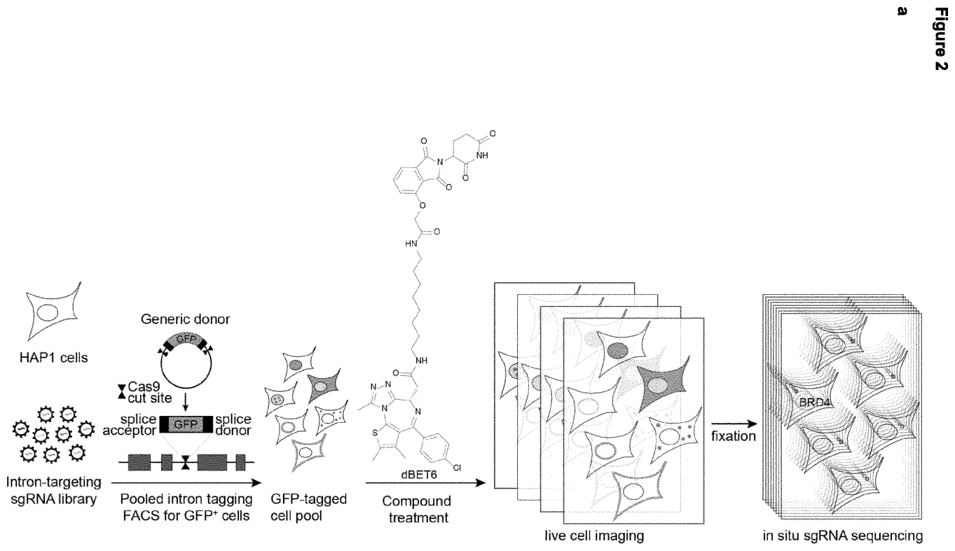

FIG. 1: Many cytoplasmic and mitochondrial metabolic enzymes are found in the nucleus. The subcellular localization of some metabolic enzymes changes during the cell cycle or in response to perturbations. Protein localization of can be studied by immunofluorescence staining or by endogenously tagging proteins on the N- or C-terminus. (Figure from Berger and Sassone-Corsi (2016), Cold Spring Harb Perspect Biol 8: a019463)

FIG. 2: Pooled GFP intron-tagging of metabolic enzymes. a. Schematic outline of the approach. b. Identification of targetable introns within metabolic genes. c. FACS sorting of clones with successful GFP-tagging by signal enrichment over background mCherry intensity. d. Representative image of sorted GFP-tagged cell pool. e. Comparison of RNA-seq expression in HAP1 cells between genes for which GFP-tagged cells could be isolated and genes that were targeted in the sgRNA library but did not result in successful clone isolation. f. Representative images of individual clones isolated by single cell dilution and identified by sgRNA NGS. g. Comparison of localizations of 335 individually isolated clones to localization annotations in the Human Protein Atlas.

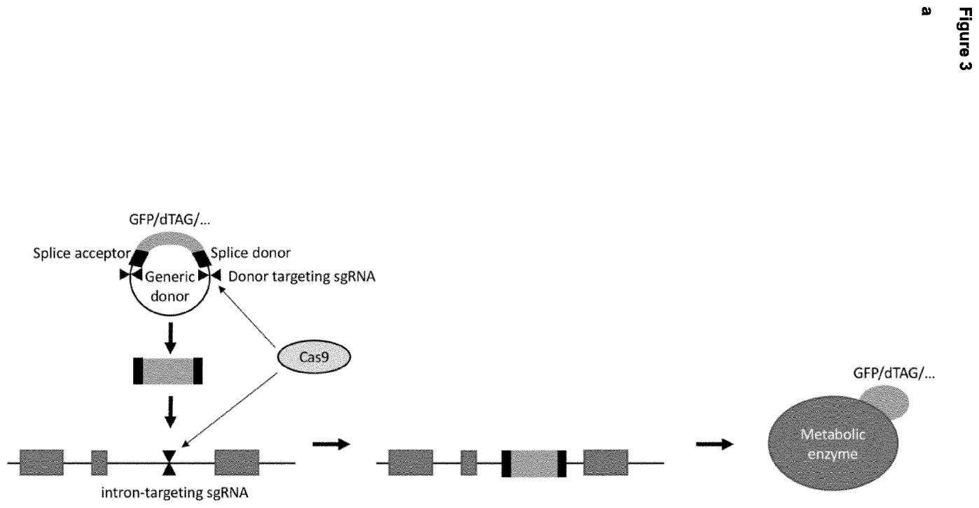

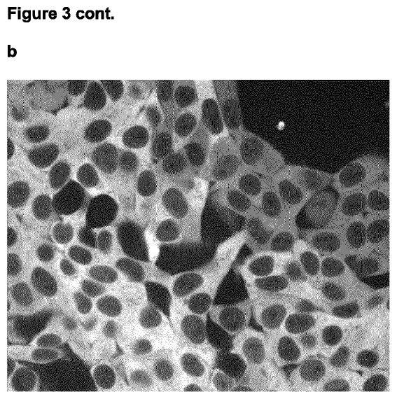

FIG. 3: Knock-in of a protein tag using an intron targeting strategy. a. Intron-tagging strategy with an intron targeting sgRNA, and a generic sgRNA targeting a donor plasmid targeting that provides a synthetic exon with the protein tag. This synthetic exon without homology arms is integrated into the intronic target site specified by the specific sgRNA via more NHEJ. This is a scalable targeting strategy as the same generic donor can be applied for all targets. b. Representative image confirming successful targeting of intron 26 in MTHFD1 with 2.2% efficiency.

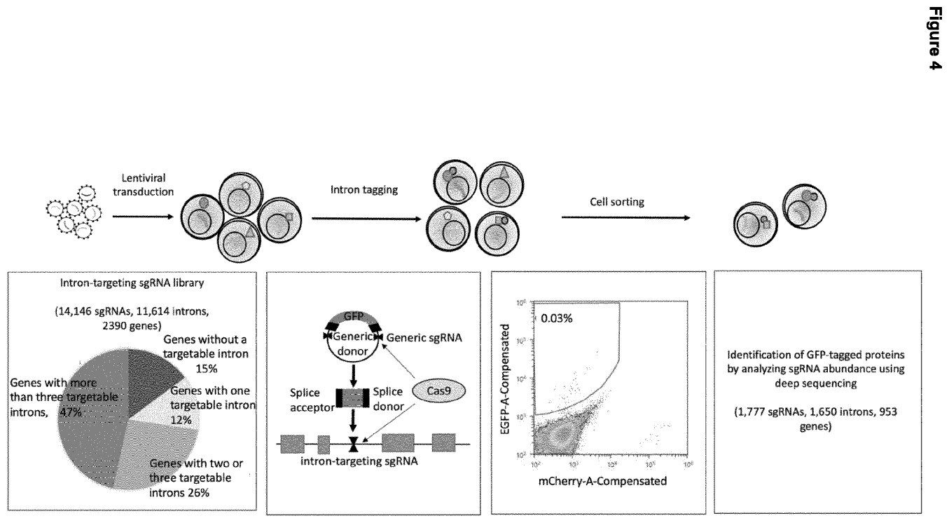

FIG. 4: Pooled protein-tagging of metabolic enzymes and epigenetic modifiers

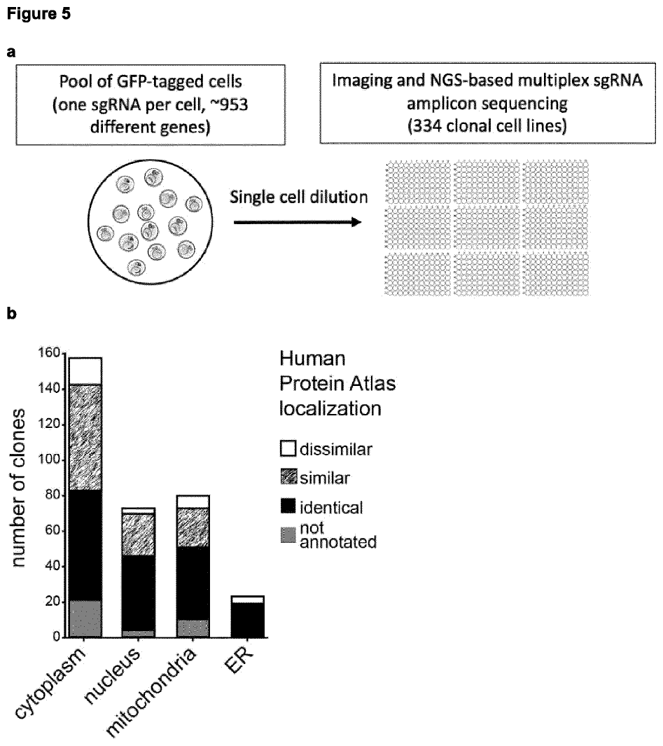

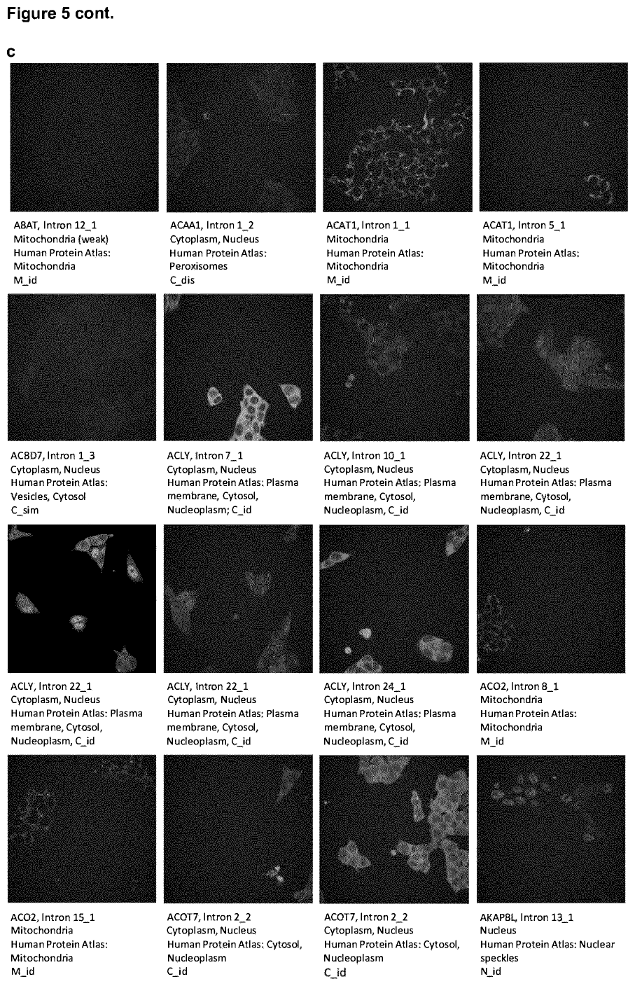

FIG. 5: Isolation of 334 clonal cell lines and comparison of subcellular protein localization to The Human Protein Atlas. a. Single cell cloning approach by single cell dilution to 96-well plates. b. Comparison of localizations of 335 individually isolated clones to localization annotations in the Human Protein Atlas. c. Representative images for the first 16 clones alphabetically

FIG. 6: Compound screening on cell pools followed by in situ sequencing enables the detection of protein-specific compound effects. a. Stitched image of 289 fields of view representing an entire well on a 384-well plate containing approximately 7,000 individual cells. b. Identification of a clone with rapid loss of GFP signal following treatment with 100 nM dBET6, while neighboring clones are unaffected. c. Outline of the in situ sequencing approach. d. Images from 8 cycles in situ sequencing of the area shown in panel b. e. Selected images for clones showing localization changes following dBET6 treatment.

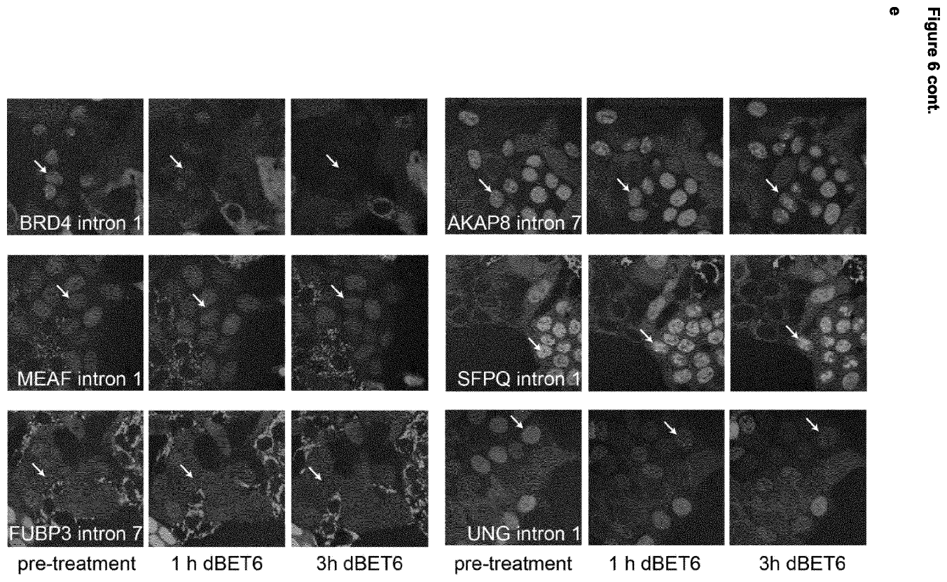

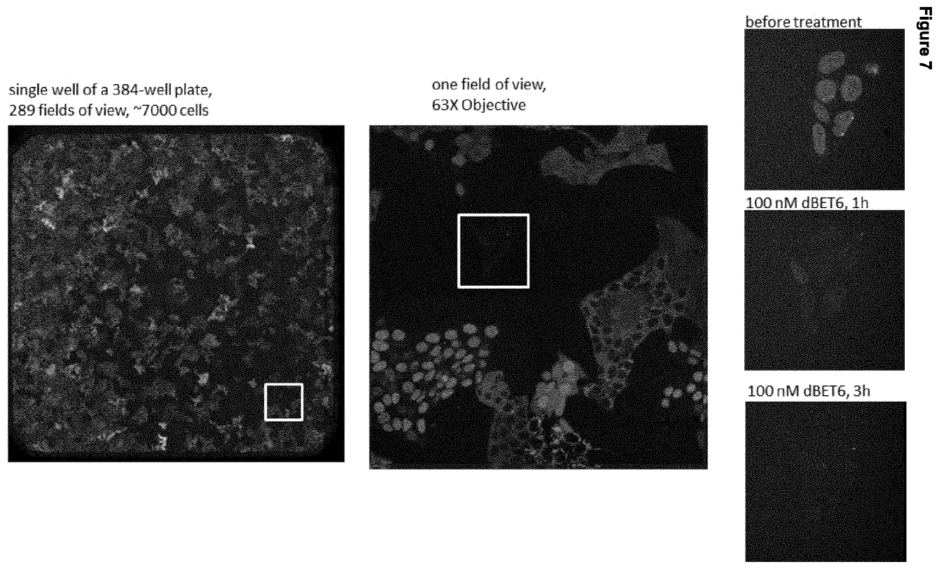

FIG. 7: Pooled screening approach to study drug effects on protein levels and localization.

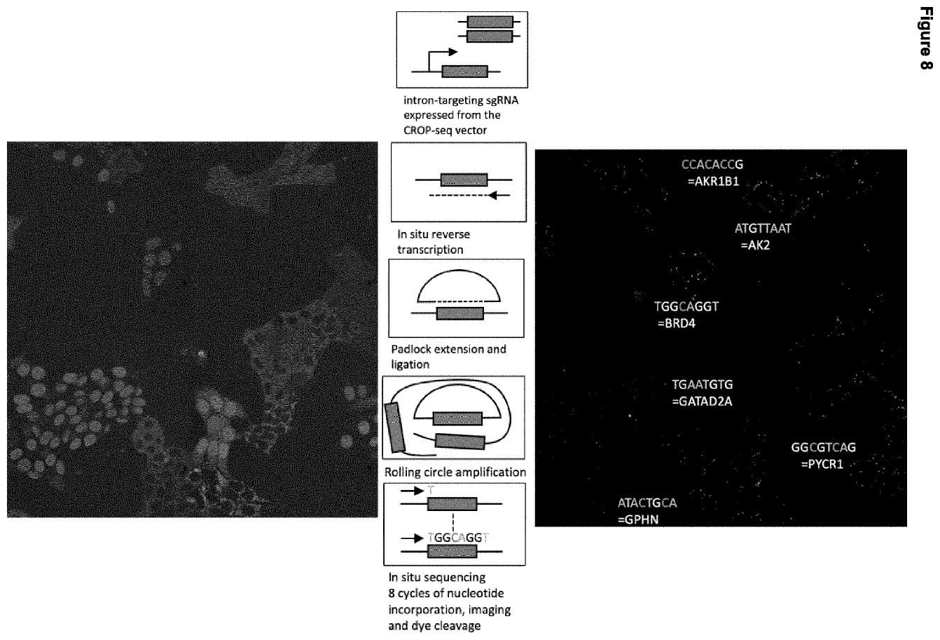

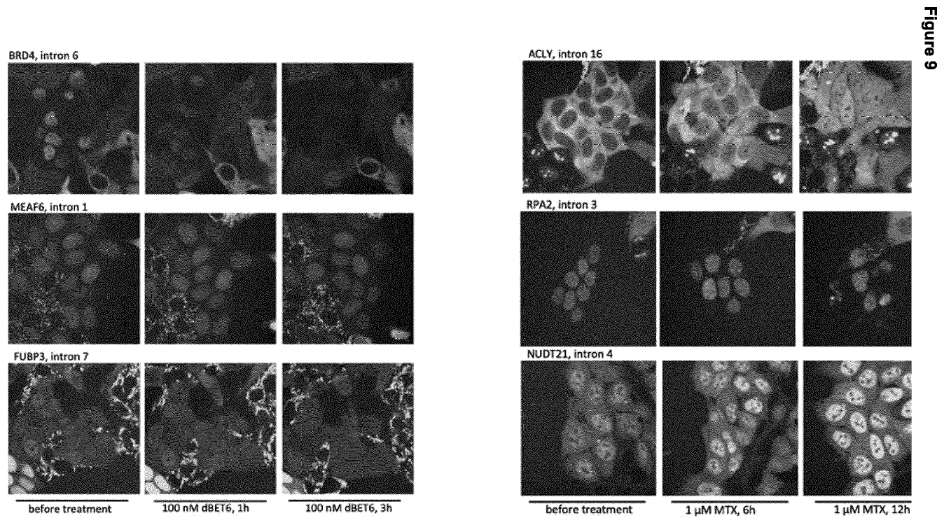

FIG. 8: In situ sequencing to identify the tagged protein in individual clones.

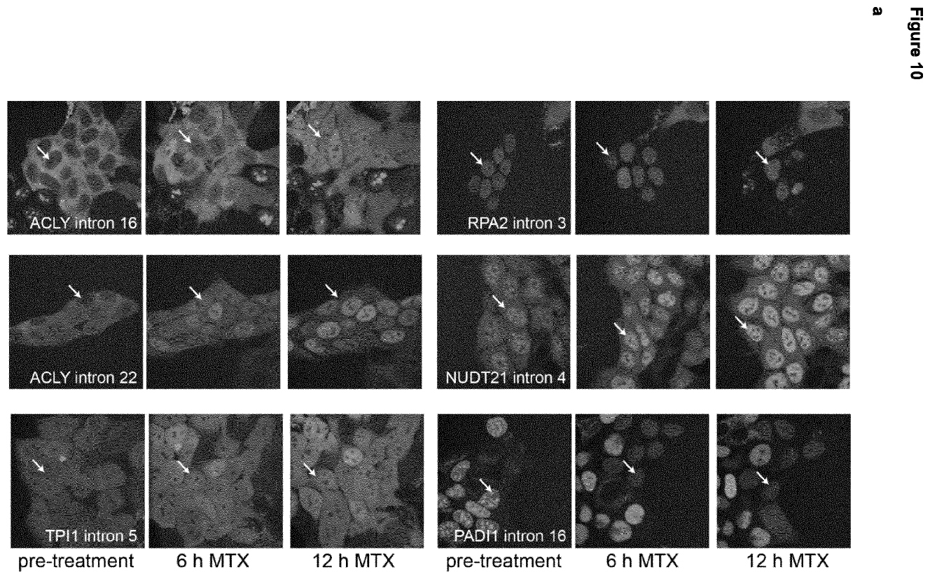

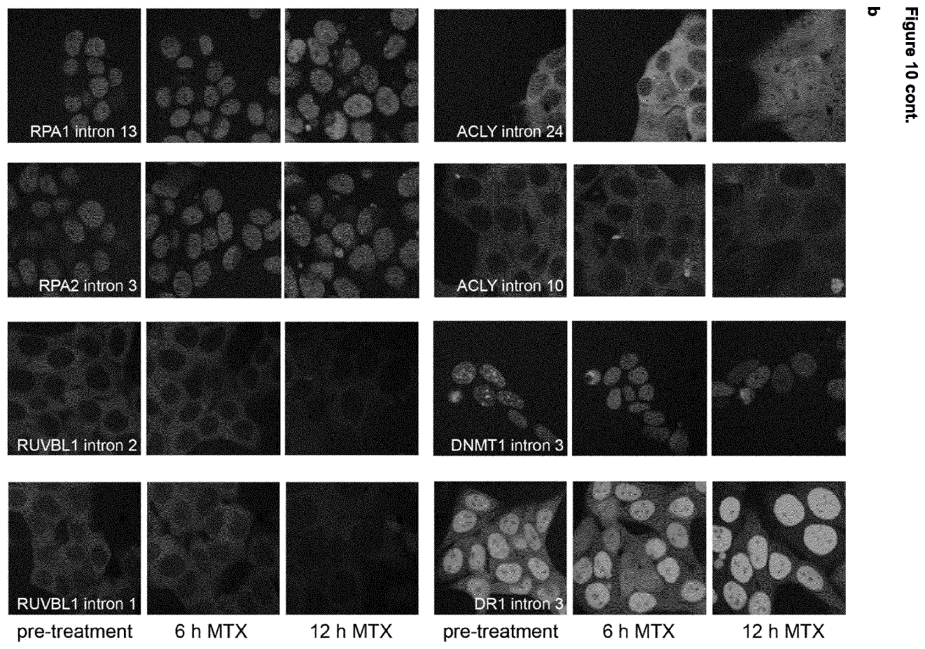

FIG. 9: Effects on protein localization and levels induced by the BRD4 degrader dBET6 and methotrexate.

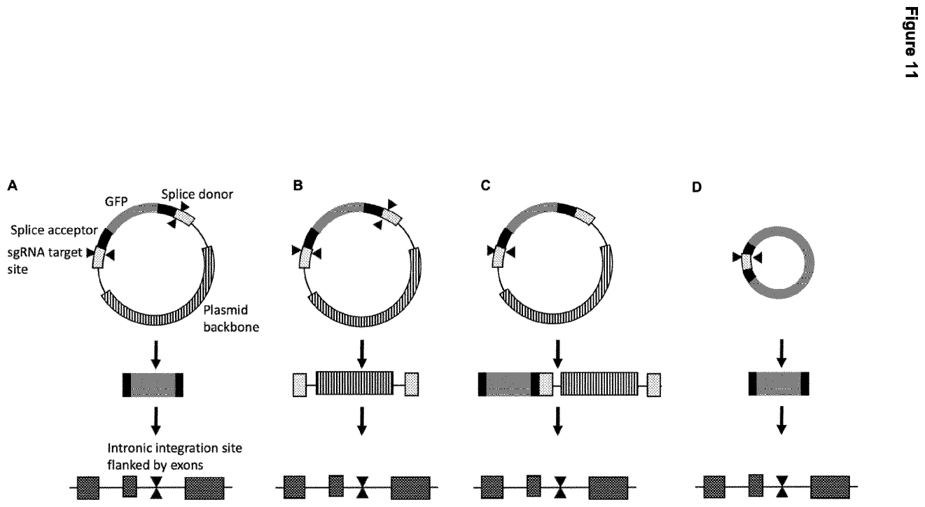

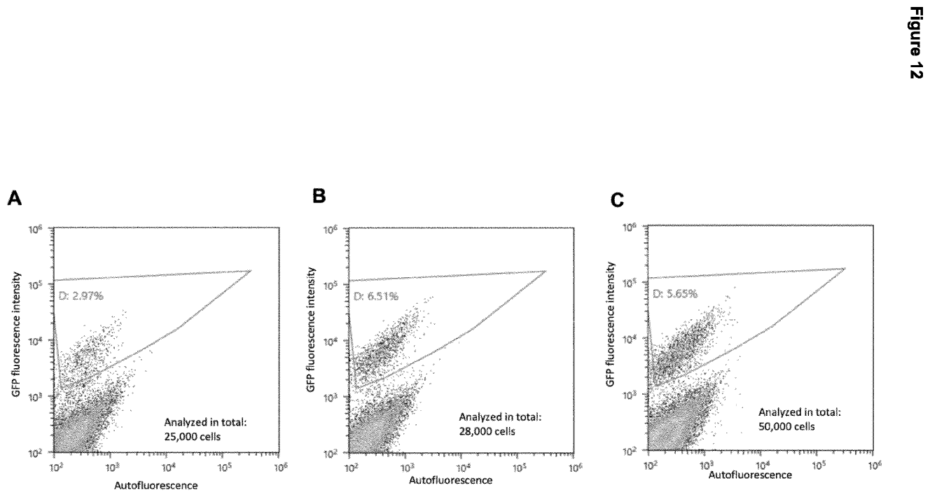

FIG. 10 Time-dependent changes in protein localization and levels following treatment with 1 □M methotrexate. a. Selected images showing changes following methotrexate treatment in cell pools, annotated by clone identification from in situ sequencing. b. Selected images showing changes in 335 arrayed clones exposed to methotrexate.

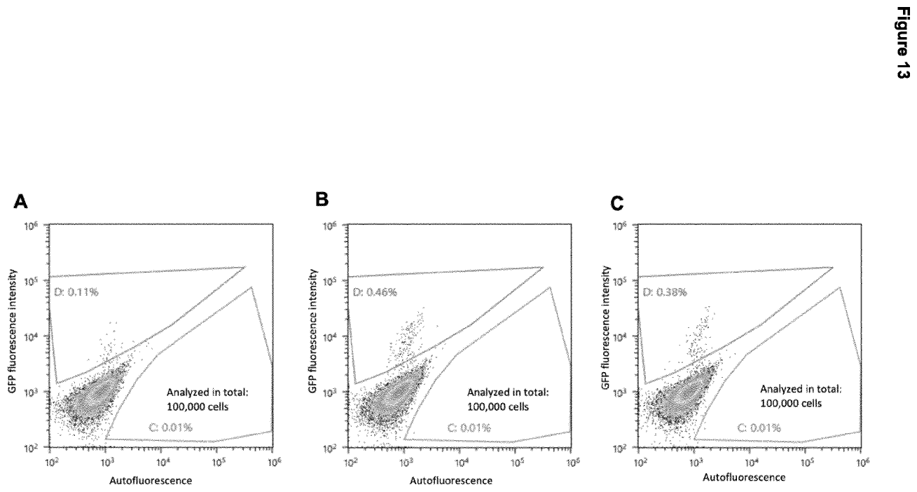

FIG. 11A. The GFP donor plasmid is cut at two sgRNA targeting sites followed by integration of a fragment containing GFP flanked by splice acceptor and splice donor sites. B. The plasmid backbone gets integrated instead of the GFP containing fragment. C. The GFP donor plasmid is cut only once and the entire plasmid gets integrated. D. A minicircle has only one sgRNA target site and no plasmid backbone can get integrated.

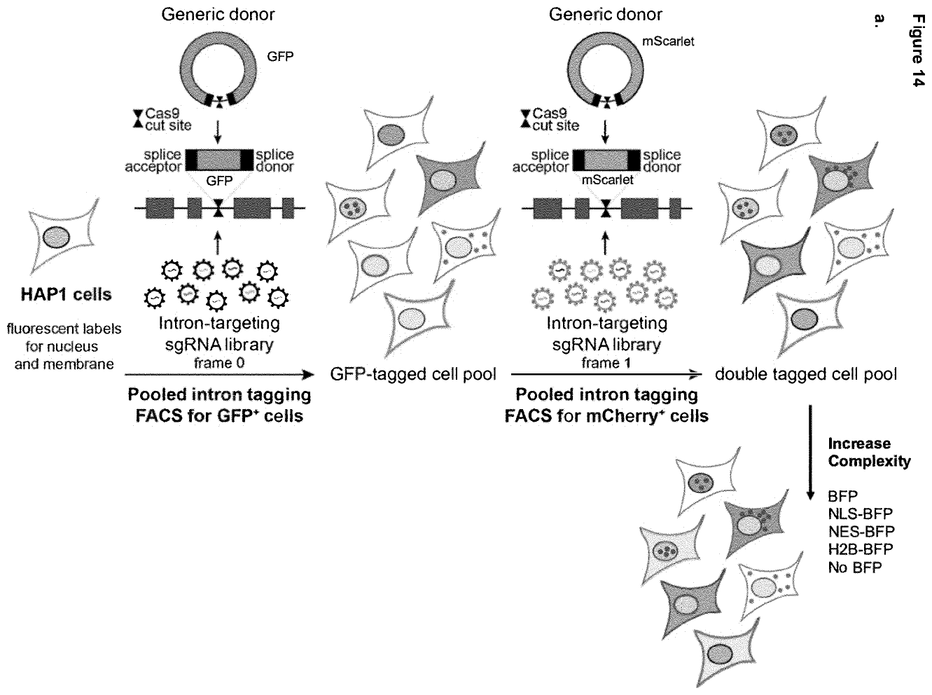

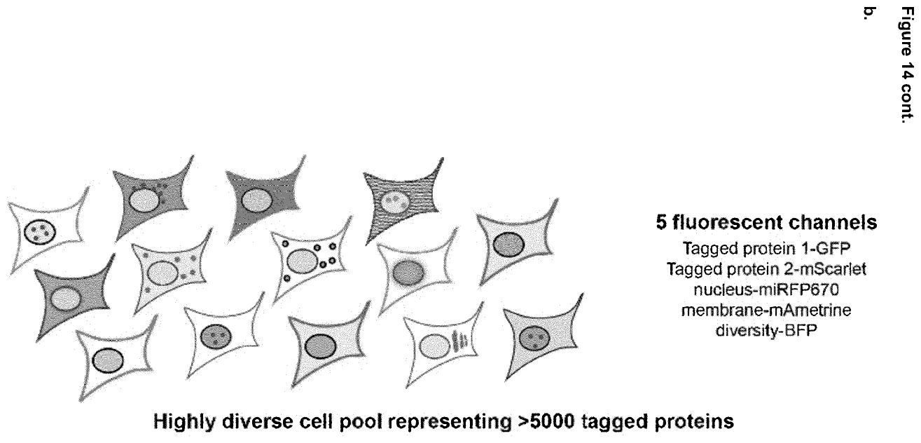

FIG. 12 Tagging rates when targeting the CANX gene and A) the conventional GFP donor plasmid, B) the same amount of minicircle DNA and C) one third the amount of minicircle DNA.

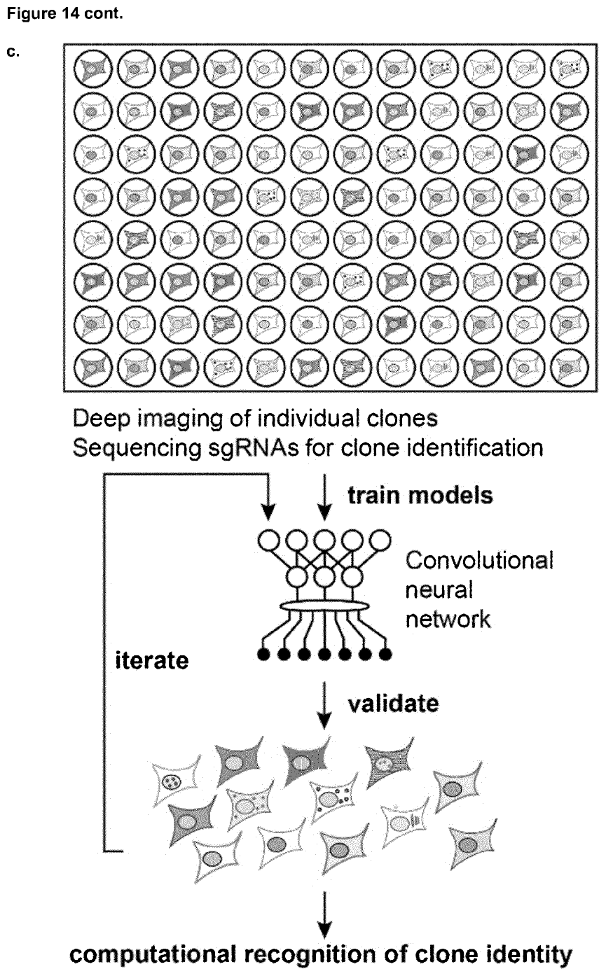

FIG. 13 Tagging rates when targeting the CANX gene and A) the conventional GFP donor plasmid, B) the same amount of minicircle DNA and C) one third the amount of minicircle DNA in cell populations that were not enriched for transfected cells.

FIG. 14 Workflow of generating a pool of cells comprising cells with multiple tagged introns and automated clone recognition thereof. a. A method for generation for a cell pool in which each cell harbors multiple proteins expressed from their endogenous promoter and tagged with fluorescent proteins of different colours, while at the same time enabling the unambiguous identification of the tagged protein based on an sgRNA sequence used to identify the tagging site. This strategy is implemented by using specific combinations of fluorescent tags and sgRNA libraries, that enable the specific association of each fluorescent tag with a specific sgRNA library. In particular, this can be achieved by using intron-targeting sgRNA libraries in the three different reading frames, and constructs for splice acceptor/donor flanked fluorescent proteins of different color in the different reading frames, possibly also in combination with exon-targeting sgRNA libraries in the three different reading frames in combination with constructs for fluorescent proteins of different color in the different reading frames. Thereby, this strategy enables tagging up to six different genes with different fluorescent tags, while still being able to uniquely assign them by sequencing the six different sgRNAs present in the cell. The genes to be tagged can be pre-selected by the design of the sgRNA libraries to cover any subset from 2 genes up to the entire proteome. In addition to the minimum of two tagged proteins expressed from their endogenous promoters, additional fluorescent channels can be used to either (i) include other proteins expressed from their endogenous promoters; or (ii) include fluorescent proteins that mark certain cellular structures (e.g. nucleus, membrane) to aid cell segmentation; or (iii) include fluorescent markers of randomized localization/intensity to enable diversification of the cell pool. b. A highly diverse cell pool representing >3000 tagged proteins. In this exemplary cell pool resulting from two rounds of intron tagging and additional stable integration and expression of fluorescent markers (GFP and mScarlet), in every cell two different genes are tagged, depending on the two intron-targeting sgRNAs transcribed by that cell and every cell expresses two additional structural markers to aid cell segmentation (miRFP670 and mAmetrine) and one fluorescent marker to diversify the pool (BFP). c. A method for automated recognition of the identity of the tagged proteins in each cell of the pool of cells, based on (a). On the single cell level, obtaining high resolution microscopy images in all fluorescent channels, and high quality sequencing data of all sgRNAs present in this single cells. This step needs to be only completed once for each cell pool, and can be implemented either by single-cell subcloning of the cell pool to individual wells, imaging of the resulting colonies, DNA isolation on a well-by-well basis, amplification of the integrated sgRNA sequence and DNA sequencing; or by high-quality in situ sequencing of the cell pool. Based on the individual images and the known identify of the cells, a computer vision algorithm is trained to discriminate the majority of all cells in the cell pool. This step needs to be completed only once for each cell pool. d. The application of the cell pool to identify and characterize drug candidates that change the abundance or localization of selected proteins in high throughput based on (a) imaging the cell pool prior to drug exposure; (b) exposing the cell pool to a perturbation e.g. with a drug candidate; (c) imaging the response of the cell pool to the exposure, possible in a time course; (d) using the automated clone recognition algorithm to identify the nature of the tagged proteins in each single cell of the cell pool from the images prior to drug exposure; (e) identify responding proteins by performing single-cell comparison of protein levels and localization prior and after drug treatment.

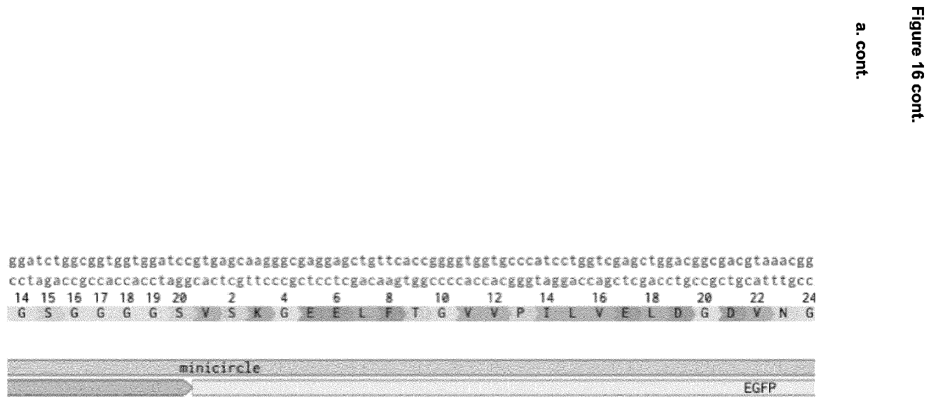

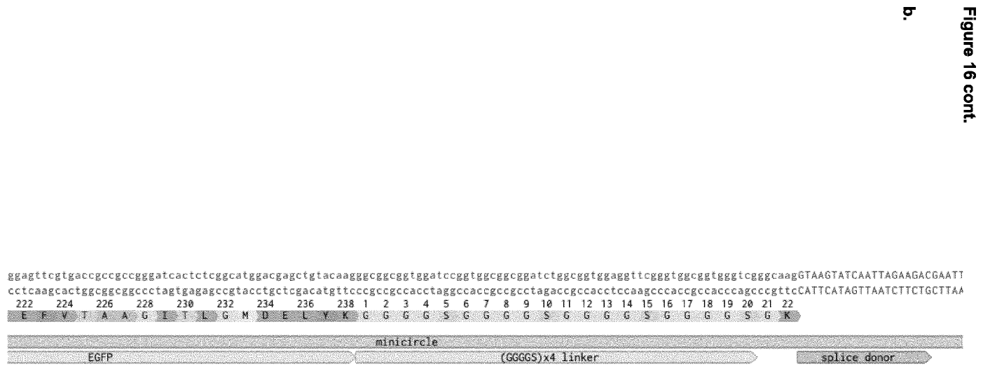

FIG. 15. Design of genome-wide sgRNA libraries targeting introns of different frames. a. In this exemplary workflow for designing such a library, the Ensembl genome browser (ensemble.org) was used to obtain transcript information, genomic coordinates of introns and intron frames (also called intron phases). Intron-targeting sgRNAs are then generated using computational tools that use genomic coordinates as an input such as GuideScan (guidescan.com). The generated sgRNAs are then ranked based on on- and off-target scores and only sgRNAs for certain frames are selected. b. The ratio of different intron frames or phases of all introns in the human genome. c. Size of two genome-wide libraries containing sgRNAs targeting different intron frames.

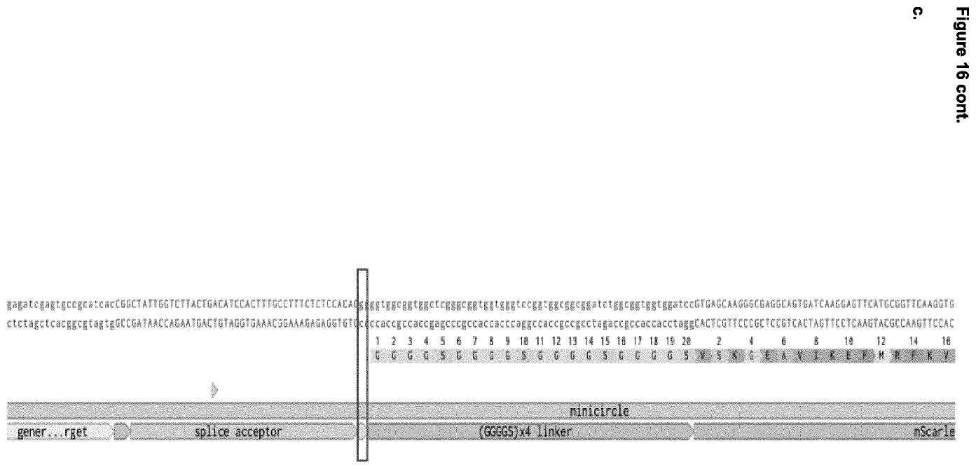

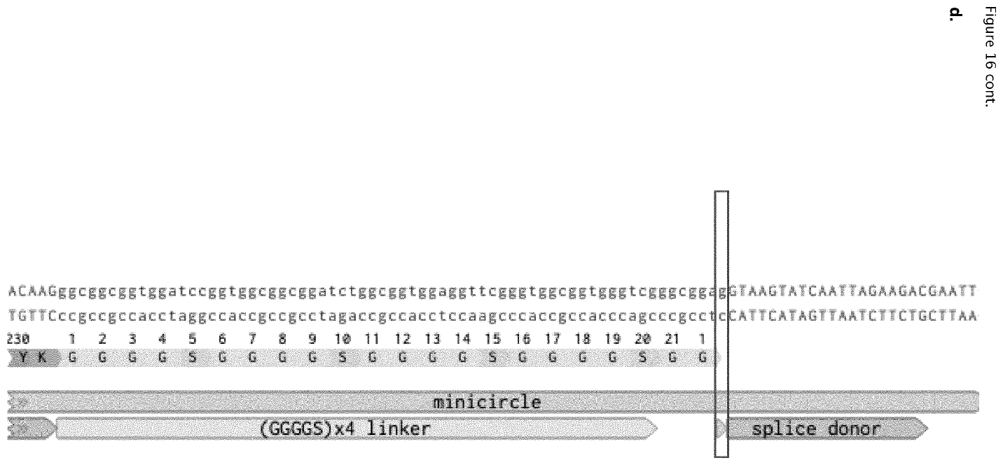

FIG. 16. Minicircle constructs for multiple tagging. a. EGFP minicircle to be used with sgRNAs targeting introns of frame 0. Shown is the part containing the generic sgRNA targeting site followed by a splice acceptor site, followed by the sequence coding for an amino-acid linker, followed by EGFP (only N-terminal part is shown). b. Shown is the part of the EGFP minicircle containing EGFP (only the C-terminal part is shown), followed by the sequence coding for an amino-acid linker, followed by a splice donor site. c. mScarlet minicircle to be used with sgRNAs targeting introns of frame 1. Shown is the part containing the generic sgRNA targeting site followed by a splice acceptor site, followed by two extra nucleotides that are necessary when targeting frame 1 introns (circled in red), followed by the sequence coding for an amino-acid linker, followed by mScarlet (only N-terminal part is shown). d. Shown is the part of the mScarlet minicircle containing mScarlet (only the C-terminal part is shown), followed by the sequence coding for an amino-acid linker, followed by one extra nucleotide that is necessary when targeting introns of frame 1 (circled in red), followed by a splice donor site.

FIG. 17. Experimental workflow of the generation of a pool of cells in which each cell harbours multiple tagged genes in HAP1 cells using two sgRNA libraries targeting different frames. a. Hap1 cells are transduced with a sgRNA library targeting intron of frame 0. Transduction is done at a low multiplicity of infection (MOI) to ensure that most cells are transduced with either none or just one virus particle. Two days after transduction, cells are treated with puromycin to eliminate cells that were not transduced and did not receive an intron targeting sgRNA together with a puromycin resistance gene. Transduced and selected cells are then transfected with a minicircle construct for targeting the intron frame 0 and a plasmid expressing Cas9 and a generic sgRNA targeting and linearizing the minicircle construct. In a small fraction of cells the linearized minicircle gets integrated at the intron-targeting sgRNA site as a synthetic exon, leading to tagging of the gene. Cells expressing tagged proteins are sorted to obtain a cell pool, where in every cell a different gene is tagged, depending in the expressed sgRNA. To tag multiple genes, the cell pool is then transduced with another sgRNA library targeting introns of frame 1, again at a low MOI. Two days after transduction, cells are treated with blasticidin to eliminate cells that were not transduced and did not receive an intron targeting sgRNA together with a blasticidin resistance gene. Transduced and selected cells are then transfected with a minicircle construct for targeting the intron frame 1 and a plasmid expressing Cas9 and a generic sgRNA targeting and linearizing the minicircle construct. In a small fraction of cells, the linearized minicircle gets integrated at the intron-targeting sgRNA site as a synthetic exon, leading to tagging of the second gene. Now double GFP and mScarlet+ cells are sorted to obtain a cell pool, where in every cell two genes are tagged. Cells are then transduced again to stably integrate and express further fluorescent proteins as structural makers or to further diversify the cellular phenotype of the pool.

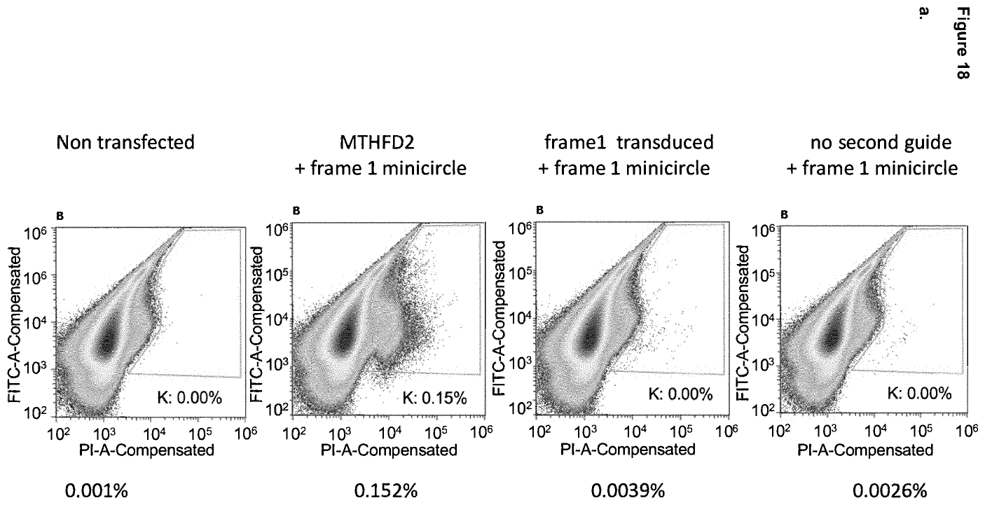

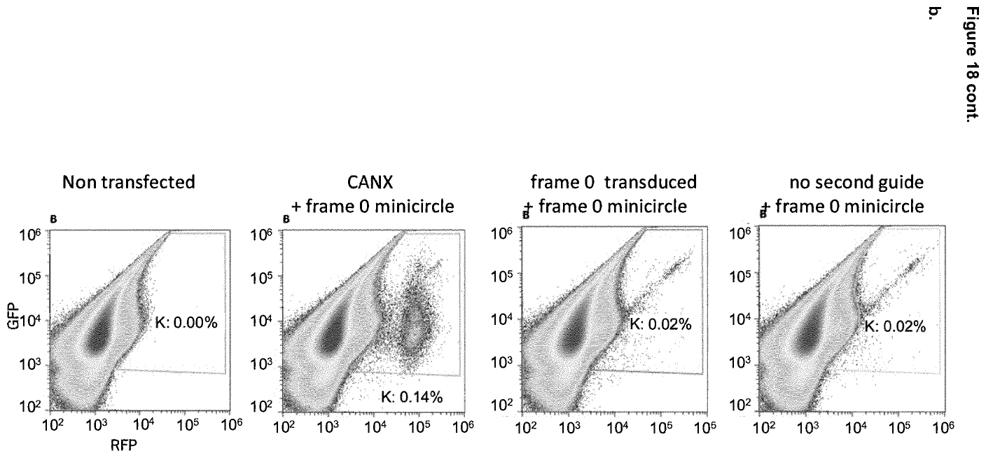

FIG. 18. FACS enrichment. a. FACS sorting of GFP and mScarlet positive cells after the second round of intron tagging, seven days after the transfection with the frame 1 minicircle and the plasmid expressing Cas9 and the generic sgRNA. The panel on the left is the negative control and shows cells that were not transfected. The second panel is the positive control and shows cells that were cotransfected with an intron targeting sgRNA targeting a frame 1 intron of MTHFD2 together with the frame 1 minicircle and the plasmid expressing Cas9 and the generic sgRNA. The third panel shows cells that were transduced with the frame 1 library and transfected with the frame 1 minicircle and the plasmid expressing Cas9 and the generic sgRNA. The fourth panel shows cells that were not transduced for a second time, but transfected with the frame 1 minicircle and the plasmid expressing Cas9 and the generic sgRNA. b. FACS sorting of GFP and mScarlet positive cells after intron tagging with the same library twice. Seven days after the transfection of the second round of intron tagging with the frame 0 minicircle and the plasmid expressing Cas9 and the generic sgRNA. The panel on the left is the negative control and shows cells that were not transfected. The second panel is the positive control and shows cells that were cotransfected with an intron targeting sgRNA targeting a frame 0 intron of CANX together with the frame 0 mScarlet minicircle and the plasmid expressing Cas9 and the generic sgRNA. The third panel shows cells that were transduced with the frame 0 library for a second time and transfected with the frame 0 mScarlet minicircle and the plasmid expressing Cas9 and the generic sgRNA. The fourth panel shows cells that were not transduced for a second time, but transfected with the frame 0 mScarlet minicircle and the plasmid expressing Cas9 and the generic sgRNA.

FIG. 19. Measuring sgRNA abundance in the double tagged cell pool to identify tagged genes present in the pool. a. Genomic DNA of the cell pool is isolated and used as a template for PCR amplification of sgRNA containing fragments. PCR amplicons are analyzed by next-generation sequencing and the obtained sequencing reads are mapped to the two sgRNA libraries that were used for generating the cell pool. b. List of the 30 most abundant sgRNAs in the pool that map to the frame 0 library. c. List of the 30 most abundant sgRNAs in the pool that map to the frame 1 library.

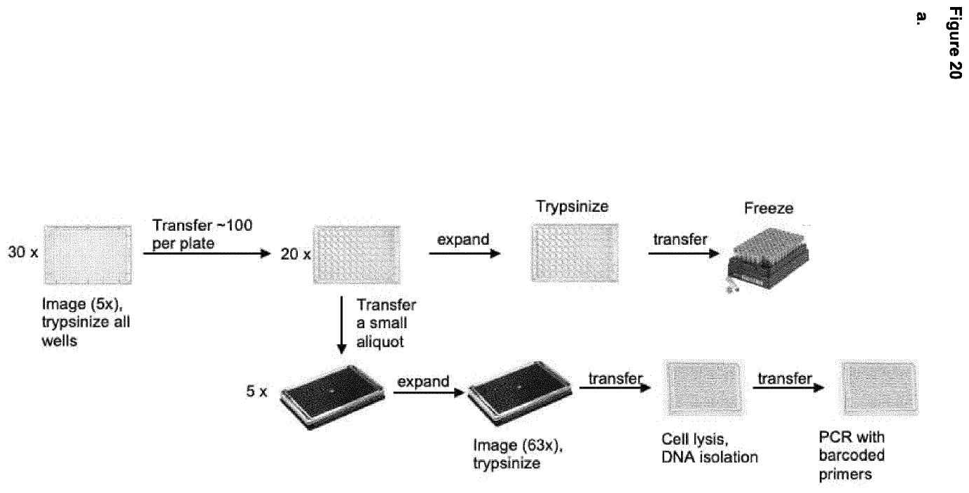

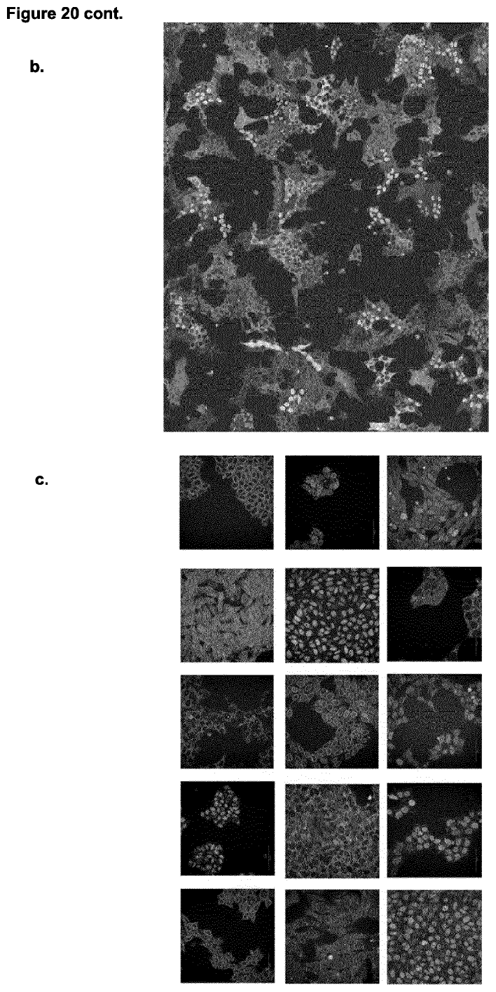

FIG. 20. a. Exemplary workflow for isolation, imaging and genotyping of hundreds to thousands of clonal cell lines from the double tagged cell pool. Single cells are seeded by single cell dilution into 384-well plates. After clonal expansion of cells, wells containing single colonies are transferred to a 96-well for further clonal expansion and to a 384-well imaging plate. Clonal cell lines in the imaging plates are imaged using a high-content fluorescence microscope multiple times over the course of 3 days. Cells on the imaging plates are then used for identifying the intron-targeting sgRNAs in the different clones indicating the tagged proteins. For that, cells are lysed and extracted DNA is used as a template for PCR amplification of the sgRNA containing fragments. PCR is performed using barcoded primers to enable pooling and next-generation sequencing of all reactions after PCR and to determine the sgRNA abundance in each well corresponding to each clone. Expanded clones on the 96-well plate are eventually harvested and clones can be frozen to generate large clone collections and to potentially re-pool selected clones. b. Microscopic image of the cell pool where in every cell two different genes are tagged, depending on the two intron-targeting sgRNAs expressed by that cell and every cell expresses two additional structural markers to aid cell segmentation and one fluorescent marker to diversify the pool. c. Exemplary microscopic images of clonal cell lines isolated from the cell pool using the workflow described above. Images of the clones together with the information on the two tagged proteins in each clone are used for training a model for automated recognition of clones in a pool.

FIG. 21. A simple classification model can easily discriminate 100 isolated clones. As a proof of concept, if single cells can be discriminated and if they can be assigned to the correct clone, a model was trained using images from 100 clonal cell lines. Using the structural markers expressed in every cell, the Cellpose algorithm was used to segment cells and obtain images of ˜20,000 single cells for further image analysis. CellProfiler was used to then extract ˜700 features such as intensity, granularity, texture, etc. Features can be determined separately for the nucleus and cytoplasm of the cell. Using all features or parameters of a cell, a random forest model was used to predict the identity of the clone in a separate set of test images of single cells that were not used for training. Images of the train and test set are taken from the same clone, but different timepoints and different fields of view in the well.

### REFERENCE EXAMPLE 1: EMPLOYED METHODS

Generation of an Intron-Targeting sgRNA Library

To design an intron-targeting sgRNA library for metabolic enzymes and epigenetic modifiers a list of 2,889 genes was generated by combining a published list of all classic metabolic enzymes (see, Corcoran (2017) Am J Physiol Renal Physiol 312, F533-F542), most genes in a human CRISPR metabolic gene knockout library (see; Birsoyv (2015) Cell 162, 540-51) as well as genes annotated with the GO terms “Histone modification”, “DNA methylation” or “DNA demethylation”. Then, the Ensembl BioMart data mining tool was used to obtain chromosomal coordinates of introns of the primary transcripts of those genes and only those introns were selected where integration of the donor plasmid does not lead to frameshift mutations after splicing, since the donor plasmid starts with a full codon and is not compatible to all exon-exon junctions. Using Ensembl BioMart this filtering was done by only selecting introns that are preceded by an exon with the attribute “End phase=0”. The GuideScan (Perez, 2017, Nat Biotechnol 35, 347-349) was then used to obtain the top 20 guides for each selected intronic region based on the GuideScan cutting efficiency score. Those 20 guides were then ranked based on a combined on- and off-target score using the scores provided by GuideScan. For genes that have only one intron that can be targeted, up to three sgRNAs per intron were selected, for genes with two or three introns that can be targeted, up to 2 sgRNAs per intron were selected and for genes that have more than three introns that can be targeted, the top ranked sgRNA of each intron was selected. Using that strategy, 14,049 sgRNAs targeting 11,614 introns of 2,387 genes were selected. In addition, 75 non-targeting sgRNAs from the human Brunello CRISPR KO library (Doench, 2016, Nat Biotechnol 34, 184-191) were added to the library. For cloning of the library into the CROPseq-Guide-Puro vector16 (Addgene #86708) using Gibson Assembly, adapter sequences were added to the sgRNA sequences and 74 nucleotide oligos were ordered as an oligo pool (Twist Biosciences). Additional adapters were added to the pooled oligos by PCR (8 cycles, NEB Q5) to generate fragments with a size of 140 nucleotides that were purified (QIAGEN MinElute PCR Purification) before being used for Gibson Assembly. The vector was digested with BsmBI (NEB), size-selected using agarose gel electrophoresis and gel purified (QIAGEN QIAquick Gel Extraction Kit) followed by an additional column purification (QIAGEN QIAquick PCR Purification Kit). 4 Gibson Assembly reactions (10 μl NEBuilder HiFi DNA Assembly, 60 ng vector, 10 ng insert) were prepared and incubated at 50° C. for 45 minutes. Reactions were pooled and purified (QIAGEN MinElute PCR Purification) before being used for transformation in Lucigen Endura electrocompetent bacteria (four reactions, 25 μl each). Bacteria were plated on four 245×245×25 mm Bioassay dishes and dilution plates (1:10,000) and incubated at 32° C. for 16 h. Cells were scraped off the plates and plasmid DNA was extracted using multiple QIAGEN Plasmid Plus Midi kits. Library coverage was 211× and was estimated based on the number of colonies on the dilution plates.

### Cloning

The GFP-donor plasmid with the coding sequence of EGFP flanked by generic sgRNA targeting sites, splice acceptor and splice donor sites and 20 amino acid linkers was assembled from 4 fragments using Gibson Assembly to generate a donor plasmid that is similar in design to a previously published donor plasmid that can be used for intron tagging; see Feldman (2019) Cell 179, 787-799 e17. The DNA fragment with a 25 nucleotide overlap to the pUC19 vector and 32 nucleotide overlap to the N-terminus of EGFP was generated from overlapping oligos (Sigma) and comprises a generic sgRNA targeting site that is not present in the human genome (He, 2016, Nucleic Acids Res 44, e85) followed by a splice acceptor site (Guzzardo, 2017, Sci Rep 7, 16770) and a flexible 20 amino acid glycine-serine linker. This fragment is followed by a fragment with the coding sequence of EGFP without a start or stop codon that was generated by PCR. The third fragment has a 27 nucleotide overlap to the C-terminus of EGFP and a 25 nucleotide overlap to the pUC19 vector and was generated from overlapping oligos (Sigma) and comprises a flexible 20 amino acid glycine-serine linker followed by a splice donor site (Guzzardo, 2017, loc, cit) the generic sgRNA targeting site. The pUC19 vector was linearized by PCR for Gibson Assembly (NEBuilder HiFi DNA Assembly) with the other three fragments.

The pX330 plasmid expressing Cas9 and the generic sgRNA targeting the donor plasmid was generated by digesting pU6-(Bbsl)_CBh-Cas9-T2A-mCherry (Addgene #64324; see also Chu, 2015, Nat Biotechnol 33, 543-8) with Bbsl followed by ligation with an annealed oligo duplex as described before; see, Ran (2013), Nat Protoc 8, 2281-2308. mCherry was replaced with a Blasticidin resistance (BSD) using Gibson Assembly.

### Pooled Protein Tagging

For the generation of lentiviral particles, HEK293T cells were transiently transfected with the intron-targeting library and packaging plasmids psPAX2, pMD2.G using PEI transfection. After 12 h the media was replaced with IMDM supplemented with 10% FBS and P/S. Viral supernatant was collected 48 h after transfection and stored at −80° C. HAP1 cells were transduced with virus and selected with puromycin for three days. Multiplicity of infection (MOI) was 0.2 and transduction was done at a coverage of 500×. After puromycin selection, cells were grown for one day in media without puromycin before being seeded for transfection (8 million cells per 15 cm dish, 48 million cells in total). One day after seeding, each dish was co-transfected with 20 μg pX330 expressing Cas9-BSD and the generic sgRNA and 10 μg EGFP donor plasmid with 90 μl Turbofection in 2.5 ml OptiMEM as described by the manufacturer. Transfection efficiency was approximately 10% as determined by a transfection done in parallel with pX330 Cas9-mCherry and the EGFP donor plasmid using the same ratio. The next day, cells were subjected to a transient selection using Blasticidin (10 μg/ml) for 24 h. After selection, cells were maintained in full media without Blasticidin and sorted five days after transfection by flow cytometry using a Sony Cell Sorter SH800ZD. 0.03% cells were GFP-positive and in total 24,300 of those GFP-positive cells were sorted and the cell population was expanded for 7 days before DNA was isolated to determine sgRNA abundance in the cell population.

### NGS Sequencing

In order to generate an NGS library, genomic DNA from one million cells of the GFP positive cell population was isolated and the sgRNA region was amplified by PCR (two reactions using 500 ng genomic DNA, NEB Q5 high-fidelity Polymerase). Illumina adapter ligation and sequencing were done by a commercial sequencing service. To determine sgRNA abundance, sgRNA sequences were extracted from NGS reads using Cutadapt and sgRNA read counts were determined using the MAGeCK count function to match the extracted reads to the sgRNA library. Of the 14,049 sgRNA in the library we considered 1,777 as highly enriched as these sgRNAs accounted for 90% of the obtained sequencing reads while the majority of sgRNAs was not detectable anymore. The remaining 10% of sequencing reads comprise an additional 1,622 sgRNAs, which we do not consider as enriched, as each of them is only supported by a few sequencing reads that might be the result of cells being transduced with two sgRNAs or the result of off-target integration and expression of the GFP-tag. Our library also includes 75 nontargeting sgRNAs making up 0.53% of the sgRNAs in our library. As expected, they are depleted in the pool of GFP-positive, making up 0.15% of the sequencing reads with only 3 non-targeting sgRNAs among the 1,777 sgRNAs we consider enriched.

### Isolation, Imaging and Sequencing of Clonal Cell Lines

To obtain clonal cell lines, cells were seeded at a concentration of 0.7 cells per well in 96-well cell culture plates. After 9 days of clonal expansion, 768 colonies were harvested using trypsin and cell suspensions were transferred in equal amounts to eight 96-well imaging plates (Perkin Elmer CellCarrier Ultra) and eight corresponding 96-well cell culture plates. After 24 h, cells on the imaging plates were imaged on a Perkin Elmer Opera Phenix High Content Screening System (5 fields of view per well, 63× water-immersion objective, confocal mode, excitation: 488 nm, emission: nm, 700 ms). Images were processed using Cell Profiler. To identify the intron-targeting sgRNAs expressed in imaged cells, multiplexed amplicon sequencing of the sgRNA regions was performed in the corresponding clones on the eight 96-well cell culture plates. Cells were lysed and cell lysates were used for PCR to amplify the sgRNA region in each clone using barcoded primers flanking the sgRNA region (36 different 5-mers added to the 5′end of the forward primer and 24 different 5-mers added to the 5′end of the reverse primer, 768 of all possible 864 combinations were used). PCR reactions were pooled and column purified before being send for sequencing by a commercial sequencing service. NGS reads were demultiplexed using Cutadapt (see Martin, M. EMBnet.journal, [S.I.], v. 17, n. 1, p. pp. 10-12, May 2011) and sgRNA read counts for each individual well were obtained using MAGeCK (see, Li (2014) Genome Biol 15, 554 (2014) . . . . For further analysis clones were excluded, for which either no cells in any of the 5 fields of view that were imaged were observed, no sequencing reads for the corresponding well were observed or for which polyclonal cell populations as determined by imaging or detection of multiple sgRNAs per well were observed. Using that strategy, images of 335 clones were obtained for which the expressed intron-targeting sgRNA corresponding to the tagged protein could be identified.

### Comparison of Subcellular Localization to the Human Protein Atlas

Comparison of subcellular protein localizations of GFP-tagged protein in 335 clones to the localization patterns as annotated on The Human Protein Atlas was done as described previously for the comparison of N- or C-terminally GFP-tagged proteins to IF-based annotations on the Human Protein Atlas, see Stadler (2013) Nat Methods 10, 315-23. Briefly, the overlap was defined as ‘identical’ if one or multiple main and additional localizations were the same in the intron-tagged clone compared to The Human Protein Atlas, ‘similar’ if one localization is the same in the clone compared to The Human Protein Atlas with additional localization(s) observed either in the clone or on The Human Protein atlas or ‘dissimilar’ if there were no common subcellular localization patterns. Extended localization annotations such as nucleoplasm, nuclear speckles or nucleoli that were considered as “nuclear” were not taken into account.

### Live Cell Imaging

Live cell imaging was performed on a PerkinElmer Opera Phenix microscope with excitation laser 488 nm, and emission filter 500-550 nm, 700 ms.

### In Situ Sequencing

Identification of the expressed sgRNAs by in situ sequencing was performed by following and modifying published protocols, see, e.g., Feldman (2019) loc. cit; Ke (2013) Nat Methods 10, 857-60; and Larsson (2010) Nat Methods 7, 395-7.

After live-cell imaging after treatment with MTX or dBET6, cells were fixed with 4% paraformaldehyde for 30 minutes, washed with PBS, permeabilized with 70% ethanol for 30 minutes and washed with PBS-T (PBS+0.05% Tween-20) twice. Reverse transcription mix (1× RevertAid RT buffer, 250 UM dNTPs, 0.2 mg/mL BSA, 1 UM RT primer, 0.8 U/mL Ribolock RNase inhibitor, and 4.8 U/mL RevertAid H minus reverse transcriptase) was added to the sample and incubated for 16 hours at 37° C. Following reverse transcription, cells were washed 5 times with PBS-T and post-fixed with 3% paraformaldehyde and 0.1% glutaraldehyde for 30 minutes at room temperature and washed 5 times with PBS-T. Cells were incubated in a padlock probe and extension-ligation reaction mix (1× Ampligase buffer, 0.4 U/mL RNase H, 0.2 mg/mL BSA, 100 nM padlock probe, 0.02 U/mL KlenTaq polymerase, 0.5 U/mL Ampligase and 50 nM dNTPs) for 5 minutes at 37° C. and 90 minutes at 45° C., and then washed 2 times with PBS-T. Circularized padlocks were amplified with rolling circle amplification mix (1× Phi29 buffer, 250 UM dNTPs, 0.2 mg/mL BSA, 5% glycerol, and 1 U/mL Phi29 DNA polymerase) at 30° C. for 4 hours. Rolling circle amplicons were prepared for sequencing by hybridizing a mix containing sequencing primer oSBS_CROP-seq (1 UM primer in 2×SSC+10% formamide) for 30 minutes at room temperature. Barcodes were read out using sequencing-by-synthesis reagents from the Illumina NextSeq 500/550 kit v2 (Illumina 15057934). First, samples were washed with incorporation buffer (NextSeq 500/550 buffer cartridge, position 35) and incubated for 4 minutes in incorporation mix (NextSeq 500/550 reagent cartridge, position 31) at 60° C. Samples were then washed with incorporation buffer (4 washes, 60° C. for 4 minutes at the last wash) and placed in scan mix (NextSeq 500/550 reagent cartridge, position 30) for imaging. Imaging was performed on a PerkinElmer Opera Phenix microscope with excitation laser: 561 nm, emission filter: 570-630, 500 ms; excitation laser: 640 nm, emission filter: 650-760 nm, 500 ms using a 63× water immersion objective, confocal mode. Based were detected as follows: Base T: signal in 561 channel; Base C: signal in 640 channel, Base A: (weaker) signal in both channels, Base G: no signal. Following each imaging cycle, samples were washed with the cleavage mix (NextSeq 500/550 reagent cartridge, position 29) once followed by incubation with cleavage mix for 4 minutes at 60° C. to remove dye terminators. Samples were washed 5 times with incorporation buffer before starting the next cycle.

### Primer Sequences Employed:

NGS Sequencing
CROP-seq sgRNA amplicon fwd ATCTTGTGGAAAGGACGAAACACC (SEQ ID NO: 1)
CROP-seq sgRNA amplicon rev tgtctcaagatctagttacgcca (SEQ ID NO: 2)
in situ sequencing
ORT_CROPseq
G+AC+TA+GC+CT+TA+TT+TTAACTTGCTAT (Feldman et al.) (SEQ ID NO: 3)
oPD_CROPseq
/5Phos/gttttagagctagaaatagcaagCTCCTGTTCGACACCTACCCACCTCATCCCACTCT
TCAaaaggacgaaacaccg
(Feldman et al.) (SEQ ID NO: 4).
The term ″5Phos″ specifies, in particular, a single phosphate on the 5′ of the
primer instead of a free OH group. Thus the ″G″ might be considered a ″GMP″ if it
was a free nucleotide.
oSBS_CROPseq
CACCTCATCCCACTCTTCAaaaggacgaaacaccg (SEQ ID NO: 5) Feldman et al.
multiplexed amplicon sequencing barcodes
FWD1_1
GAGAAATCTTGTGGAAAGGACGAAACAC (SEQ ID NO: 6)

| | |
|---|---|
| FWD1_2 | CAAGAATCTTGTGGAAAGGACGAAACAC (SEQ ID NO: 7) |
| FWD1_3 | GAACAATCTTGTGGAAAGGACGAAACAC (SEQ ID NO: 8) |
| FWD1_4 | CCATAATCTTGTGGAAAGGACGAAACAC (SEQ ID NO: 9) |
| FWD1_5 | GTTAGATCTTGTGGAAAGGACGAAACAC (SEQ ID NO: 10) |
| FWD1_6 | ACTCGATCTTGTGGAAAGGACGAAACAC (SEQ ID NO: 11) |
| FWD1_7 | TGTTGATCTTGTGGAAAGGACGAAACAC (SEQ ID NO: 12) |
| FWD1_8 | AGGTCATCTTGTGGAAAGGACGAAACAC (SEQ ID NO: 13) |
| FWD1_9 | AGGAAATCTTGTGGAAAGGACGAAACAC (SEQ ID NO: 14) |
| FWD1_10 | ACAGAATCTTGTGGAAAGGACGAAACAC (SEQ ID NO: 15) |
| FWD1_11 | AGACAATCTTGTGGAAAGGACGAAACAC (SEQ ID NO: 16) |
| FWD1_12 | TGGTAATCTTGTGGAAAGGACGAAACAC (SEQ ID NO: 17) |
| FWD2_1 | CTAGGATCTTGTGGAAAGGACGAAACAC (SEQ ID NO: 18) |
| FWD2_2 | CGATGATCTTGTGGAAAGGACGAAACAC (SEQ ID NO: 19) |
| FWD2_3 | TTCGCATCTTGTGGAAAGGACGAAACAC (SEQ ID NO: 20) |
| FWD2_4 | GGTTCATCTTGTGGAAAGGACGAAACAC (SEQ ID NO: 21) |
| FWD2_5 | CCGAAATCTTGTGGAAAGGACGAAACAC (SEQ ID NO: 22) |
| FWD2_6 | TCGGAATCTTGTGGAAAGGACGAAACAC (SEQ ID NO: 23) |
| FWD2_7 | AAGCAATCTTGTGGAAAGGACGAAACAC (SEQ ID NO: 24) |
| FWD2_8 | TCCTAATCTTGTGGAAAGGACGAAACAC (SEQ ID NO: 25) |
| FWD2_9 | AATGGATCTTGTGGAAAGGACGAAACAC (SEQ ID NO: 26) |
| FWD2_10 | GCATGATCTTGTGGAAAGGACGAAACAC (SEQ ID NO: 27) |
| FWD2_11 | TATGCATCTTGTGGAAAGGACGAAACAC (SEQ ID NO: 28) |
| FWD2_12 | CCTGTATCTTGTGGAAAGGACGAAACAC (SEQ ID NO: 29) |
| FWD3_1 | GTGGAATCTTGTGGAAAGGACGAAACAC (SEQ ID NO: 30) |
| FWD3_2 | CTGCAATCTTGTGGAAAGGACGAAACAC (SEQ ID NO: 31) |
| FWD3_3 | ACCAAATCTTGTGGAAAGGACGAAACAC (SEQ ID NO: 32) |
| FWD3_4 | TGCGAATCTTGTGGAAAGGACGAAACAC (SEQ ID NO: 33) |
| FWD3_5 | TGTCAATCTTGTGGAAAGGACGAAACAC (SEQ ID NO: 34) |
| FWD3_6 | TAGAGATCTTGTGGAAAGGACGAAACAC (SEQ ID NO: 35) |
| FWD3_7 | TACCGATCTTGTGGAAAGGACGAAACAC (SEQ ID NO: 36) |
| FWD3_8 | TTGTGATCTTGTGGAAAGGACGAAACAC (SEQ ID NO: 37) |
| FWD3_9 | ACACCATCTTGTGGAAAGGACGAAACAC (SEQ ID NO: 38) |
| FWD3_10 | ACCTTATCTTGTGGAAAGGACGAAACAC (SEQ ID NO: 39) |
| FWD3_11 | CTCTAATCTTGTGGAAAGGACGAAACAC (SEQ ID NO: 40) |
| REV1_A | GGCAATGTCTCAAGATCTAGTTACGCCA (SEQ ID NO: 42) |
| REV1_B | AACGATGTCTCAAGATCTAGTTACGCCA (SEQ ID NO: 43) |
| REV1_C | GTCCATGTCTCAAGATCTAGTTACGCCA (SEQ ID NO: 44) |
| REV1_D | TGAAGTGTCTCAAGATCTAGTTACGCCA (SEQ ID NO: 45) |
| REV1_E | GTACGTGTCTCAAGATCTAGTTACGCCA (SEQ ID NO: 46) |
| REV1_F | ACGTGTGTCTCAAGATCTAGTTACGCCA (SEQ ID NO: 47) |
| REV1_G | CAACCTGTCTCAAGATCTAGTTACGCCA (SEQ ID NO: 48) |
| REV1_H | CACTTTGTCTCAAGATCTAGTTACGCCA (SEQ ID NO: 49) |
| REV2_A | CGTAATGTCTCAAGATCTAGTTACGCCA (SEQ ID NO: 50) |
| REV2_B | GGTGATGTCTCAAGATCTAGTTACGCCA (SEQ ID NO: 51) |
| REV2_C | CCTCATGTCTCAAGATCTAGTTACGCCA (SEQ ID NO: 52) |
| REV2_D | ATGAGTGTCTCAAGATCTAGTTACGCCA (SEQ ID NO: 53) |
| REV2_E | ATCCGTGTCTCAAGATCTAGTTACGCCA (SEQ ID NO: 54) |
| REV2_F | GACTGTGTCTCAAGATCTAGTTACGCCA (SEQ ID NO: 55) |
| REV2_G | TCTCCTGTCTCAAGATCTAGTTACGCCA (SEQ ID NO: 56) |
| REV2_H | GCTAATGTCTCAAGATCTAGTTACGCCA (SEQ ID NO: 57) |
| REV3_A | CTTGATGTCTCAAGATCTAGTTACGCCA (SEQ ID NO: 58) |
| REV3_B | GGATATGTCTCAAGATCTAGTTACGCCA (SEQ ID NO: 59) |
| REV3_C | AGTAGTGTCTCAAGATCTAGTTACGCCA (SEQ ID NO: 60) |
| REV3_D | GATCGTGTCTCAAGATCTAGTTACGCCA (SEQ ID NO: 61) |
| REV3_E | AGCTGTGTCTCAAGATCTAGTTACGCCA (SEQ ID NO: 62) |
| REV3_F | CTTCCTGTCTCAAGATCTAGTTACGCCA (SEQ ID NO: 63) |
| REV3_G | ATTGCTGTCTCAAGATCTAGTTACGCCA (SEQ ID NO: 64) |
| REV3_H | GTGCTTGTCTCAAGATCTAGTTACGCCA (SEQ ID NO: 65) |

GFP generic donor plasmid (relevant part)
atgttctttcctgcgttatcccctggagatcgagtgccgcatcacCGGCTATTGGTCTTACTGACATCCACT
TTGCCTTTCTCTCCACAGggtggcggtggctcgggcggtggtgggtccggtggcggcggatctggcggtggt
ggatccgtgagcaagggcgaggagctgttcaccggggtggtgcccatcctggtcgagctggacggcgacgtaaacggc
cacaagttcagcgtgtccggcgagggcgagggcgatgccacctacggcaagctgaccctgaagttcatctgcaccacc
ggcaagctgcccgtgccctggcccaccctcgtgaccaccctgacctacggcgtgcagtgcttcagccgctaccccgacc
acatgaagcagcacgacttcttcaagtccgccatgcccgaaggctacgtccaggagcgcaccatcttcttcaaggacga
cggcaactacaagacccgcgccgaggtgaagttcgagggcgacaccctggtgaaccgcatcgagctgaagggcatc
gacttcaaggaggacggcaacatcctggggcacaagctggagtacaactacaacagccacaacgtctatatcatggcc
gacaagcagaagaacggcatcaaggtgaacttcaagatccgccacaacatcgaggacggcagcgtgcagctcgccg
accactaccagcagaacacccccatcggcgacggccccgtgctgctgcccgacaaccactacctgagcacccagtcc
gccctgagcaaagaccccaacgagaagcgcgatcacatggtcctgctggagttcgtgaccgccgccgggatcactctc
ggcatggacgagctgtacaagggcggcggtggatccggtggcggcggatctggcggtggaggttcgggtggcggtgg
gtcgggcaagGTAAGTATCAATTAGAAGACGAATTCCCGgtgatgcggcactcgatctcGAATTCt
tacagacaagctgtgaccgtctccgggagctgcatgtgtcagaggttttcaccgtcatcaccgaaacgcgcgagacgaa
agggcctcgtgatacgcctatttttataggttaatgtcatgataataatgg (SEQ ID NO: 66)

### REFERENCE EXAMPLE 2: GENERATION OF A CELL POOL COMPRISING PROTEIN TAGGING AND BEING EMPLOYED FOR CELLULAR IMAGING AND IN SITU SEQUENCING-NOVEL MEANS AND METHODS FOR REAL TIME DRUG TESTING AND/OR SCREENING

The present invention relates to the provision of a large cell pool that comprises individual intro-tagged proteins.

As illustrative, non limiting example of the means and methods of the present invention, a pooled GFP (green fluorescent protein)-intron-tagging of metabolic enzymes is provided herein. As provided herein, a CRISPR/Cas9 mediated intron tagging approach is employed to generate a large pool of cells herein with more than 900 tagged proteins, wherein each cell comprises one tagged protein, i.e. a “one protein per cell” approach is provided. The inventive means and methods of the present invention offer the following advantages, namely that (i) by designing the sgRNA target genes can be chosen as desired (ii) by designing the sgRNA different introns for the same genes can be chosen, allowing to avoid tagging within functionally important domains and (iii) that very homogenous distributions of cells can be generated with roughly equal numbers of clones for each targeted protein.

A second key aspect of the inventive method is the application of in situ sequencing. Following exposure of the inventive cell pool to molecules to be screened (for example. drugs and/or pharmacologically relevant molecules), some cells respond with changes in protein localization or in protein abundance (measured by fluorescence microscopy of the GFP tag fused to the protein). The application of a CROP-seq vector as provided and illustrated herein for the intron-targeting sgRNA library allows for in situ sequencing in order to identify the tagged intron. In order to render this compatible with the provided illustrative GFP tagged cell pool, the in situ sequencing protocol was adopted to a two color system

Accordingly, a CRISPR-Cas9 based intron tagging is employed herein to generate cell pools expressing hundreds of labeled/tagged-fusion proteins at endogenous levels, to monitor drug effects on protein levels and/or to localization by time-lapse microscopy. Furthermore, within the means and methods of the present invention is the identification of targeted introns by in situ sequencing. Accordingly, the means and methods of the present invention provide for a pooled protein tagging approach allowing for the localization and even (expression) levels of hundreds of proteins in individual cells in real time; see also illustrative FIG. 2a

In context of the present invention, 2,889 genes were selected to be targeted comprising all classic metabolic enzymes and epigenetic modifiers; see Corcoran (2017). Am J Physiol Renal Physiol 312, F533-F542; Birsoy (2015), Cell 162, 540-51. For the 2,387 genes from this set that harbor targetable introns in the selected reading frame, a library comprising 14,049 sgRNAs targeting 11,614 introns (FIG. 2b) was designed. To generate a pool of GFP-tagged cells, HAP1 cells were transduced with that sgRNA library followed by co-transfection with a GFP donor plasmid and a plasmid expressing Cas9 and the donor-targeting sgRNA. It was enriched for transfected cells using blasticidin for 24 h and sorted GFP-positive cells 6 days after transfection (FIG. 2c). NGS-based sgRNA amplicon sequencing of the pool of GFP-positive cells identified 1,777 sgRNAs targeting 1,650 introns of 953 genes as highly enriched in the GFP-positive cell pool (FIG. 2d). Compared to genes for which intron targeting sgRNAs did not result in isolation of GFP positive cells, successfully targeted genes have higher average expression in HAP1 cells (FIG. 2e). Clonal cell lines were then isolated from the pool by single cell dilution and clonal expansion, in which GFP localization was imaged (FIG. 2f) and sgRNAs were identified indicating the tagged proteins by an NGS-based multiplex sgRNA amplicon sequencing strategy. After removing cell lines in which more than one sgRNA was present, 362 clonal lines were obtained. The main localization of GFP-tagged proteins in the majority of our cell lines was either cytoplasmic, nuclear or mitochondrial, with some proteins showing a typical ER localization pattern. These localization corresponded to the antibody-based annotations in the Human Protein Atlas (Thul, 2017, Science 356) in 72% of the clones (FIG. 2f), and for 40 GFP-tagged proteins in the clonal cell lines no previous localization data is available.

It was reasoned that the highly diverse pool of cells expressing GFP-tagged proteins can be used to identify compounds that change protein levels or localization of any of the tagged proteins. Therefore, the cell pool was treated with the BRD4-targeting PROTAC dBET6 (Winter (2017). Mol Cell 67, 5-18 e19) and high-content live cell imaging was used to track protein dynamics of GFP-tagged proteins over 9 hours in approximately 7,000 cells in a single well on a 384-well plate (FIG. 6a). A drastic loss of GFP signal was observed in selected clones already 1 h after compound treatment. These clones had a nuclear GFP localization pattern with few selected foci, compatible with the known phase separation behavior of BRD4 (FIG. 6b); see also Sabari (2018) Science 361. The application of the CROP-seq vector16 that expresses the sgRNA sequence in a polyadenylated mRNA transcript enables the cell-specific identification of the targeted intron by situ sequencing (see also Feldman, D. et al. Cell 179, 787-799 e17 (2019), Ke, R. et al. Nat Methods 10, 857-60 (2013), Larsson, C. et al. Nat Methods 7, 395-7 (2010). 17-19). To map targeted introns, the cell pool was fixed and a modified in situ sequencing protocol was developed to identify the sgRNA sequence integrated into individual cells in the pool. Based on the library diversity, eight cycles of nucleotide incorporation and imaging are sufficient to unambiguously assign sgRNA sequences (FIG. 6c). Application of this protocol to the cell pool confirmed that in clones with drastic loss of signal GFP was indeed targeted to BRD4 (FIG. 6d). Analysis of the entire cell pool revealed several other effects of the compound, including the loss of subnuclear localization patterns of MEAF1 and FUBP3, gain of nuclear foci of AKAP8 and SFPQ, and loss of nuclear intensity for UNG (FIG. 6e), none of which are identifiable by global proteomics profiling; see also Winter (2017), loc. cit.

It was then tested whether the cell pool also reveals complex cellular responses to compounds that act by conventional mechanisms. Therefore, the cell pool was treated with methotrexate (MTX), an antimetabolite impairing DNA and RNA synthesis and causing DNA damage by inhibiting tetrahydrofolate metabolism. Changes to the localizations of several proteins were observed in the cell pool (FIG. 10a), which could be further validated by applying MTX to the arrayed individual clones (FIG. 10b). Importantly, many of the findings are consistent with the known effects of the drug. For example, 24 h MTX treatment caused increased nuclear localization of GFP-tagged ACLY, a metabolic enzyme that has been shown to translocate to the nucleus in response to DNA damage; Sivanand (2017) Mol Cell 67, 252-265 e6. In cell lines expressing either GFP-tagged RPA1 or RPA2, which are part of a heterotrimeric DNA single-strand binding complex, the formation of nuclear foci was observed in response to treatment, presumably by the recruitment of the proteins to sites of DNA damage; see Raderschall (1999), Proc Natl Acad Sci USA 96, 1921-6. In addition to these predicted effects, a gain of nuclear signal for TPI1, NUDT21 and the transcription factor DR1, the disappearance of nuclear DNMT1 foci, and decreases of RUVBL1 and PADI1 protein was also noted. Importantly, some of the observations are supported by multiple clonal cell lines expressing the same GFP-tagged protein tagged at different intronic sites or by multiple proteins that are part of the same complex, increasing the confidence in the observed drug effects on different proteins.

The generation of targeted GFP tagged cell pools enables, inter alia, the identification of cellular drug responses by time lapse microscopy. Future applications of the present invention and corresponding uses, including deep learning and image recognition as well as direct in situ sequencing, will further accelerate the assignment of the targeted clones directly from screening well. Importantly, the low cost and fast timescales of imaging-based approaches enable applications both in large scale screening and in the rapid optimization of doses and response kinetics in a cellular system. This approach is especially useful for the discovery and development of PROTACs and molecular glue degraders, for which activity can easily be determined by the disappearance of the tagged protein, however we document herein also that the means and methods of the present invention can be employed to verify and/or confirm known drug actions and/or to discover new effects of known drugs. Importantly, intron tagging can easily be applied for other sets of genes beyond metabolic enzymes and potentially in a genome-wide manner to study protein dynamics at scale not only in response to drug treatment or other physiological perturbations.

### REFERENCE EXAMPLE 3—MINICIRCLE PLASMIDS

For protein tagging with an intron tagging strategy, a generic sgRNA is excising a fluorescent tag flanked by splice acceptor and donor sites from a generic donor plasmid.

This excision was done by cutting the donor plasmid twice, resulting in the fragment containing the coding sequence of the tag flanked by splice acceptor and a splice donor (FIG. 11A). However, another fragment containing the plasmid backbone is generated and this fragment is equally likely getting integrated at target sites as specified by an intron targeting sgRNA. If the plasmid backbone gets integrated, these target sites are not available anymore for integration of GFP containing plasmid, thereby lowering the editing efficiency (FIG. 11B). Furthermore, it was observed that in some cases the donor plasmid is cut only once, leading to integration of the entire linearized plasmid containing not only the coding sequence of the protein tag, but also the plasmid backbone containing sequences for plasmid amplification on bacteria (origin of replication, resistance gene etc., FIG. 11C). While cells with those integrations would still be GFP positive, it was observed that integration of the plasmid backbone can change levels of or correct localization of GFP-fusions. Here it is shown that using a minicircle that contains only the coding sequence of the protein tag flanked by splice acceptor and donor sites and has only one generic sgRNA target site to linearize the minicircle, significantly improves the intron tagging strategy by addressing both disadvantages of the current strategy (FIG. 11D).

To compare the conventional donor plasmid to a minicircle, it was attempted to tag CANX at intron 14 by using either a GFP donor plasmid containing two generic sgRNA sites or a GFP minicircle containing only one generic sgRNA sites and no plasmid backbone. A tagging rate of 3.0% was achieved when using the GFP donor plasmid as determined by analyzing transfected cells by flow cytometry (FIG. 12A). When using the GFP minicircle, the tagging rate increased approximately two-fold to 6.5% (FIG. 12B). To rule out that the observed increase is only resulting from a higher number of transfected fragments (GFP minicircle is 1 kb in length compared to the 3 kb GFP donor plasmid, which is why 200 ng of GFP minicircle contains three times as many molecules as 200 ng of GFP donor plasmid) a third sample was transfected with ⅓ the amount of donor plasmid (67 ng) and again an improved tagging rate of 5.7% was observed (FIG. 12C). Therefore, it was concluded that the improved tagging rate is not simply the result of a higher number of fragments that are present in the cell, but is due to the design of the minicircle, making integration of any plasmid backbone fragment impossible. Additionally, when using the minicircle it is not possible to integrate the GFP containing fragment together with the plasmid backbone as observed when the conventional donor plasmid is only cut once and linearized plasmid gets integrated. These integrations can result in lower expression levels of tagged genes and indeed less GFP-positive cells with intensity levels only slightly above the autofluorescence of cells were observed (note the very distinct GFP-positive population in the two minicircle samples compared to the population in the GFP donor plasmid sample).

In a second independent experiment, similar improvements when using the minicircle were observed (4-fold increase in GFP-positive cells when using the same amount of GFP minicircle DNA as GFP donor plasmid and 3-fold when using ⅓ the amount of minicircle DNA) but in this experiment the overall tagging rates were lower due to lower transfection efficiency (less than 10% of cells that were analyzed were transfected, FIG. 13A-C).

Minicircle DNA was produced with a commercial minicircle production kit (SBI MC-Easy™ Minicircle DNA Production Kit). First, a parental production plasmid was generated by cloning a DNA fragment starting with the generic sgRNA target site followed by a splice acceptor, a 20 amino acid linker sequence, the coding sequence of EGFP, another 20 amino acid linker sequence and a splice donor site into the pMC.BESPX-MCS1 production plasmid. The DNA fragment was generated by PCR using the GFP donor plasmid as a template, pMC.BESPX-MCS1 was digested with EcoRV and the fragment was integrated at the restriction site via Gibson Assembly. The E. coli producer strain ZYCY10P3S2T was transformed with the ligation reaction and clonal bacterial colonies were selected for isolation and sequencing of parental plasmid. A colony containing the correct parental plasmid was used for minicircle production as described by the manufacturer. In brief, bacteria were grown overnight in the provided growth media and induction media was added the next day to induce att recombination and parental plasmid backbone degradation. Minicircle DNA was isolated from bacterial pellets using multiple Qiagen Plasmid Plus Midi kits and the produced minicircle was analyzed by restriction enzyme digest and gel electrophoresis.

For intron tagging experiments, A549 cells were cells seeded in a 12-well plate and were co-transfected with 400 ng of the CROPseq plasmid expressing the intron-targeting sgRNA targeting intron 14 of the CANX gene, 400 ng of the pX330 plasmid expressing Cas9-mCherry and the donor-targeting sgRNA, together with 200 ng of the GFP donor plasmid or 200 ng GFP minicircle using Lipofectamine 3000 as described by the manufacturer. In samples with ⅓ of the amount of GFP minicircle, cells were cotransfected with 467 ng of the CROPseq plasmid with the intron-targeting sgRNA targeting intron 14 of the CANX gene, 467 ng of the pX330 plasmid expressing Cas9-mCherry and the donor-targeting sgRNA, and 67 ng GFP minicircle. To enrich for transfected cells, mCherry-positive cells were sorted 48 h after transfection and expanded for one week before GFP-positive cells were sorted. In an independent experiment a px330 plasmid expressing Cas9-BSD instead of Cas9-mCherry was used, cells were not enriched for transfected cells and GFP-positive cells were sorted 48 h after transfection.

Annotation and sequence of the parental GFP minicircle production plasmid:

| | |
|---|---|
| 1-738 | 32 consecutive ISce1 site |
| 782-816 | attB site |
| 852-871 | generic sgRNA target site |
| 872-874 | PAM site |
| 875-916 | splice acceptor |
| 917-976 | 20-amino-acid-linker |
| 977-1690 | EGFP |
| 1691-1753 | 21-amino-acid-linker |
| 1754-1776 | splice donor |
| 1812-1850 | attP site |
| 1937-2731 | Kanamycin resistance |
| 3034-5071 | EcoE1 origin |

Only the sequence between the attB and attP site circularizes and remains in the final GFP minicircle.

Only the part between the attB and attP sites was designed. The parental producer plasmid backbone is part of the commercial SBI MC-Easy™ Minicircle DNA Production Kit.

(SEQ ID NO: 67)
ACATTACCCTGTTATCCCTAGATACATTACCCTGTTATCCCAGATGACA
TACCCTGTTATCCCTAGATGACATTACCCTGTTATCCCAGATGACATTA
CCCTGTTATCCCTAGATACATTACCCTGTTATCCCAGATGACATACCCT
GTTATCCCTAGATGACATTACCCTGTTATCCCAGATGACATTACCCTGT
TATCCCTAGATACATTACCCTGTTATCCCAGATGACATACCCTGTTATC
CCTAGATGACATTACCCTGTTATCCCAGATGACATTACCCTGTTATCCC
TAGATACATTACCCTGTTATCCCAGATGACATACCCTGTTATCCCTAGA
TGACATTACCCTGTTATCCCAGATGACATTACCCTGTTATCCCTAGATA
CATTACCCTGTTATCCCAGATGACATACCCTGTTATCCCTAGATGACAT
TACCCTGTTATCCCAGATGACATTACCCTGTTATCCCTAGATACATTAC
CCTGTTATCCCAGATGACATACCCTGTTATCCCTAGATGACATTACCCT
GTTATCCCAGATGACATTACCCTGTTATCCCTAGATACATTACCCTGTT
ATCCCAGATGACATACCCTGTTATCCCTAGATGACATTACCCTGTTATC
CCAGATGACATTACCCTGTTATCCCTAGATACATTACCCTGTTATCCCA
GATGACATACCCTGTTATCCCTAGATGACATTACCCTGTTATCCCAGAT
AAACTCAATGATGATGATGATGATGGTCGAGACTCAGCGGCCGCGGTGC
CAGGGCGTGCCCTTGGGCTCCCCGGGCGCGACTAGTGAATTCAGATCTG
ATCCTGCGTTATCCCCTGGAGATCGAGTGCCGCATCACCGGCTATTGGT
CTTACTGACATCCACTTTGCCTTTCTCTCCACAGGGTGGCGGTGGCTCG
GGCGGTGGTGGGTCCGGTGGCGGCGGATCTGGCGGTGGTGGATCCGTGA
GCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAG
GGCGATGCCACCTACGGCAAGCTGACCCTGAAGTTCATCTGCACCACCG
GCAAGCTGCCCGTGCCCTGGCCCACCCTCGTGACCACCCTGACCTACGG
CGTGCAGTGCTTCAGCCGCTACCCCGACCACATGAAGCAGCACGACTTC
TTCAAGTCCGCCATGCCCGAAGGCTACGTCCAGGAGCGCACCATCTTCT
TCAAGGACGACGGCAACTACAAGACCCGCGCCGAGGTGAAGTTCGAGGG
CGACACCCTGGTGAACCGCATCGAGCTGAAGGGCATCGACTTCAAGGAG
GACGGCAACATCCTGGGGCACAAGCTGGAGTACAACTACAACAGCCACA
ACGTCTATATCATGGCCGACAAGCAGAAGAACGGCATCAAGGTGAACTT
CAAGATCCGCCACAACATCGAGGACGGCAGCGTGCAGCTCGCCGACCAC
TACCAGCAGAACACCCCCATCGGCGACGGCCCCGTGCTGCTGCCCGACA
ACCACTACCTGAGCACCCAGTCCGCCCTGAGCAAAGACCCCAACGAGAA
GCGCGATCACATGGTCCTGCTGGAGTTCGTGACCGCCGCCGGGATCACT
CTCGGCATGGACGAGCTGTACAAGGGCGGCGGTGGATCCGGTGGCGGCG
GATCTGGCGGTGGAGGTTCGGGTGGCGGTGGGTCGGGCAAGGTAAGTAT
CAATTAGAAGACGAATTCCATCTCTAGAGTCGACCCATGGGGGCCCGCC
CCAACTGGGGTAACCTTTGAGTTCTCTCAGTTGGGGGTAATCAGCATCA
TGATGTGGTACCACATCATGATGCTGATTATAAGAATGCGGCCGCCACA
CTCTAGTGGATCTCGAGTTAATAATTCAGAAGAACTCGTCAAGAAGGCG
ATAGAAGGCGATGCGCTGCGAATCGGGAGCGGCGATACCGTAAAGCACG
AGGAAGCGGTCAGCCCATTCGCCGCCAAGCTCTTCAGCAATATCACGGG
TAGCCAACGCTATGTCCTGATAGCGGTCCGCCACACCCAGCCGGCCACA
GTCGATGAATCCAGAAAAGCGGCCATTTTCCACCATGATATTCGGCAAG
CAGGCATCGCCATGGGTCACGACGAGATCCTCGCCGTCGGGCATGCTCG
CCTTGAGCCTGGCGAACAGTTCGGCTGGCGCGAGCCCCTGATGCTCTTC
GTCCAGATCATCCTGATCGACAAGACCGGCTTCCATCCGAGTACGTGCT
CGCTCGATGCGATGTTTCGCTTGGTGGTCGAATGGGCAGGTAGCCGGAT
CAAGCGTATGCAGCCGCCGCATTGCATCAGCCATGATGGATACTTTCTC
GGCAGGAGCAAGGTGAGATGACAGGAGATCCTGCCCCGGCACTTCGCCC
AATAGCAGCCAGTCCCTTCCCGCTTCAGTGACAACGTCGAGCACAGCTG
CGCAAGGAACGCCCGTCGTGGCCAGCCACGATAGCCGCGCTGCCTCGTC
TTGCAGTTCATTCAGGGCACCGGACAGGTCGGTCTTGACAAAAAGAACC
GGGCGCCCCTGCGCTGACAGCCGGAACACGGCGGCATCAGAGCAGCCGA
TTGTCTGTTGTGCCCAGTCATAGCCGAATAGCCTCTCCACCCAAGCGGC
CGGAGAACCTGCGTGCAATCCATCTTGTTCAATCATGCGAAACGATCCT
CATCCTGTCTCTTGATCAGAGCTTGATCCCCTGCGCCATCAGATCCTTG
GCGGCGAGAAAGCCATCCAGTTTACTTTGCAGGGCTTCCCAACCTTACC
AGAGGGCGCCCCAGCTGGCAATTCCGGTTCGCTTGCTGTCCATAAAACC
GCCCAGTCTAGCTATCGCCATGTAAGCCCACTGCAAGCTACCTGCTTTC
TCTTTGCGCTTGCGTTTTCCCTTGTCCAGATAGCCCAGTAGCTGACATT
CATCCGGGGTCAGCACCGTTTCTGCGGACTGGCTTTCTACGTGCTCGAG
GGGGGCCAAACGGTCTCCAGCTTGGCTGTTTTGGCGGATGAGAGAAGAT
TTTCAGCCTGATACAGATTAAATCAGAACGCAGAAGCGGTCTGATAAAA
CAGAATTTGCCTGGCGGCAGTAGCGCGGTGGTCCCACCTGACCCCATGC
CGAACTCAGAAGTGAAACGCCGTAGCGCCGATGGTAGTGTGGGGTCTCC
CCATGCGAGAGTAGGGAACTGCCAGGCATCAAATAAAACGAAAGGCTCA
GTCGAAAGACTGGGCCTTTCGTTTTATCTGTTGTTTGTCGGTGAACGCT
CTCCTGAGTAGGACAAATCCGCCGGGAGCGGATTTGAACGTTGCGAAGC
AACGGCCCGGAGGGTGGCGGGCAGGACGCCCGCCATAAACTGCCAGGCA
TCAAATTAAGCAGAAGGCCATCCTGACGGATGGCCTTTTTGCGTTTCTA
CAAACTCTTTTGTTTATTTTTCTAAATACATTCAAATATGTATCCGCTC
ATGACCAAAATCCCTTAACGTGAGTTTTCGTTCCACTGAGCGTCAGACC
CCGTAGAAAAGATCAAAGGATCTTCTTGAGATCCTTTTTTTCTGCGCGT
AATCTGCTGCTTGCAAACAAAAAAACCACCGCTACCAGCGGTGGTTTGT
TTGCCGGATCAAGAGCTACCAACTCTTTTTCCGAAGGTAACTGGCTTCA
GCAGAGCGCAGATACCAAATACTGTCCTTCTAGTGTAGCCGTAGTTAGG
CCACCACTTCAAGAACTCTGTAGCACCGCCTACATACCTCGCTCTGCTA
ATCCTGTTACCAGTGGCTGCTGCCAGTGGCGATAAGTCGTGTCTTACCG
GGTTGGACTCAAGACGATAGTTACCGGATAAGGCGCAGCGGTCGGGCTG
AACGGGGGGTTCGTGCACACAGCCCAGCTTGGAGCGAACGACCTACACC
GAACTGAGATACCTACAGCGTGAGCTATGAGAAAGCGCCACGCTTCCCG
AAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGT
CCTGTCGGGTTTCGCCACCTCTGACTTGAGCGTCGATTTTTGTGATGCT
CGTCAGGGGGGCGGAGCCTATGGAAAAACGCCAGCAACGCGGCCTTTTT
ACGGTTCCTGGCCTTTTGCTGGCCTTTTGCTCACATGTTCTTTCCTGCG
TTATCCCCTGATTCTGTGGATAACCGTATTACCGCCTTTGAGTGAGCTG
ATACCGCTCGCCGCAGCCGAACGACCGAGCGCAGCGAGTCAGTGAGCGA
GGAAGCGGAAGAGCGCCTGATGCGGTATTTTCTCCTTACGCATCTGTGC
GGTATTTCACACCGCATATGGTGCACTCTCAGTACAATCTGCTCTGATG
CCGCATAGTTAAGCCAGTATACACTCCGCTATCGCTACGTGACTGGGTC
ATGGCTGCGCCCCGACACCCGCCAACACCCGCTGACGCGCCCTGACGGG
CTTGTCTGCTCCCGGCATCCGCTTACAGACAAGCTGTGACCGTCTCCGG
GAGCTGCATGTGTCAGAGGTTTTCACCGTCATCACCGAAACGCGCGAGG
CAGCAGATCAATTCGCGCGCGAAGGCGAAGCGGCATGCATAATGTGCCT
GTCAAATGGACGAAGCAGGGATTCTGCAAACCCTATGCTACTCCGTCAA
GCCGTCAATTGTCTGATTCGTTACCAATTATGACAACTTGACGGCTACA
TCATTCACTTTTTCTTCACAACCGGCACGGAACTCGCTCGGGCTGGCCC
CGGTGCATTTTTTAAATACCCGCGAGAAATAGAGTTGATCGTCAAAACC
AACATTGCGACCGACGGTGGCGATAGGCATCCGGGTGGTGCTCAAAAGC
AGCTTCGCCTGGCTGATACGTTGGTCCTCGCGCCAGCTTAAGACGCTAA
TCCCTAACTGCTGGCGGAAAAGATGTGACAGACGCGACGGCGACAAGCA
AACATGCTGTGCGACGCTGGCGAT

### EXAMPLE 4—SGRNA LIBRARY DESIGN FOR MULTIPLE INTRON (FRAME/PHASE) TAGGING

For tagging two individual genes per cell after two consecutive tagging rounds, wherein a library of genes are to be tagged on the level of the pool of cells, the inventors have designed two libraries targeting the intron frame (also called phase) 0 and frame 1. An intron has the frame 0 (or phase 0), when the exon preceding the intron ends with the third base of a codon and the next exon starts with the first base of the next codon in that gene. In frame 1 (or phase 1) introns, the intron splits a codon between the first and the second base. For generating libraries containing only sgRNAs targeting a certain frame, the inventors used the Ensembl genome browser to obtain transcript information, genomic coordinates of introns and intron frames (FIG. 15a). Intron-targeting sgRNAs were then generated using GuideScan (see e.g., Perez (2017) Nat Biotech 35, 347-349) which uses genomic coordinates as an input. The generated sgRNAs were then ranked based on on- and off-target scores and only sgRNAs for certain frames were selected. Most introns in the human genome are in frame 0 and frame 1 introns are the second most abundant, which is why designed libraries targeted those two intron frames in that example (FIG. 15b). The inventors aimed for a library size that is comparable to genome-wide CRISPR KO libraries and therefore selected the top ranked sgRNA for genes with more than three targeted introns. Two sgRNAs per intron for genes with two or three targeted introns and three sgRNAs per intron for those genes that have only one intron in the selected frame, resulting in a library of 91,058 sgRNAs targeting 74,284 introns of 14,158 genes, including 1000 nontargeting sgRNAs. Using the same strategy, the inventors designed a library of 72,926 sgRNAs targeting 52,938 introns in the frame 1 of 14,011 genes (FIG. 15c).

### EXAMPLE 5—MINICIRCLE CONSTRUCTS FOR MULTIPLE INTRON TAGGING

The inventors have ameliorated and cloned minicircle constructs on the basis of minicircle constructs disclosed in WO 2021/099273. These novel minicircle constructs contain acceptor/donor flanked fluorescent proteins of different colours (i.e. the tag sequences of the present invention) in the different reading frames. The (E)GFP minicircle (which represents a minicircle containing an illustrative (E)GFP tag of the embodiments) for targeting frame 0 introns for example, does not contain any frame correcting bases and the coding sequence after the splice acceptor sequence starts with the first base of a codon and ends with the third base of a codon before the splice donor sequence (FIG. 16a, 16b). The mScarlet minicircle (which again represents a minicircle containing an illustrative mScarlet tag of the embodiments) for targeting frame 1 introns for example, contains two extra bases at the start of the coding sequence after the splice acceptor and one extra base at the end of the coding sequence before the splice donor (FIG. 16c, 16d). Guanines were added as extra bases into the constructs to avoid the introduction of any premature stop codons after genomic integration of the tag sequence (e.g, the first base of the last codon of the preceding exon (which is either A, U, G or C) forms a codon with the two extra Gs after integration, the codon is either one of AGG, UGG, GGG or CGG, all of which code for glycine).

Annotation and sequence of the mScarlet frame 1 minicircle production plasmid:

| | |
|---|---|
| 1-738 | 32 consecutive ISce1 site |
| 782-816 | attB site |
| 852-871 | generic sgRNA target site |
| 872-874 | PAM site |
| 875-916 | splice acceptor |
| 917-918 | two extra bases GG to match frame 1 introns |
| 919-978 | 20-amino-acid-linker |
| 979-1671 | mScarlet |
| 1672-1737 | 21-amino-acid-linker |
| 1738-1738 | one extra base G to match frame 1 introns |
| 1739-1758 | splice donor |
| 1794-1832 | attP site |
| 1919-2713 | Kanamycin resistance |
| 3016-5053 | EcoE1 origin |

(SEQ ID NO: 68)
ACATTACCCTGTTATCCCTAGATACATTACCCTGTTATCCCAGATGACA
TACCCTGTTATCCCTAGATGACATTACCCTGTTATCCCAGATGACATTA
CCCTGTTATCCCTAGATACATTACCCTGTTATCCCAGATGACATACCCT
GTTATCCCTAGATGACATTACCCTGTTATCCCAGATGACATTACCCTGT
TATCCCTAGATACATTACCCTGTTATCCCAGATGACATACCCTGTTATC
CCTAGATGACATTACCCTGTTATCCCAGATGACATTACCCTGTTATCCC
TAGATACATTACCCTGTTATCCCAGATGACATACCCTGTTATCCCTAGA
TGACATTACCCTGTTATCCCAGATGACATTACCCTGTTATCCCTAGATA
CATTACCCTGTTATCCCAGATGACATACCCTGTTATCCCTAGATGACAT
TACCCTGTTATCCCAGATGACATTACCCTGTTATCCCTAGATACATTAC
CCTGTTATCCCAGATGACATACCCTGTTATCCCTAGATGACATTACCCT
GTTATCCCAGATGACATTACCCTGTTATCCCTAGATACATTACCCTGTT
ATCCCAGATGACATACCCTGTTATCCCTAGATGACATTACCCTGTTATC
CCAGATGACATTACCCTGTTATCCCTAGATACATTACCCTGTTATCCCA
GATGACATACCCTGTTATCCCTAGATGACATTACCCTGTTATCCCAGAT
AAACTCAATGATGATGATGATGATGGTCGAGACTCAGCGGCCGCGGTGC
CAGGGCGTGCCCTTGGGCTCCCCGGGCGCGACTAGTGAATTCAGATCTG
ATCCTGCGTTATCCCCTGGAGATCGAGTGCCGCATCACCGGCTATTGGT
CTTACTGACATCCACTTTGCCTTTCTCTCCACAGGGGGTGGCGGTGGCT
CGGGCGGTGGTGGGTCCGGTGGCGGCGGATCTGGCGGTGGTGGATCCGT
GAGCAAGGGCGAGGCAGTGATCAAGGAGTTCATGCGGTTCAAGGTGCAC
ATGGAGGGCTCCATGAACGGCCACGAGTTCGAGATCGAGGGCGAGGGCG
AGGGCCGCCCCTACGAGGGCACCCAGACCGCCAAGCTGAAGGTGACCAA
GGGTGGCCCCCTGCCCTTCTCCTGGGACATCCTGTCCCCTCAGTTCATG
TACGGCTCCAGGGCCTTCATCAAGCACCCCGCCGACATCCCCGACTACT
ATAAGCAGTCCTTCCCCGAGGGCTTCAAGTGGGAGCGCGTGATGAACTT
CGAGGACGGCGGCGCCGTGACCGTGACCCAGGACACCTCCCTGGAGGAC
GGCACCCTGATCTACAAGGTGAAGCTCCGCGGCACCAACTTCCCTCCTG
ACGGCCCCGTAATGCAGAAGAAGACAATGGGCTGGGAAGCGTCCACCGA
GCGGTTGTACCCCGAGGACGGCGTGCTGAAGGGCGACATTAAGATGGCC
CTGCGCCTGAAGGACGGCGGCCGCTACCTGGCGGACTTCAAGACCACCT
ACAAGGCCAAGAAGCCCGTGCAGATGCCCGGCGCCTACAACGTCGACCG
CAAGTTGGACATCACCTCCCACAACGAGGACTACACCGTGGTGGAACAG
TACGAACGCTCCGAGGGCCGCCACTCCACCGGCGGCATGGACGAGCTGT
ACAAGGGCGGCGGTGGATCCGGTGGCGGCGGATCTGGCGGTGGAGGTTC
GGGTGGCGGTGGGTCGGGCGGAGGTAAGTATCAATTAGAAGACGAATTC
CATCTCTAGAGTCGACCCATGGGGGCCCGCCCCAACTGGGGTAACCTTT
GAGTTCTCTCAGTTGGGGGTAATCAGCATCATGATGTGGTACCACATCA
TGATGCTGATTATAAGAATGCGGCCGCCACACTCTAGTGGATCTCGAGT
TAATAATTCAGAAGAACTCGTCAAGAAGGCGATAGAAGGCGATGCGCTG
CGAATCGGGAGCGGCGATACCGTAAAGCACGAGGAAGCGGTCAGCCCAT
TCGCCGCCAAGCTCTTCAGCAATATCACGGGTAGCCAACGCTATGTCCT
GATAGCGGTCCGCCACACCCAGCCGGCCACAGTCGATGAATCCAGAAAA
GCGGCCATTTTCCACCATGATATTCGGCAAGCAGGCATCGCCATGGGTC
ACGACGAGATCCTCGCCGTCGGGCATGCTCGCCTTGAGCCTGGCGAACA
GTTCGGCTGGCGCGAGCCCCTGATGCTCTTCGTCCAGATCATCCTGATC
GACAAGACCGGCTTCCATCCGAGTACGTGCTCGCTCGATGCGATGTTTC
GCTTGGTGGTCGAATGGGCAGGTAGCCGGATCAAGCGTATGCAGCCGCC
GCATTGCATCAGCCATGATGGATACTTTCTCGGCAGGAGCAAGGTGAGA
TGACAGGAGATCCTGCCCCGGCACTTCGCCCAATAGCAGCCAGTCCCTT
CCCGCTTCAGTGACAACGTCGAGCACAGCTGCGCAAGGAACGCCCGTCG
TGGCCAGCCACGATAGCCGCGCTGCCTCGTCTTGCAGTTCATTCAGGGC
ACCGGACAGGTCGGTCTTGACAAAAAGAACCGGGCGCCCCTGCGCTGAC
AGCCGGAACACGGCGGCATCAGAGCAGCCGATTGTCTGTTGTGCCCAGT
CATAGCCGAATAGCCTCTCCACCCAAGCGGCCGGAGAACCTGCGTGCAA
TCCATCTTGTTCAATCATGCGAAACGATCCTCATCCTGTCTCTTGATCA
GAGCTTGATCCCCTGCGCCATCAGATCCTTGGCGGCGAGAAAGCCATCC
AGTTTACTTTGCAGGGCTTCCCAACCTTACCAGAGGGCGCCCCAGCTGG
CAATTCCGGTTCGCTTGCTGTCCATAAAACCGCCCAGTCTAGCTATCGC
CATGTAAGCCCACTGCAAGCTACCTGCTTTCTCTTTGCGCTTGCGTTTT
CCCTTGTCCAGATAGCCCAGTAGCTGACATTCATCCGGGGTCAGCACCG
TTTCTGCGGACTGGCTTTCTACGTGCTCGAGGGGGGCCAAACGGTCTCC
AGCTTGGCTGTTTTGGCGGATGAGAGAAGATTTTCAGCCTGATACAGAT
TAAATCAGAACGCAGAAGCGGTCTGATAAAACAGAATTTGCCTGGCGGC
AGTAGCGCGGTGGTCCCACCTGACCCCATGCCGAACTCAGAAGTGAAAC
GCCGTAGCGCCGATGGTAGTGTGGGGTCTCCCCATGCGAGAGTAGGGAA
CTGCCAGGCATCAAATAAAACGAAAGGCTCAGTCGAAAGACTGGGCCTT
TCGTTTTATCTGTTGTTTGTCGGTGAACGCTCTCCTGAGTAGGACAAAT
CCGCCGGGAGCGGATTTGAACGTTGCGAAGCAACGGCCCGGAGGGTGGC
GGGCAGGACGCCCGCCATAAACTGCCAGGCATCAAATTAAGCAGAAGGC
CATCCTGACGGATGGCCTTTTTGCGTTTCTACAAACTCTTTTGTTTATT
TTTCTAAATACATTCAAATATGTATCCGCTCATGACCAAAATCCCTTAA
CGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTA
CCAACTCTTTTTCCGAAGGTAACTGGCTTCAGCAGAGCGCAGATACCAA
ATACTGTCCTTCTAGTGTAGCCGTAGTTAGGCCACCACTTCAAGAACTC
TGTAGCACCGCCTACATACCTCGCTCTGCTAATCCTGTTACCAGTGGCT
GCTGCCAGTGGCGATAAGTCGTGTCTTACCGGGTTGGACTCAAGACGAT
AGTTACCGGATAAGGCGCAGCGGTCGGGCTGAACGGGGGGTTCGTGCAC
ACAGCCCAGCTTGGAGCGAACGACCTACACCGAACTGAGATACCTACAG
CGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCT
TCCAGGGGGAAACGCCTGGTATCTTTATAGTCCTGTCGGGTTTCGCCAC
CTCTGACTTGAGCGTCGATTTTTGTGATGCTCGTCAGGGGGGCGGAGCC
TATGGAAAAACGCCAGCAACGCGGCCTTTTTACGGTTCCTGGCCTTTTG
CTGGCCTTTTGCTCACATGTTCTTTCCTGCGTTATCCCCTGATTCTGTG
GATAACCGTATTACCGCCTTTGAGTGAGCTGATACCGCTCGCCGCAGCC
GAACGACCGAGCGCAGCGAGTCAGTGAGCGAGGAAGCGGAAGAGCGCCT
GATGCGGTATTTTCTCCTTACGCATCTGTGCGGTATTTCACACCGCATA
TGGTGCACTCTCAGTACAATCTGCTCTGATGCCGCATAGTTAAGCCAGT
ATACACTCCGCTATCGCTACGTGACTGGGTCATGGCTGCGCCCCGACAC
CCGCCAACACCCGCTGACGCGCCCTGACGGGCTTGTCTGCTCCCGGCAT
CCGCTTACAGACAAGCTGTGACCGTCTCCGGGAGCTGCATGTGTCAGAG
GTTTTCACCGTCATCACCGAAACGCGCGAGGCAGCAGATCAATTCGCGC
GCGAAGGCGAAGCGGCATGCATAATGTGCCTGTCAAATGGACGAAGCAG
GGATTCTGCAAACCCTATGCTACTCCGTCAAGCCGTCAATTGTCTGATT
CGTTACCAATTATGACAACTTGACGGCTACATCATTCACTTTTTCTTCA
CAACCGGCACGGAACTCGCTCGGGCTGGCCCCGGTGCATTTTTTAAATA
CCCGCGAGAAATAGAGTTGATCGTCAAAACCAACATTGCGACCGACGGT
GGCGATAGGCATCCGGGTGGTGCTCAAAAGCAGCTTCGCCTGGCTGATA
CGTTGGTCCTCGCGCCAGCTTAAGACGCTAATCCCTAACTGCTGGCGGA
AAAGATGTGACAGACGCGACGGCGACAAGCAAACATGCTGTGCGACGCT
GGCGAT

### EXAMPLE 6—GENERATION OF A POOL OF CELLS COMPRISING CELLS WITH TWO TAGGED INTRONS (I.E., TWO ROUNDS OF INTRON TAGGING) WHICH ARE FURTHER DISTINGUISHABLE BY THE USE OF STRUCTURAL MARKERS

The inventors have performed two rounds of intron tagging in HAP1 cells to generate a pool of cells wherein in every cell two individual genes were tagged (FIG. 17) (and wherein gene identity between cells varied thus covering a library of genes). In a non-limiting illustrative example, they first transduced cells with the intron-targeting sgRNA library targeting frame 0 at a low MOI (0.05), and selected cells via an antibiotic selection marker (puromycin in this case) to eliminate cells that were not transduced and which did not genomically integrate an intron targeting sgRNA together with an antibiotic resistance gene (puromycin in this case). Cells were subsequently transfected with the first frame 0 minicircle encoding the first fluorescent tag ((E)GFP) together with a plasmid encoding Cas9 and a generic, minicircle linearizing sgRNA which led to the integration of the (E)GFP tag into target introns. (E)GFP positive cells were then sorted to obtain a pool of cells, wherein in every cell one individual gene was tagged with (E)GFP. This pool of cells was then transduced with a second sgRNA library targeting frame 1 introns, again followed by antibiotics selection (blasticidin in this case) to eliminate cells that were not transduced and which did not genomically integrate an intron targeting sgRNA together with the second antibiotics resistance gene (blasticidin in this case). Being able to use two different antibiotic selection markers is another advantage of using two independent sgRNA libraries compared to using the same library twice. Furthermore, if the same sgRNA library would be used twice, antibiotic selection in the second round would not be possible (all cells would already be resistant to the antibiotic) and cells that were not transduced and did not genomically integrate a second sgRNA sequence would remain in the pool of cells, thereby lowering the overall editing efficiency. After the second antibiotics selection step, cells transduced with the frame 1 sgRNA library were then transfected with a second minicircle construct encoding the second fluorescent tag (mScarlet) for targeting the intron frame 1 and a plasmid expressing Cas9 and a generic sgRNA targeting and linearizing the minicircle construct. Finally, cells that were double positive for both fluorescent tags (i.e., (E)GFP and mScarlet double positive) were sorted to obtain a pool of cells, wherein in every cell, two individual genes were tagged with either of both fluorescent tags. The intron tagging experiments were carried out at a relatively large scale (transfection and sorting of hundreds of millions of cells, see FIG. 17 for the exact number of cells that were transduced/transfected and sorted in each step), to sort a sufficiently high number of (E)GFP and mScarlet double positive cells, despite the overall tagging efficiency in HAP1 cells being relatively low (see paragraph below), mainly because HAP1 represents a difficult to transfect cell line.

As expected, the tagging efficiency in cells that were transduced using the frame 1 sgRNA library and transfected with the matching minicircle construct was lower compared to cells that were transduced with a single intron-targeting sgRNA targeting MTHFD2 (positive control, 0.0039% vs 0.152%, FIG. 18a). This difference is to be expected because the majority of sgRNAs in the sgRNA library do not lead to detectable levels of expressed gene tagged proteins for various reasons, such as low expression of the target gene or tagging of the protein at a particular site leading to misfolding and proteasomal degradation of the protein. However, the tagging efficiency was higher in cells transduced with the frame 1 sgRNA library in the second round of tagging compared to cells that were not transfected with a second intron-targeting sgRNA (0.0039% vs. 0.0026%). The difference in the tagging efficiency between these two experimental conditions indicates that there was specific, intron-targeting sgRNA dependent integration in transduced cells compared to random integration or integration at the target site of the first round of tagging in the cells without a second sgRNA. Importantly, the inventors did not observe this difference when performing the second round of tagging with the same sgRNA library that was used for the first round of tagging, indicating specific, intron-targeting sgRNA dependent integration (FIG. 18b). Instead, a diagonal line in FACS plots indicates cells exhibiting the same fluorescence signal intensity in the channels for the first and second tag sequence (i.e., the (E)GFP and the mScarlet tags). This observation indicates that within those cells, the same gene was tagged with both tags (i.e, (E)GFP and mScarlet), however on different alleles. This cannot happen when using sgRNA libraries targeting different frames and using matching minicircle constructs, because integration of a frame 1 minicircle at the target site of a frame 0 sgRNA would lead to a frameshift instead of the expression of a tagged gene.

To confirm that the inventors had obtained a highly diverse pool of cells, they isolated genomic DNA from the pool of the cells that were intron tagged twice, PCR amplified the sgRNA containing/encoding genomic regions and performed NGS-based amplicon sequencing. The sequencing reads were mapped to the two sgRNA libraries as expected and it was confirmed that a high diversity of intron-targeting sgRNAs was present in the pool of cells (927 different sgRNAs that map to the frame 0 sgRNA library and 987 sgRNAs that map to the frame 1 sgRNA library), while non-targeting sgRNAs were depleted from the pool (6 non-targeting sgRNAs were detected in the pool, none of them among the top ranked, most abundant sgRNAs) (FIG. 19a, 19b, 19c).

In the next step, the inventors transduced the highly diverse pool of cells with a lentiviral construct for stably integrating and overexpressing an additional marker protein (i.e., blue fluorescent protein, specifically mTagBFP2) which localized to one of several different cellular organelles (FIG. 17). This was achieved by fusing the coding sequence of the mTagBFP2 marker to either a nuclear localization sequence (NLS), a nuclear export sequence (NES), a mitochondrial localization sequence or fused to H2B (fusion of mTagBFP2 to any subcellular localization sequence or protein that has a specific subcellular localization pattern can be used here). The purpose of this additional marker protein was to better phenotypically discriminate a large number of clones that would otherwise look very similar if only two genes would be tagged per cell after the two rounds of intron tagging. Furthermore, the pool of cells was transduced with lentiviral constructs for stably integrating and overexpressing structural markers that aid in cell segmentation during image analysis of microscopic images. To illustrate, the inventors in this example used the far-red fluorescent protein marker miRFP670 fused to a nuclear localization sequence and the fluorescent protein marker mAmetrine fused to a prenylation sequence for localization to the cell membrane. In addition to aiding in cell segmentation, the differences in the expression levels and resulting fluorescence intensity of these structural markers could be used to better discriminate clones in the pool of cells.

### EXAMPLE 7—IMPLEMENTATION OF AUTOMATED CLONE RECOGNITION

To eventually use computer vison to identify the different clones within the highly diverse pool of cells, it is necessary to first train a computational model. Ideally, this is done using images obtained from single clonal cell lines in which the identity of the two tagged proteins in a given clone is known. In order to obtain these images for training a model, the inventors isolated, imaged and (sgRNA) genotyped more than 2000 individual clonal cell lines from the pool of intron-tagged cells (FIG. 20a, 20b, 20c). This was done at high-throughput using equipment for plate handling and processing. To determine the two tagged genes in each isolated clonal cell line the inventors performed NGS based barcoded (sgRNA) amplicon sequencing for each well (for each single clone).

For proof of concept, the inventors then used the images of the isolated clonal cell lines to train computational models to recognize clones in the pool of cells (FIG. 21) based on their phenotype and gRNA identity. First, cell segmentation was performed on the images of clonal cell lines using the Cellpose algorithm (Stringer (2021) Nat Methods 18, 100-106) to obtain images of single cells. Next, features such as intensity values, granularity, texture, etc. were extracted for each cell in the different channels as well as for the certain organelles such as nucleus and cytoplasm using CellProfiler (McQuin (2018) PLOS Biol 16, e2005970). In total, 1576 features were extracted for each cell and used to train a random forest model using the scikit-learn library (Pedregosa (2011) J Machine Learning Res 12, 2825-2830) in the Python programming language with default parameters. The model was then used to predict the identity of the clone in a separate set of test images of single cells that were not used for model training. Single cells could be assigned to the correct identity (i.e., gene/gRNA identity) with 98% accuracy. Importantly, isolation of clonal cell lines to train a model is done only once for each pool of cells within the present invention. The model trained on isolated clones may then be used to identify clones either in the original highly diverse pool of cells or in any other pool of cells that is generated by re-pooling selected clonal cell lines.

1. Method for obtaining an intron-tagged pool of cells representing and/or comprising tagged introns, said method comprising the steps of:
(a) selecting introns to be targeted in the genome of a cell;
(b) identifying guide RNA (gRNA) sequences suitable for inserting a tag sequence in the selected introns in the genome of a cell to be intron tagged;
(c) cloning identified gRNA sequences and tag sequence into transfection and/or transduction vectors;
(d) contacting a population of the cells to be intron-tagged with said transfection and/or transduction vectors of (c);
(e) selecting of intron-tagged cells based on the presence of the tag sequence; and
(f) obtaining a pool of cells representing a plurality of tagged introns
wherein steps (b) to (e) are repeatedly performed using a unique combination of the gRNA sequences and the tag sequence for each round of repetition.

2. The method of claim 1, wherein in step (d) said transfection and/or transduction vectors comprising said gRNA sequences integrate the gRNA sequences into the genome of the transfected and/or transduced cells.

3. The method of claims 1 and 2, wherein a single gRNA sequence is integrated into the genome of the individual transfected and/or transduced cell within the population of the transfected and/or transduced cells.

4. The method of any one of claims 1 to 3, wherein in step (d) said transfection and/or transduction vector comprising said tag sequence integrate the tag sequence into the genome of the transfected and/or transduced cells.

5. The method of any one of claims 1 to 4, wherein said selecting of intron-tagged cells comprises fluorescence-activated cell sorting (FACS).

6. The method of any one of claims 1 to 5, wherein said pool of cells contains at least 2, at least 5, at least 10, at least 20, at least 50, at least 100, at least 200, at least 300, at least 400, at least 500, at least 1000, at least 1500, at least 2000, at least 2500, at least 3000 at least 10000 tagged introns.

7. The method of any one of claims 1 to 6, wherein steps (b) to (e) are performed 2 times, at least 3 times, at least 4 times, at least 5 times or at least 6 times.

8. The method of any one of claims 1 to 7, wherein the tag for each round of repetition is a different fluorescent tag, selected from the group consisting of GFP, EGFP, YFP, RFP, mScarlet, BFP or is selected from a tag suitable for detection by covalent (e.g. Halo tag, Clip tag, Snap tag, Spy tag) or non-covalent (e.g. Strep-tag, HA tag, dTag) binding to a detection reagent enabling detection by microscopy, e.g. by fluorescence or luminescence.

9. The method of any one of claims 1 to 8, wherein before step (f) (a) further fluorescent and/or luminescent marker(s) selected from the group consisting of miRFP670, mAmetrine, (mTag)BFP(2), fluorescently labeled antibodies, DAPI, Hoechst 33258, Hoechst 33342 and Hoechst 34580 is/are used to label the cell and/or cellular substructures within the individual cell comprising the tagged-introns.

10. The method of any one of claims 1 to 9, wherein the gRNA sequences for each round of repetition target a different one of three intron frames or phases and/or a different one of three exonic open reading frames.

11. The method of claim 10, wherein said intron frames and/or exonic open reading frames was/were not used in a previous round of repetition.

12. The method of any one of claims 1 to 11, wherein in step (c) the identified gRNA sequences are cloned into a transduction vector and the tag sequence is cloned into a transfection vector, preferably a transfection vector that allows the production of minicircle DNA.

13. The method of any one of claims 1 to 12, wherein the transduction vector is a sequencing vector, preferably a CROP-Seq vector.

14. The method of any one of claims 1 to 13, wherein the introns to be targeted are comprised in genomic sequences of metabolic enzymes, chromatin proteins, kinases, genes coding for proteins in the ubiquitin/proteasome pathways, transcription factors, ion channels, transporters, receptors or wherein at least one intron per protein coding gene in the genome is targeted.

15. The method of any one of claims 1 to 14, wherein the introns to be targeted are selected based on the reading frame of the upstream exonic sequence, wherein the sequence to be inserted is in-frame with the exonic sequence.

16. The method of any one of claims 1 to 15, wherein the gRNA sequences suitable for inserting a tag sequence in the selected introns in the genome of the cell are identified according to Cas9 cutting efficiency or Cpf1 cutting efficiency or Cas12b cutting efficiency.

17. The method of any one of claims 1 to 16, wherein the gRNA sequences suitable for inserting a tag sequence in the selected introns in the genome of the cell are identified according to their occurrence in the genome of the cell, preferably wherein the occurrence is 1.

18. The method of any one of claims 1 to 17, wherein the gRNA is a single gRNA (sgRNA).

19. The method of any one of claims 1 to 18, wherein the cell is a HAP1 cell, K562 cell, HeLa cell, KBM7 cell, BT474 cell, MG-63 cell, SKNAS cell, A427 cell, A375 cell, A498 cell, RCH-ACV cell, HEK293T cell, A673 cell, SK-N-MC cell, A549 cell, SKMES1 cell, NCIH727 cell, THP1 cell, NB4 cell, MOLM13 cell, KASUMI-1 cell, HEL cell, NB-4 cell, HL-60 cell, RS4-11 cell, MOLT7 cell, aTC1 cell, bTC3 cell, Min6 cell or another cell line, preferentially an adherent, non-migratory cell line.

20. The method of any one of claims 1 to 19, wherein the cell is a U-2 OS cell.

21. The method of any one of claims 1 to 20, wherein the intron-tagged pool of cells comprises intron and/or exon tagged cells.

22. An intron-tagged pool of cells obtainable by the method of any one of claims 1 to 21.

23. The intron-tagged pool of cells of claim 22, wherein the individual cell in said intron-tagged pool of cells is characterized by at least two different tags in at least two different intron frames or phases of at least two different genes.

24. A pool of cells comprising a plurality of intron-tagged cells, wherein an individual cell, preferably each individual cell, in said pool of cells is characterized by at least two different tags in at least two different introns of at least two different genes.

25. The pool of cells of claim 24, wherein an individual cell, preferably each individual cell, in said pool of cells comprises at least two different tags in at least two different intron frames of at least two different genes.

26. The pool of cells of claim 25, wherein an individual cell, preferably each individual cell, in said pool of cells comprises two different tags in two different intron frames of two different genes, or three different tags in three different intron frames of three different genes.

27. The pool of cells of claim 25, wherein an individual cell, preferably each individual cell, in said pool of cells comprises (i) two different tags in two introns of two different genes, wherein the two introns have different intron frames, or (ii) three different tags in three introns of three different genes, wherein each of the three introns has a different intron frame; and wherein the plurality of intron-tagged cells in the pool of cells comprises at least 10 or at least 100 tagged introns.

28. The pool of cells of any one of claims 24 to 27, wherein the tags are selected from fluorescent tags such as GFP, or tags suitable for detection by covalent or non-covalent binding to a detection reagent enabling detection by microscopy, e.g. by fluorescence or luminescence, such as a Halo tag or a Strep-tag.

29. The pool of cells of any one of claims 24 to 28, wherein said pool of cells and/or said plurality of intron-tagged cells comprises at least 10, 100, 1000, 10000 or 20000 tagged introns.

30. The pool of cells of any one of claims 24 to 29, wherein a gene, preferably each gene, comprising a tagged intron can be translated into a corresponding fusion protein.

31. The pool of cells of any one of claims 24 to 30, wherein an individual cell, preferably each individual cell, in said pool of cells further comprises at least one tag in at least one exonic open reading frame, preferably in two or three different exonic open reading frames, preferably in two of three different genes.

32. The pool of cells of any one of claims 24 to 31, wherein at least one cellular substructure is labelled in at least one or all of the intron tagged cells.

33. The pool of cells of any one of claims 24 to 32, wherein at least one or all of the intron tagged cells and/or at least one cellular substructure thereof is/are labelled with at least one further fluorescent and/or luminescent marker selected from the group consisting of miRFP670, mAmetrine, (mTag)BFP(2), fluorescently labeled antibodies, DAPI, Hoechst 33258, Hoechst 33342 and Hoechst 34580.

34. The pool of cells of any one of claims 24 to 33, wherein the pool of cells is obtainable by or obtained by the method of any one of claims 1 to 21.

35. A method for automated recognition of the identity of tagged intron(s) comprised in the genome of an intron-tagged cell within a pool of cells of any one of claims 22 to 34, said method comprising the steps of:
(a) identifying said intron-tagged cells comprising genomically tagged introns by obtaining (a) single-cell microscopy image(s) of the cell, said image(s) capturing fluorescent and/or luminescent signal(s) emitted from
(i) the expressed tag sequence(s) of the genomically tagged introns and/or
(ii) labeled cellular substructure(s) and/or organelle(s);
(b) sequencing the gRNA sequences, or parts thereof, integrated into the genome of the intron-tagged cells and as identified in step (a);
(c) training (a) model(s) of a computer vision algorithm based on features from image(s) and gRNA sequencing data obtained in (a) and (b); and
(d) automatically recognizing the identity of the tagged introns comprised in the intron-tagged cells within the pool of cells.

36. The method of claim 35, wherein the substructure(s) and/or organelle(s) are selected from the group consisting of cytoskeleton, cell membrane, cell wall, chloroplast, endoplasmic reticulum, golgi apparatus, mitochondrion, and nucleus.

37. The method of claim 35 or 36, wherein said substructure(s) and/or organelle(s) are labeled with fluorescent and/or luminescent marker(s) selected from the group consisting of miRFP670, mAmetrine, (mTag)BFP(2), fluorescently labeled antibodies, DAPI, Hoechst 33258, Hoechst 33342 and Hoechst 34580.

38. The method of any one of claims 35 to 37, wherein the computer vision algorithm is based on machine learning.

39. The method of any one of claims 35 to 38, wherein the model(s) is/are based on random forests, support vector machines, variational autoencoders, recurrent neural networks (RNN), restricted Boltzmann machines, and/or convolutional neural networks (CNN).

40. The method of any one of claims 35 to 39, wherein the features comprise
(a) the texture and granularity of cells, the presence, absence, intensity, distribution and/or localization of fluorescent and/or luminescent signals; and
(b) the identity of the tagged introns per cell.

41. The method of any one of claims 35 to 40, wherein the identity of the tagged introns of the intron-tagged cells is recognized with at least 95% accuracy, preferably with 98% accuracy.

42. Use of the pool of cells of any one of claims 22 to 34 for drug screening.

43. Use of the pool of cells of any one of claims 22 to 34 for assessing the effects of environmental factors such as toxic compounds on cells.

44. A method for assessing the effect of a perturbation on the proteome and/or gene expression levels of an intron-tagged cell within a pool of cells of any one of claims 22 to 34, said method comprising the steps of:
(a) exposing the intron-tagged cells within the pool of cells to a perturbation;
(b) obtaining single-cell resolved time-course microscopy images of the intron-tagged cells within the pool of cells, said images capturing fluorescent and/or luminescent signal(s) emitted from
(i) the expressed tag sequence(s) of the genomically tagged introns and/or
(ii) labeled cellular substructure(s) and/or organelle(s); and
(c) assessing the effect of the perturbation based on single-cell analysis of the expressed tag sequence(s) of the genomically tagged introns and the labeled cellular substructure(s) and/or organelle(s) prior and after said perturbation.

45. A method for assessing the effect of a perturbation on the proteome and/or gene expression levels of an intron-tagged cell within a pool of cells of any one of claims 22 to 34, said method comprising the steps of:
(a) selecting introns to be targeted in the genome of a cell;
(b) identifying guide RNA (gRNA) sequences suitable for inserting a tag sequence in the selected introns in the genome of a cell to be intron-tagged;
(c) cloning identified gRNA sequences and tag sequence into transfection and/or transduction vectors;
(d) contacting a population of the cells to be intron-tagged with said transfection and/or transduction vectors of (c);
(e) selecting of intron-tagged cells based on the presence of the tag sequence;
(f) repeatedly performing steps (b) to (e) using a unique combination of the gRNA sequences and the tag sequence for each round of repetition, wherein said steps are performed at least 2 times, at least 3 times, at least 4 times, at least 5 times, or at least 6 times, and wherein (a) further fluorescent and/or luminescent marker(s) is/are used after the final round of repetition to label cellular substructure(s) and/or organelle(s) of the cell comprising the tagged introns;
(g) identifying cells comprising genomically tagged introns by obtaining (a) single-cell microscopy image(s) of the intron-tagged cells, said image(s) capturing fluorescent and/or luminescent signal(s) emitted from
(i) the expressed tag sequence(s) of the genomically tagged introns and/or
(ii) labeled cellular substructure(s) and/or organelle(s);
(h) automatically recognizing the identity of the tagged introns comprised in the intron-tagged cells within the intron-tagged pool of cells;
(i) exposing the intron-tagged cells within the pool of cells to a perturbation;
(j) obtaining single-cell resolved time-course microscopy images of the intron-tagged cells within the pool of cells, said images capturing fluorescent and/or luminescent signal(s) emitted from
(i) the expressed tag sequence(s) of the genomically tagged introns and/or
(ii) labeled cellular substructure(s) and/or organelle(s); and
(k) assessing the effect of the perturbation based on single-cell analysis of the expressed tag sequence(s) of the genomically tagged introns and the labeled cellular substructure(s) and/or organelle(s) prior and after said perturbation.

46. The method of claim 45, wherein in step (d) said transfection and/or transduction vectors comprising said gRNA sequences integrate the gRNA sequences into the genome of the transfected and/or transduced cells.

47. The method of claim 45 or 46, wherein a single gRNA sequence is integrated into the genome of the individual transfected and/or transduced cell within the population of the transfected and/or transduced cells.

48. The method of any one of claims 45 to 47, wherein in step (d) said transfection and/or transduction vector comprising said tag sequence integrate the tag sequence into the genome of the transfected and/or transduced cells.

49. The method of any one of claims 45 to 48, wherein said selecting of intron-tagged cells in step (e) comprises fluorescence-activated cell sorting (FACS).

50. The method of any one of claims 45 to 49, wherein the tag for each round of repetition is a different fluorescent tag selected from the group consisting of GFP, EGFP, YFP, RFP, mScarlet, BFP or is selected from a tag suitable for detection by covalent or non-covalent binding to a detection reagent enabling detection by microscopy, e.g., by fluorescence or luminescence, such as a Halo tag, Clip tag, Snap tag, Strep-tag, HA tag, or dTag.

51. The method of any one of claims 44 to 50, wherein the cellular substructure(s) and/or organelle(s) is/are selected from the group consisting of cytoskeleton, cell membrane, cell wall, chloroplast, endoplasmic reticulum, golgi apparatus, mitochondrion, and nucleus.

52. The method of any one of claims 45 to 51, wherein the gRNA sequences for each round of repetition target one of three intron frames or phases and/or one of three exonic open reading frames

53. The method of any one of claims 45 to 52, wherein in step (c) the identified gRNA sequences are cloned into a transduction vector and the tag sequence is cloned into a donor plasmid, preferably a donor plasmid that allows the production of minicircle DNA.

54. The method of any one of claims 44 to 53, wherein the perturbation is selected from radiation, an inorganic chemical compound, an organic chemical compound, a biological compound, temperature, nutrient depletion, and ion concentration(s).

55. The method of any one of claims 44 to 54, wherein the method is used for testing drugs.

56. The method of any one of claims 44 to 54, wherein the method is used for testing drugs for use in the treatment of cancer and/or neurological diseases.

57. The method of any one of claims 45 to 56, wherein single-cell analysis of the expressed tag sequence(s) of the genomically tagged introns is based on the alteration of presence, absence, amount, distribution and/or localization of said expressed tag sequence(s) of the intron-tagged cells within the pool of cells.

58. The pool of cells of any one of claims 22 to 34 for use in a method for automated recognition of the identity of tagged intron(s) comprised in the genome of an intron-tagged cell within a pool of cells according to any one of claims 35 to 41.

59. The pool of cells of any one of claims 22 to 34 for use in a method for assessing the effect of a perturbation on the proteome and/or gene expression levels of an intron-tagged cell within a pool of cells according to any one of claims 44 to 57.
